(19) United States
(12) Patent Application Publication
(10) Pub. No.: US 2024/0094224 A1
(43) Pub. Date: Mar. 21, 2024

(54) ANTI-MICROBIAL PEPTIDE (AMP) SIGNATURES FOR DIAGNOSTICS, STRATIFICATION, AND TREATMENT OF MICROBIOME-ASSOCIATED DISEASE

(71) Applicant: Yeda Research and Development Co. Ltd., Rehovot (IL)

(72) Inventors: Eran ELINAV, Mazkeret Batya (IL); Alon SAVIDOR, Rehovot (IL)

(73) Assignee: Yeda Research and Development Co. Ltd., Rehovot (IL)

(21) Appl. No.: 18/522,466

(22) Filed: Nov. 29, 2023

Related U.S. Application Data
(63) Continuation of application No. PCT/IL2022/050574, filed on May 30, 2022.

(30) Foreign Application Priority Data
May 30, 2021 (IL) 283563

(51) Int. Cl. G01N 33/68

(52) U.S. Cl. CPC G01N 33/6893; G01N 2333/4721; G01N 2800/065; G01N 2800/26; G01N 2800/60

(57) ABSTRACT

Proxies for physiological states of tissue are provided. Accordingly, there are provided methods of determining host AMP landscape, establishing a profile indicative of tissue status, and identifying proxies for tissue status in AMP profiles of samples of secretions, exudates and/or excretions of the tissue. Also provided are methods for determination of disease severity and chronology.

### RELATED APPLICATIONS

This application is a Continuation of PCT Patent Application No. PCT/IL2022/050574 having International filing date of May 30, 2022, which claims the benefit of priority of Israel Patent Application No. 283563 filed on May 30, 2021. The contents of the above applications are all incorporated by reference as if fully set forth herein in their entirety.

### SEQUENCE LISTING STATEMENT

The XML file, entitled 98098SequenceListing.xml, created on Nov. 15, 2023, comprising 58,585 bytes, submitted concurrently with the filing of this application is incorporated herein by reference.

### FIELD AND BACKGROUND OF THE INVENTION

The present invention, in some embodiments thereof, relates to methods of analyzing the antimicrobial peptide (AMP) profile of a tissue to obtain information regarding physiological states of the tissue, to AMP proxies for obtaining that information and for diagnosing and monitoring disease.

A beneficial balance between the mammalian host and its gut microbiome is critical for host homeostasis and host-microbe mutualism. Disruption of such balanced microbial composition and its collective functions, termed “dysbiosis”, has been associated with a multitude of intestinal and extra-intestinal health-related outcomes in humans, including inflammatory bowel disease (IBD), autoimmune disease, cardiometabolic disease, cancer and neurodegenerative disorders. Complex host-microbiome interactomes, orchestrating indigenous and dysbiotic microbiome colonization, entrenchment, and its pathophysiological consequences, are increasingly studied using metagenomic and metabolomic pipelines. However, elucidation of the vast and potentially bioactive repertoire of proteins and peptides associated with the gut microbiome, and their relation to disease remains elusive.

An important component of this vast repertoire of the host-derived proteome includes antimicrobial peptides (AMPs), a diverse group of evolutionarily conserved short defense proteins and peptides produced by various host cells in plants, invertebrates, amphibians, and mammals. Examples of well-characterized mammalian AMP families include lysozymes, cathelicidins, alpha- and beta-defensins, and regenerating islet-derived (Reg) proteins. The expression, secretion and activity of this diverse gut AMP repertoire in both epithelial and immune cells may be regulated by the commensal microbiome. Some AMPs constitutively shape the indigenous commensal repertoire and microbiome ecology. Other AMPs are secreted in response to pathogenic infection. Some AMPs are active against a broad range of bacteria, while others are known to be active only against specific strains.

The collective impact of the AMP repertoire on gut homeostasis in health and in microbiome-associated disease, and its potential diagnostic and therapeutic use remain elusive due to several key limitations. First, the vast majority of AMP-focused studies rely only on transcriptional activity of AMP expression while disregarding translational regulation, protein turn-over, and actual AMP levels following intestinal secretion. Second, most studies focus on decoding functions of individual AMPs or AMP families, while study of the complex AMP landscape and its potential impact on the microbiome in health and disease remains at its infancy. Third, the downstream impact of this global AMP landscape on commensal or pathogen colonization, de-novo development of disease-associated dysbiosis and disease manifestations remains poorly understood.

Likewise, reliable AMP proxies for assessing tissue health have not been available, due to the paucity and partial nature of the data surrounding the AMP landscape in health and disease.

Background art includes Zhong et al (2019), Leshem et al (2020), US Patent Application Publication No. 20110212104 to Beaumont et al, US Patent Application Publication No. 20210024997 to Shalek et al, Kang et al (2019) and Grant et al (2019).

### SUMMARY OF THE INVENTION

According to an aspect of some embodiments of the present invention there is provided a method of determining a physiological state of a tissue of a subject, the method comprising:

measuring the levels of a plurality of antimicrobial proteins or peptides (AMPs) in a sample of a secretion, exudate or excretion of the tissue of the subject, wherein the plurality of AMPs is predominantly AMPs having shared abundance in the tissue and the secretion, exudate or excretion of the tissue in a predetermined physiological state and wherein a statistically significant correlation between the levels of the plurality of AMPs in the secretion, exudate or excretion and the levels of the plurality of AMPs in the tissue in the physiological state is identified, determining the presence of the physiological state of the tissue, and wherein levels of the plurality of AMPs constitutes a proxy for the physiological state of the tissue.

According to some embodiments of the invention the AMPs comprise:
a) annotated antimicrobial proteins and peptides, and
b) proteins and peptides having at least 2 out of the following criteria:
i) secreted protein or peptide;
ii) protein or peptide having two or more of amino acids arginine, lysine or histidine, having a net charge between −5 and +10, having hydrophobic content between 10% and 80% and less than 200 amino acids in length;
iii) comprising a bacterial ligand-binding domain;
iv) having an enhancing antibacterial defense response activity;
v) having immunomodulatory activity, and
vi) having a tissue distribution pattern similar to AMP disclosed in an antimicrobial protein and peptide database upon commensal colonization, pathogenic infection or intestinal inflammation.

According to an aspect of some embodiments of the present invention there is provided a method of determining a proxy for a physiological state of a tissue, the method comprising: (a) determining an antimicrobial protein and peptide (AMP) profile of a sample of the tissue, (b) determining the AMP profile of a sample of an exudate, secretion or excretion of the tissue and (c) identifying a plurality of AMPs having shared abundance in the tissue and the exudate, secretion or excretion in a given physiological state, wherein the plurality of AMPs having shared abundance constitutes the proxy.

According to some embodiments of the invention, the physiological state of the tissue is selected from the group consisting of inflammation, infection, autoimmune disease and metabolic disease.

According to some embodiments of the invention, the physiological state of the tissue is dysbiosis.

According to some embodiments of the invention, the annotated AMPs are from at least one database selected from the group consisting of Antimicrobial Peptide Database (APD), Data Repository of Antimicrobial Peptides (DRAMP 2.0), Database of Antimicrobial Activity and Structure of Peptides (DBAASP) and Collection of antimicrobial peptides (CAMP).

According to some embodiments of the invention, the bacterial ligand-binding domain binds bacterial lipopolysaccharides (LPS), bacterial lipoteichoic acids (LTA), bacterial lectins, and/or peptidoglycan precursor (Lipid II).

According to some embodiments of the invention the antibacterial defense response activity is selected from the group consisting of phagocytosis, bacterial membrane pore formation, radical oxygen species (ROS) production, hydrolytic enzyme release, limitation of free nutrient/inorganic-ions and promotion of growth of competing bacterial species.

According to some embodiments of the invention, the immunomodulatory activity is selected from the group consisting of chemoattraction of monocytes, neutrophils, dendritic cells, and T-cells, chemokine and cytokine production and neutrophil degranulation.

According to some embodiments of the invention, the tissue is gut mucosa and the excretion is feces. According to other embodiments, the tissue is lung and the secretion is alveolar fluid.

According to some embodiments of the invention, the tissue is vaginal, cervical or uterine tissue and the secretion and/or exudate is vaginal fluid.

According to some embodiments of the invention, the tissue is oral cavity tissue and the secretion is saliva.

According to some embodiments of the invention, the tissue is ocular tissue and the secretion is lacrimal fluid or tears.

According to some embodiments of the invention, the tissue is genitourinary tissue and the excretion is urine.

According to some embodiments of the invention, determining the levels of the plurality of AMPs is effected on a protein extract of the sample of the secretion, exudate or excretion.

According to some embodiments of the invention, the sample of a secretion, exudate or excretion is depleted of membrane proteins and membrane-bound organelle proteins.

According to some embodiments of the invention, the tissue is selected from the group consisting of gut tissue, vaginal/cervical/uterine tissue, nasopharynx, lung tissue, genitourinary tissue, ocular tissue and skin.

According to some embodiments of the invention, the tissue is selected from the group consisting of connective tissue, muscle, nervous tissue and epithelial tissue.

According to some embodiments of the invention, the physiological state of the tissue is affected by a condition selected from the group consisting of inflammation, infection, auto-immune disease and metabolic disorders.

According to some embodiments of the invention, when the tissue is human gut tissue and the secretion, exudate or excretion is stool, the physiological state is IBD, the AMPs are selected from the group consisting of PRTN3, AZU, S100A8, S100A9, S100A12, CTSG, LTF, PRB3, PGC, APCS, PGLYRP1, MPO, PRSS2, RNASE2, ELANE, ORM1, PLA2G1B, SOD2 and LGALS4.

According to some embodiments of the invention, when the tissue is human gut tissue and the secretion, exudate or excretion is stool, the physiological state is IBD, the AMPs are selected from the group consisting of AMPs having an amino acid sequence as set forth in SEQ ID Nos. 28-47.

According to some embodiments of the invention, the tissue is human gut tissue and the secretion, exudate or excretion is stool, the physiological state is Primary Sclerosing Cholangitis, and wherein the AMPs are selected from the group consisting of S100A8, S100A9, CEACAM8, ECP2, AZU, CTSG, PERM, Alpha-2-antichymotrypsin, PRTN3, EXP, RNAS4, ELANE, CSTA, Orosomucoid, Alpha Amylase 1 and MUC12.

According to some embodiments of the invention, the physiological state of the tissue is affected by dysbiosis.

According to some embodiments of the invention, the AMPs are identified by liquid chromatography, mass spectrometry or a combination of liquid chromatography and mass spectrometry.

According to some embodiments of the invention, the AMPs are identified by mass spectrometry-based label free quantification (LFQ).

According to some embodiments of the invention, the AMPs are identified by AMP binding moieties.

According to some embodiments of the invention, the AMP binding moieties are immobilized on an array.

According to an aspect of some embodiments of the present invention there is provided a kit for determining the physiological state of a tissue, comprising greater than 10 and fewer than 100 AMP-binding moieties, each binding moiety binding to a different AMP having shared abundance in the tissue and in an exudate, secretion or excretion of the tissue in a given physiological state.

According to some embodiments of the invention the binding moieties are selected from the group consisting of antibodies, aptamers and ligands of the greater than 10 and fewer than 100 AMPs.

According to some embodiments of the invention the tissue is gut tissue and the binding moieties are binding moieties binding to at least 10 of and fewer than 100 of the AMPs of Table S2.

According to some embodiments of the invention the tissue is gut tissue and the binding moieties are binding moieties binding to at least 10 of and fewer than 100 of the human orthologs of the murine AMPs of Table S2.

According to some embodiments of the invention the tissue is human gut tissue and the secretion, exudate or excretion is stool, the physiological state is IBD, and the AMPs are selected from the group consisting of PRTN3, AZU, S100A8, S100A9, S100A12, CTSG, LTF, PRB3, PGC, CP, APCS, PGLYRP1, MPO, PRSS2, RNASE2, ELANE, ORM1, PLA2G1B, SOD2 and LGALS4.

According to some embodiments of the invention, the tissue is human gut tissue and the secretion, exudate or excretion is stool, the physiological state is Primary Sclerosing Cholangitis, and wherein the AMPs are selected from the group consisting of S100A8, S100A9, CEACAM8, ECP2, AZU, CTSG, PERM, Alpha-2-antichymotrypsin, PRTN3, EXP, RNAS4, ELANE, CSTA, Orosomucoid, Alpha Amylase 1 and MUC12.

According to some embodiments of the invention the tissue is human gut tissue and the secretion, exudate or excretion is stool, the physiological state is IBD, and the AMPs are selected from the group consisting of AMPs having an amino acid sequence as set forth in SEQ ID Nos. 28-47.

According to an aspect of some embodiments of the present invention there is provided at least one tissue-associated AMP for use in treating or preventing an undesirable physiological state in a tissue of a subject.

According to some embodiments of the invention the tissue is gut tissue and the at least one tissue-associate AMP for use is a gut-associated AMP selected from the group consisting of Camp, Lnc2, Ltf, Mpo, Ngp, Prtn 3, Elane, Ctsg, Pglyrp1, S100a8, S100a9, Ear2, Epx, Prg2, Pla2g 1b, Reg3a, Reg3g and Reg4.

According to some embodiments of the invention the subject is a human and the gut associated AMP is the human ortholog of the murine gut-associated AMP selected from the group consisting of Camp, Lnc2, Ltf, Mpo, Ngp, Prtn 3, Elane, Ctsg, Pglyrp1, S100a8, S100a9, Ear2, Epx, Prg2, Pla2g 1b, Reg3a, Reg3g, Reg4.

According to some embodiments of the invention the tissue is gut tissue and said physiological state is dysbiosis.

According to some embodiments of the invention the tissue is gut tissue and the physiological state is IBD.

According to some embodiments of the invention the tissue is human gut tissue, and the gut-associated AMP is selected from the group consisting of PRTN3, AZU, S100A8, S100A9, S100A12, CTSG, LTF, PRB3, PGC, CP, APCS, PGLYRP1, MPO, PRSS2, RNASE2, ELANE, ORM1, PLA2G1B, SOD2 and LGALS4.

According to some embodiments of the invention, the tissue is gut tissue and the physiological state is Primary Sclerosing Cholangiatis.

According to some embodiments of the invention, the tissue is gut tissue and the gut-associatedAMPs are selected from the group consisting of S100A8, S100A9, CEACAM8, ECP2, AZU, CTSG, PERM, Alpha-2-antichymotrypsin, PRTN3, EXP, RNAS4, ELANE, CSTA, Orosomucoid, Alpha Amylase 1 and MUC12.

According to some embodiments of the invention the tissue is human gut tissue, and the gut-associated AMP is selected from the group consisting of AMPs having an amino acid sequence as set forth in SEQ ID Nos. 28-47.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1J illustrate the identification of an antimicrobial peptide (AMP) landscape along the murine gastrointestinal tract in colonized (SPF) and germ-free (GF) mice. (1A-1D) Comparison of AMP signatures in the ileum, cecum and distal colon between germ-free (GF) and specific-pathogen-free (SPF) mice (n=5 in each group). (1A) Principal component analysis (PCA) plot of AMPs derived from discovery proteomics. The barplots in the inset show the comparison of GF-SPF pairwise differences among the ileum, cecum and colon. **p<0.01, ***p<0.001 determined by unpaired Student's t-test. (1B) Venn diagram depicting shared and unique differentially abundant AMPs in SPF mice compared to GF mice in the ileum, cecum and distal colon. (1C-1D) Volcano plots showing differentially abundant proteins of SPF mice compared to GF mice in the ileum (1C) and cecum (1D) based on discovery proteomics. Light red circles indicate proteins with an adjusted p-value <0.10 by Mann—Whitney U test; red circles represent differentially abundant AMPs; top AMP hits are highlighted. (1E-1J) Ileal AMP landscape dynamics during mono-colonization of GF mice with mucosa-adherent versus non-adherent commensal segmented filamentous bacteria (SFB). (1E) Experimental design. GF C57BL/6 mice were mono-colonized with SFB indigenous to mice (mSFB) or rats (rSFB) for two weeks, or orally inoculated with sterile PBS−/− as a negative control (n=9-10 in each group). The terminal ileum mucosa was harvested and discovery proteomic analysis was performed. (1F) Volcano plot showing differentially abundant proteins upon mSFB mono-colonization in the terminal ileum compared to that of PBS-inoculated GF mice based on discovery proteomic analysis (light red circles indicate proteins with an adjusted p-value <0.05 by Mann—Whitney U test; red circles represent differentially abundant AMPs; top AMP hits are highlighted). (1G) PCA plot of AMPs derived from discovery proteomics. (1H) Isoclines fitted by a generalized additive model (GAM) to the ordination space of ileal AMPs showing the association between the ileal SFB abundance and the AMP landscape configuration. The isoclines represent predicted SFB abundance values, and the color gradient indicates the actual SFB abundance values. Pseudo R\hat{ }2 and p-value are attached. (1I) Top 10 AMPs significantly correlated with ileal SFB abundance based on Spearman's rank correlation coefficient (Rho). (1J) Spearman correlation analysis between the abundance of terminal ileum SFB and individual AMPs determined by discovery proteomics. The x-axis represents the log 10-transformed LFQ intensity of individual AMPs, and the y-axis represents SFB relative abundance determined by shotgun metagenomics sequencing.

FIGS. 2A-2N show the distal colonic and fecal AMP landscape dynamics during infection with Citrobacter rodentium in SPF mice. (2A) SPF C57BL/6 mice were orally infected with C. rodentium containing 1×10⁹ colony-forming units (CFU). Colon mucosa and stool samples were collected at day 0 (before infection), day 7 (peak of infection) and day 14 (recovery phase) (n=12 in each time point). Both discovery and targeted (AMP-focused) proteomic analysis and 16S rRNA gene sequencing were performed. (2B) Colonic bacterial composition at day 0, day 7 and day 14 based on sequencing of the 16s rRNA gene. (2C) Colonic mucosa-attached C. rodentium abundance at day 0, 7 and 14. TSS: Total Sum Scaling normalization. ***p<0.001 determined by Mann-Whitney U test. (2D) Volcano plot depicting differentially abundant proteins in distal colon at day 7 compared to day 0 based on discovery proteomics (light red circles indicate proteins with an adjusted p-value <0.05 by Mann—Whitney U test; red circles represent differentially abundant AMPs; top AMP hits are highlighted). (2E) PCA plot of colonic AMPs derived from discovery proteomics. (2F) Heatmap featuring differentially abundant distal colon mucosal AMPs at day 0, 7 and 14 (adjusted p-value <0.05 by Kruskal-Wallis rank-sum test). The color scale indicates Z-score of AMP intensity. (2G) Isoclines fitted by a generalized additive model (GAM) to the ordination space of colonic AMPs showing the association between the colonic C. rodentium abundance and the AMP landscape configuration. The isoclines represent predicted colonic C. rodentium abundance values, and the color gradient shows the actual C. rodentium abundance values. Pseudo R\hat{ }2 and p-value are attached. (2H) Top 12 AMPs significantly correlated with colonic C. rodentium abundance based on Spearman's rank correlation coefficient (absolute Rho>0.6). (2I) PCA plot of stool AMPs derived from targeted proteomics. (2J) Isoclines fitted by a generalized additive model (GAM) to the ordination space of stool AMPs showing the association between the stool C. rodentium abundance and the AMP landscape configuration. The isoclines represent predicted stool C. rodentium abundance values (log 10 transformed), and the color gradient shows the actual stool C. rodentium abundance values (log 10 transformed). Pseudo R\hat{ }2 and p-value are attached. (2K) Top 16 fecal AMPs derived from targeted analysis, which are significantly correlated with stool C. rodentium abundance based on Spearman's rank correlation coefficient (absolute Rho>0.6). (2L) Procrustes error plot illustrating the degree of match between colonic and fecal AMP signatures. The colonic AMP signature (circles) represents the target configuration while the fecal AMP signature (triangles) represents the rotated configuration. (2M) Venn diagram depicting shared and unique differentially abundant AMPs in infected colon mucosa compared to stool at day 7 of infection relative to day 0 (adjusted p-value <0.05 by Mann—Whitney U test). (2N) Heatmaps showing the shared differentially abundant AMPs detected in both colonic mucosa and stool at day 0, 7, 14 of infection. The color scale indicates median Z-score of AMP intensity.

FIGS. 3A-3J show that proteomics is superior to RNA-sequencing in characterizing the host AMP landscape during intestinal commensal colonization and pathogenic infection. (3A) PCA plot of terminal ileal mucosal transcriptome (RNA-Seq) of GF mice inoculated with PBS (control), rSFB or mSFB. (3B) Venn diagram depicting shared and unique differentially expressed terminal ileal transcripts (adjusted p-value <0.05) upon mSFB mono-colonization compared to PBS- or rSFB-mono-inoculated GF mice. (3C) Volcano plot demonstrating differentially expressed terminal ileal transcripts upon mSFB mono-colonization compared to that of PBS-inoculated GF mice (red circles represent genes with an adjusted p-value <0.05; the top hits are highlighted). (3D-3E) Venn diagram (3D) and heatmap (3E) depicting shared and unique differentially abundant terminal ileum protein level AMPs (discovery proteomics, adjusted p-value <0.05) and differentially expressed AMP transcripts (RNA-seq) in mSFB mono-colonized versus vehicle control GF mice. Shared AMPs (Reg3b and Reg3g) are highlighted in red. The color scale indicates Z-score of AMP intensity (left panel) or RNA-seq normalized read counts (right panel). (3F) PCA plot demonstrating the distal colonic mucosal transcriptome (RNA-seq) in SPF mice at day 0, 7 and 14 post C. rodentium infection. (3G) Venn diagram depicting shared and unique differentially expressed transcripts (adjusted p-value <0.05) in the distal colonic mucosa of infected mice at day 7 and day 14 post-infection, compared to day 0. (3H) Volcano plot featuring differentially expressed distal colonic mucosal transcripts at day 7 post-infection compared to day 0 (red circles represent genes with an adjusted p-value <0.05; the top hits are highlighted). (3I-3J) Venn diagram (3I) and heatmaps (3J) depicting shared and unique differentially abundant distal colonic mucosal AMPs (discovery proteomics, adjusted p-value <0.05) and differentially expressed AMP transcripts (RNA-seq) in the distal colon at day 7 post infection compared to day 0. Shared AMPs are highlighted in red. Representative AMPs that are only significantly differentially abundant at the protein level or transcriptional level are marked in blue. The color scale indicates Z-score of AMP intensity (left panel) or RNA-seq normalized read counts (right panel).

FIGS. 4A-4L show the fecal AMP landscape and microbiome dynamics in the murine acute dextran sodium sulfate (DSS)-induced colitis model. (4A) Experimental design and average weight change along disease course. SPF C57BL/6 mice were ad libitum administered 2% DSS in drinking water for 7 days, followed by resumption of regular water consumption. Stool samples were collected at day 0 (before exposure to DSS), day 3 & 5 (early inflammatory phase), day 16 (late inflammatory phase) and day 31 (end of weight recovery phase) (n=8-11 in each time point). (4B-4C) Volcano plot demonstrating differentially abundant fecal proteins, assessed by discovery proteomics, between disease phases at day 5 (4B) and day 16 (4C) after initiation of DSS consumption, compared to day 0 (light red circles indicate proteins with an adjusted p-value <0.05 by Wilcoxon signed-rank test; red circles represent differentially abundant AMPs; top AMP hits are highlighted). “Defa11*” represents the AMP group “alpha-defensins 11, 24, 15, 9, 6”; “Defa17*” represents “alpha-defensins 17, 1”. (4D) PCA plot of stool AMPs quantified at different disease phases by discovery proteomics. (4E) Heatmap depicting differentially abundant fecal AMPs at day 0, 3, 5, 16 and 31 following initiation of DSS consumption (adjusted p-value <0.05 by one-way repeated measures ANOVA). The color scale indicates Z-score of AMP intensity. (4F) Comparison of fecal content of lymphocyte antigen 6 complex locus G6D (Ly6G, a neutrophil marker) and mucin-2 (Muc2, a goblet cell marker), detected by discovery proteomics at day 0, 3 and 5 following initiation of DSS consumption. *p<0.05, ***p<0.001 determined by Wilcoxon signed-rank test. (4G-4H) Heatmaps depicting the changes of fecal AMPs produced by polymorphonuclear leukocytes (PMN) (4G) and intestinal epithelial cells (IEC) (4H) at day 0, 3, 5, 16, and 31 following initiation of DSS consumption. The color scale indicates Z-score of AMP intensity. “Defa11*” represents the AMP group “alpha-defensins 11, 24, 15, 9, 6”; “Defa17*” represents “alpha-defensins 17, 1”; “Defa8*” represents “alpha-defensins 8, 16, 7, 3, 2, 13”; “Defa21*” represents “alpha-defensins 21, 22”. (4I) Linear regression analysis regressing the area under the curve (AUC) of weight loss against the intra-individual Euclidean distance of AMP abundance (discovery proteomics) between day 5 following initiation of DSS consumption and baseline (day 0). (4J) PCA plot of stool microbiome composition (after centered log-ratio transformation) based on shotgun metagenomic sequencing. (4K) Fecal bacterial genus level dynamics (shotgun metagenomic sequencing) at day 0, 3, 5, 16 and 31 following initiation of DSS consumption. (4L) Procrustes error plot illustrating the degree of match between the AMP and the microbiome signatures. The fecal microbiome composition (circles) represents the target configuration while the fecal AMP signature (triangles) represents the rotated configuration.

FIGS. 5A-5G show how the AMP landscape can be predictive of microbial dynamics and disease severity in the murine model of colonic inflammation. (5A-5B) Prediction of DSS colitis phases (day 0, day 3, day 5, day 16, day 31) by stool AMP landscape (5A) and fecal microbiome composition (5B) using multiclass support vector machine (SVM) classifier with a linear kernel applying leave one cage out cross-validation scheme. Confusion matrices summarize the classification performance with accuracy. (5C-5E) Prediction of colitis severity for each mouse (quantified by the AUC of weight loss) upon DSS consumption, by stool AMP landscape (5C), fecal microbiome composition (5D) and PCA-based fecal microbiome configuration (5E) using a gradient boosting regression model applying leave one cage out cross-validation scheme. The performance is summarized by scatter plots of observed versus predicted weight loss. MSE: mean squared error. (5F-5G) Correlation matrix depicting selected pairs of fecal bacterial species abundances (metagenomic sequencing) (5F) and microbiome functional pathways (metagenomic sequencing-based KEGG pathways) (5G) predicted by AMP features employing sparse partial least squares (sPLS) regression (absolute threshold >0.7 are shown). “Defa17*” represents the AMP group “alpha-defensins 17, 11”; “Defa8*” represents “alpha-defensins 8, 16, 7, 3, 2, 13”.

FIGS. 6A-6I depict the fecal proteomic AMP signature in human IBD. (6A) Overview of the human cohort design. We collected and performed stool tandem proteomic and metagenomic sequencing from a human IBD cohort: 26 pediatric Crohn's disease (CD) patients (including inactive and active disease status) and 28 healthy controls (HC); (6B) Volcano plot showing differentially abundant proteins in stool samples of CD patients compared to HC (light red circles indicate proteins with an adjusted p-value <0.10 by Mann—Whitney U test; red circles represent differentially abundant AMPs; top AMP hits are highlighted). (6C) PCA plot of AMPs derived from discovery proteomics. (6D) Heatmap featuring differentially abundant AMPs in stool samples of CD patients compared to HC (adjusted p-value <0.10 by Mann—Whitney U test). Selected AMPs are labeled. The color scale indicates Z-score of AMP intensity. (6E) PCA plot of stool microbiome composition (after centered log-ratio transformation) based on shotgun metagenomic sequencing. (6F) Volcano plot showing differentially abundant fecal bacterial species between CD patients and healthy controls (red circles indicate an adjusted p-value <0.10 by Mann—Whitney U test; top hits are highlighted). (6G) Procrustes error plot illustrating the degree of match between the AMP and the microbiome signatures. The fecal microbiome composition (circles) represents the target configuration, while the fecal AMP signature (triangles) represents the rotated configuration. (6H) Correlation matrix depicting selected pairs of fecal bacterial species abundances (metagenomic sequencing) predicted by AMP features employing sparse partial least squares (sPLS) regression (absolute threshold >0.35 are shown). (6I) Classification of CD versus healthy status by stool AMP landscape (red line), fecal microbiome composition (blue line), and combined signatures (green line) applying sigmoid kernel SVM classifiers by a stratified 5-fold cross-validation. Receiver operating characteristic (ROC) curves summarize the trade-offs between SVM classifier true and false-positive rates.

FIGS. 7A-7H details the identification of the antimicrobial peptide (AMP) landscape along the murine gastrointestinal tract in colonized and germ-free mice. (7A) Workflow of the metagenomics—proteomics pipeline. Ileum, cecum, distal colon and stool samples were harvested from naïve mice. DNA was subjected to metagenomics sequencing, while the soluble protein content was subjected to mass spectrometry-based discovery proteomics, including characterization of the AMP landscape. Stool samples were also subjected to a custom-designed mass spectrometry-based targeted proteomics. The AMP landscape and microbiome composition were correlated, while machine learning models were employed to predict disease phenotypes by AMP signatures. (7B) Principal component analysis (PCA) plot of discovery proteomics. The barplots in the inset show the comparison of GF-SPF pairwise differences among the ileum, cecum, and colon. **p<0.01, ***p<0.001 determined by unpaired Student's t-test. (7C-7E) Volcano plots showing differentially abundant proteins of SPF mice in the ileum compared to the colon (7C), in the ileum compared to the cecum (7D), and in the cecum compared to the colon (7E). Light red circles indicate proteins with an adjusted p-value <0.10 by Mann—Whitney U test; red circles represent differentially abundant AMPs; top AMP hits are highlighted. (7F-7H) Protein set enrichment analysis for AMP signature proteins of SPF mice in the ileum compared to the colon (7F), in the ileum compared to the cecum (7G), and in the cecum compared to the colon (7H).

FIGS. 8A-8F further details the comparison of the antimicrobial peptide (AMP) landscape along the murine gastrointestinal tract in colonized and germ-free mice. (8A) Venn diagram of shared and unique differentially abundant proteins in SPF mice compared to GF mice in the ileum, cecum and distal colon. (8B-8D) Protein set enrichment analysis for AMP signature proteins of SPF mice compared to GF mice in the ileum (8B), cecum (8C) and distal colon (8D). (8E) Clustered heatmap depicting the abundance of AMPs in the ileum, cecum and distal colon in GF and SPF mice. The color scale indicates Z-score of AMP intensity. (8F) Volcano plot showing differentially abundant proteins of SPF mice compared to GF mice in the colon based on discovery proteomics. Light red circles indicate proteins with an adjusted p-value <0.10 by Mann-Whitney U test; red circles represent differentially abundant AMPs; top AMP hits are highlighted.

FIGS. 9A-9I details the ileal AMP landscape dynamics during mono-colonization of GF mice with mucosa-adherent versus non-adherent commensal segmented filamentous bacteria (SFB). (9A) SFB relative abundance in terminal ileum of GF mice inoculated with PBS (control), rSFB or mSFB determined by metagenomic sequencing. ***p<0.001 determined by unpaired Mann-Whitney U test. (9B) PCA plot of the discovery proteomics. (9C) Volcano plot showing differentially abundant proteins in terminal ileum of rSFB-inoculated GF mice compared to PBS-inoculated GF mice based on discovery proteomics. (9D) Volcano plot showing differentially abundant proteins upon mSFB mono-colonization in terminal ileum compared to that of rSFB-inoculated GF mice based on discovery proteomics (light red circles indicate proteins with an adjusted p-value <0.05 by Mann—Whitney U test; red circles represent differentially abundant AMPs; top changing AMPs are highlighted). (9E) Volcano plot showing differentially abundant proteins upon mSFB mono-colonization in terminal ileum compared to that of PBS-inoculated GF mice based on discovery proteomics (red circles represent proteins with an adjusted p-value <0.05 by Mann-Whitney U test; proteins associated with interferon signature are highlighted). (9F) Heatmap featuring differentially abundant AMPs in the terminal ileum of GF mice inoculated with PBS (control), rSFB or mSFB (adjusted p-value <0.05 by Kruskal-Wallis rank-sum test). Selected AMPs are labeled. The color scale indicates Z-score of AMP intensity. (9G-9I) Shared and unique differentially abundant AMPs in terminal ileum upon mSFB mono-colonization compared to that of PBS- or rSFB-inoculated GF mice (adjusted p-value <0.05 by Mann-Whitney U test), displayed by volcano plots (9G-9H) and Venn diagram (9I).

FIGS. 10A-10F shows the distal colonic mucosal AMP landscape upon IL18 supplementation in GF mice. (10A) Experimental design. GF C57BL/6 mice were injected intraperitoneally with recombinant IL18 for 5 days or with sterile PBS−/− as the negative control. Distal colon samples were harvested for discovery proteomics. (10B) PCA plot of distal colonic discovery proteomics. (10C) Volcano plot depicting differentially abundant proteins in distal colon upon IL18 supplementation compared to that of PBS-injected GF mice based on discovery proteomics (red circles represent proteins with adjusted p-value <0.10 by Mann-Whitney U test; top changing AMPs are highlighted). (10D) Protein set enrichment analysis for AMP signature proteins in distal colon upon IL18 supplementation compared to that of PBS-injected GF mice. (10E) PCA plot of distal colonic AMPs derived from discovery proteomics. (10F) heatmap showing the differentially abundant AMPs (adjusted p-value <0.10 by Mann-Whitney U test) in distal colon upon IL18 supplementation compared to that of PBS-injected GF mice. The color scale indicates Z-score of AMP intensity.

FIGS. 11A-11N show the evolution of distal colonic and fecal AMP landscape dynamics during infection with Citrobacter rodentium in specific-pathogen-free (SPF) mice. (11A-11B) Proteomic landscape during C. rodentium infection in different GI locations. (11A) Experimental design. SPF C57BL/6 mice were infected orally with C. rodentium containing 1×10⁹ colony-forming units (CFU). Colon, cecum and ileum mucosa were collected on day 0 (before infection), day 3, and day 6 (after infection). (11B) Volcano plots showing differentially abundant proteins (red circles indicate an adjusted p-value <0.05 by Mann-Whitney U test) in colon, cecum and ileum mucosa at day 6 compared to day 0 based on discovery proteomics. (11C) PCA plot of the discovery proteomics of distal colon mucosa. (11D) Volcano plot showing differentially abundant proteins in distal colon at day 14 compared to day 0 based on discovery proteomics (light red circles indicate an adjusted p-value <0.05 by Mann-Whitney U test; red circles represent differentially abundant AMPs; top AMP hits are highlighted). (11E-11G) Venn diagram (11E) and volcano plots (11F-11G) showing all shared and unique differentially abundant AMPs (adjusted p-value <0.05 by Mann-Whitney U test) in distal colon at day 7 (11F) and day 14 (11G) compared to day 0. (H) Normalized stool C. rodentium abundance at day 0, 7, and 14. TSS: Total Sum Scaling normalization. ***p<0.001 determined by Mann-Whitney U test. (11I) Line plot showing changing patterns of fecal AMPs based on peptides quantified using stable isotope-labeled (SIL) peptides in a targeted proteomics assay. The y-axis represents log 2 fold change from baseline values of the calculated light/heavy ratios. (11J) Heatmap showing differentially abundant AMPs at day 0, 7 and 14 of infection in stool by targeted proteomics (adjusted p-value <0.05 by Kruskal-Wallis rank-sum test). The color scale indicates Z-score of AMP intensity. (11K-11M) Venn diagram (11K) and volcano plots (11L-11M) showing all shared and unique differentially abundant AMPs (adjusted p-value <0.05 by Mann-Whitney U test) in stool targeted proteomics at day 7 (11L) and day 14 (11M) compared to day 0. (11N) Spearman correlation between the intensity of representative AMPs (S100a9, Reg4) in the distal colonic mucosa and their corresponding fecal AMP intensity.

FIGS. 12A-12F are graphs showing fecal AMP-targeted proteomics during infection with Citrobacter rodentium in specific-pathogen-free (SPF) mice. (12A-12F) Bar plots demonstrating inter-individual changing patterns of representative AMPs (each AMP is represented by 2-3 different peptides) at day 0, 7, and 14. The y-axis represents the total peak fragment area extracted from the PRM assay (each color represents the area of a different fragment), and the x-axis displays different samples. The listed AMPs include (12A) protein S100-A9 (5100a9), (12B) cathelicidin antimicrobial peptide (12Camp), (12C) neutrophil gelatinase-associated lipocalin (Lcn2), (12D) neutrophil elastase (Elane), (12E) angiogenin-4 (Ang4) and (12F) regenerating islet-derived protein 4 (Reg4).

FIGS. 13A-13H show the superiority of proteomics over RNA-sequencing in characterizing the host AMP landscape during intestinal commensal colonization and pathogenic infection. (13A) Volcano plot showing differentially expressed AMP transcripts upon mSFB mono-colonization in the terminal ileum compared to PBS-inoculated GF mice (hollow circles represent genes encoding for AMPs, and only significantly differentially expressed AMP genes with adjusted p-value <0.05 are highlighted and marked in red). (13B) Volcano plot demonstrating differentially expressed terminal ileal transcripts upon mSFB mono-colonization compared to that of rSFB-inoculated GF mice (red circles represent genes with an adjusted p-value <0.05; the top hits are highlighted). (13C) Volcano plot showing differentially expressed AMP transcripts upon mSFB mono-colonization in the terminal ileum compared to that of rSFB-inoculated GF mice (hollow circles represent genes encoding for AMPs, and only significantly differentially expressed AMP genes with adjusted p-value <0.05 are highlighted and marked in red). (13D) Gene Ontology (GO) enrichment analysis of genes derived from differentially abundant proteins (discovery proteomics, left panel) and GO enrichment analysis of differentially upregulated genes (RNA-sequencing, right panel) upon mSFB mono-colonization in the terminal ileum compared to vehicle control GF mice. (13E) Volcano plot depicting differentially expressed distal colonic mucosal genes at day 14 post-infection compared to day 0 (red circles represent genes with an adjusted p-value <0.05; the top hits are highlighted). (13F-13G) Volcano plot showing the differentially expressed AMP transcripts in the distal colon at day 7 (F) and day 14 (13G) post-infection compared to day 0 (hollow circles represent genes encoding for AMPs; red circles represent differentially expressed AMP genes with an adjusted p-value <0.05; top hits are highlighted). (13H) GO enrichment analysis of genes derived from differentially abundant proteins (discovery proteomics, left panel) and GO enrichment analysis of differentially upregulated genes (RNA-sequencing, right panel) in the distal colon of infected mice (day 7) compared to uninfected mice (day 0).

FIGS. 14A-14L show the fecal AMP landscape and microbiome dynamics in the murine acute dextran sodium sulfate (DSS)-induced colitis model. (14A) Weight loss of each mouse along DSS treatment course until day 8 (n=11). (14B) PCA plot of stool discovery proteomics quantified at different disease phases. (14C) Representative histology images of the distal colon at day 0 and day 5. (14D) Comparison of live cell counts of lamina propria neutrophils (CD11b+Ly6G+MHCII−) and epithelial cells (CD45−EpCAM+) in the distal colon at day 0 and day 5 detected by flow cytometry. *p<0.05, ***p<0.001 determined by Wilcoxon signed-rank test. (14E-14F) Volcano plot demonstrating differentially abundant fecal AMPs between disease phases after initiation of DSS consumption, at day 5 (14E) and day 16 (14F) compared to day 0 (red circles indicate proteins with an adjusted p-value <0.05 by Wilcoxon signed-rank test; top AMP hits are highlighted). “Defa11*” represents the AMP group “alpha-defensins 11, 24, 15, 9, 6”; “Defa17*” represents “alpha-defensins 17, 1”. (14G-14H) Volcano plot showing differentially abundant fecal bacterial species between disease phases after initiation of DSS consumption, at day 5 (14G) and day 16 (14H) compared to day 0 (red circles indicate an absolute FC>2 and adjusted p-value <0.05 by Wilcoxon signed-rank test; top hits are highlighted). (14I-14J) PCA plot of stool microbiome function represented by KEGG orthologous (KO) genes (14I) and KEGG pathways (14J) (after centered log-ratio transformation) based on shotgun metagenomic sequencing. (14K) Volcano plot depicting differentially abundant fecal KEGG pathways between disease phases after initiation of DSS consumption, at day 5 compared to day 0 (red circles indicate an adjusted p-value <0.05 by Wilcoxon signed-rank test; top pathway hits are highlighted). (14L) Procrustes error plot illustrating the degree of match between the AMP and the microbiome signatures. The fecal microbiome function (circles) represents the target configuration, while the fecal AMP signature (triangles) represents the rotated configuration.

FIGS. 15A-15L show the validation of fecal AMP landscape and microbiome dynamics in an independent murine acute dextran sodium sulfate (DSS)-induced colitis cohort. (15A) Line plot showing the weight loss of each mouse the along DSS treatment course until day 8 (n=10). (15B) Volcano plot showing differentially abundant fecal proteins, assessed by discovery proteomics, between disease phases at day 5 compared to day 0 (light red circles indicate proteins with an adjusted p-value <0.05 by Wilcoxon signed-rank test; red circles represent differentially abundant AMPs; top AMP hits are highlighted). (15C) PCA plot of stool AMPs derived from discovery proteomics quantified at different disease phases. (15D) Heatmap depicting differentially abundant fecal AMPs in the stool at day 0, 3, and 5 following initiation of DSS consumption (adjusted p-value <0.05 by one-way repeated measures ANOVA). The color scale indicates Z-score of AMP intensity. (15E) Comparison of fecal content of lymphocyte antigen 6 complex locus G6D (Ly6G, a neutrophil marker) and mucin-2 (Muc2, a goblet cell marker) detected by discovery proteomics at day 0, 3, and 5 following initiation of DSS consumption. *p <0.05, ***p<0.001 determined by Wilcoxon signed-rank test. (15F-15G) Heatmaps showing the changes of fecal AMPs produced by PMN (15F) and IEC (15G) in the stool at day 0, 3, and 5 following initiation of DSS consumption. The color scale indicates Z-score of AMP intensity. “Defa11*” represents the AMP group “alpha-defensins 11, 24, 15, 9, 6”; “Defa17*” represents “alpha-defensins 17, 1”; “Defa8*” represents “alpha-defensins 8, 16, 7, 3, 2, 13”; “Defa21*” represents “alpha-defensins 21, 22”. (15H) PCA plot of the stool microbiome composition (after centered log-ratio transformation) based on shotgun metagenomic sequencing. (15I) Fecal bacterial genus level dynamics (shotgun metagenomic sequencing) at day 0, 3, and 5 following initiation of DSS consumption. (15J) PCA plot of stool microbiome function represented by KEGG pathways (after centered log-ratio transformation) based on shotgun metagenomic sequencing. (15K) Linear regression analysis between the area under the curve (AUC) of weight loss and the intra-individual Euclidean distance of AMP abundance (discovery proteomics) between day 5 following initiation of DSS consumption and baseline (day 0). (15L) Procrustes error plot illustrating the degree of match between the AMP and the microbiome signatures. The fecal microbiome composition (circles) represents the target configuration while the fecal AMP signature (triangles) represents the rotated configuration.

FIGS. 16A-16H details fecal AMP-targeted proteomics in an independent murine acute dextran sodium sulfate (DSS)-induced colitis cohort. (16A) PCA plot of stool AMPs derived from targeted proteomics. (16B) Heatmap depicting changes of all fecal AMPs at day 0, 3 and 5 following initiation of DSS consumption, based on fecal targeted proteomics. The color scale indicates Z-score of AMP intensity. (16C-H) Bar plots demonstrating inter-individual changing patterns of representative AMPs (each AMP is represented by 2-3 different peptides) at day 0, 3, and 5. The y-axis represents the total peak fragment area extracted from the PRM assay (each color represents the area of a different fragment), and the x-axis displays different samples. The listed AMPs include (16C) cathelicidin antimicrobial peptide (Camp), (16D) neutrophil gelatinase-associated lipocalin (16Lcn2) and (16E) neutrophilic granule protein (Ngp), (16F) phospholipase A2 (Pla2g1b), (16G) angiogenin-4 (Ang4) and (16H) alpha-defensin (Defa22).

FIGS. 17A-17G show the fecal proteomic AMP signature in human IBD. (17A) PCA plot of the discovery proteomics. (17B) Protein set enrichment analysis for AMP signature proteins in stool samples of Crohn's disease (CD) patients compared to healthy controls (HC). (17C) Comparison of Shannon diversity index in stool samples in CD patients and HC. **p<0.01 determined by Mann-Whitney U test. (17D) PCA plot of AMPs derived from discovery proteomics (comparing inactive and active disease status). (17E) PCA plot of stool microbiome composition (after centered log-ratio transformation) based on shotgun metagenomic sequencing (comparing inactive and active disease status). (17F) Protein set enrichment analysis for AMP signature proteins in CD patients with active disease compared to inactive disease status. (17G) Volcano plot showing differentially abundant proteins in CD patients with active disease compared to inactive disease status (light red circles indicate proteins with an unadjusted p-value <0.10 by Mann—Whitney U test; red circles represent differentially abundant AMPs).

FIGS. 18A-18F show a metaproteomics analysis of stool samples from a German ulcerative colitis (UC) cohort (N=100, upper row, 18A-18C) and an Israeli pediatric Crohn's disease (CD) cohort (N=54, lower row, 18D-18F). (18A) A Principal Component Analysis (PCA) of untargeted host proteomics in stool samples from 50 German UC patients and 50 matched HC. Every dot depicts a stool sample's proteome. Statistically significant (PERMANOVA, p<0.001) clustering of UC patients from controls suggest a global difference in the proteomic signature. (18B) A volcano plot depicting differentially abundant proteins between German UC patients and HC. Each dot depicts a protein. The x axis denotes the log transformed fold change between the level of a given protein between UC patients (enriched proteins are to the right of the y axis) and HC (enriched proteins are to the left of the y axis). The y axis denotes the log transformed q value for the statistical significance (Welsch t-test). P-value was corrected by false detection rate (FDR) for multiple comparisons. Red dots denote statistically significant peptides (including some AMPS). Grey dots denote peptides with a differential abundance that did not meet the statistical significance threshold. (18C) Pathway enrichment analysis of all statistically significant differentially abundant proteins. The most strongly enriched pathways are presented. X axis denotes the log transformed p-value for the enrichment (hypergeometric test) (18D) A PCA of untargeted host proteomics in stool samples from 28 Israeli CD patients and 26 matched Healthy Controls (HC). Every dot depicts a stool sample's proteome. Statistically significant (PERMANOVA, p<0.001) clustering of Crohn's Disease (CD) patients from controls suggest a global difference in the proteomic signature. (18E) A volcano plot depicting differentially abundant proteins between Israeli CD patients and HC. Each dot depicts a protein. The x axis denotes the log transformed fold change between the level of a given protein between CD patients (enriched proteins are to the right of the y axis) and HC (enriched proteins are to the left of the y axis). The y axis denotes the log transformed q value for the statistical significance (Welsch t-test). P-value was corrected by false detection rate (FDR) for multiple comparisons. Red dots denote statistically significant peptides (including some AMPS—some aren't labeled). Grey dots denote peptides with a differential abundance that did not meet the statistical significance threshold. (18F) Pathway enrichment analysis of all statistically significant differentially abundant proteins. Top enriched pathways are presented. X axis denotes the log transformed p-value for the enrichment (hypergeometric test).

FIGS. 19A-19F show a metaproteomics analysis of stool samples from a German (N=129, upper row) and an Israeli (N=42, lower row) cohort of primary sclerosing cholangitis (PSC) patients and healthy controls (HC). (19A) shows a principle component analysis (PCA) of untargeted host proteomics in stool samples from 65 German PSC patients and 64 matched HC. Every dot depicts a stool sample's proteome. Statistically significant (PERMANOVA, p<0.001) clustering of PSC patients from controls suggest a global difference in the proteomic signature. (19B) A volcano plot depicting differentially abundant proteins between German PSC patients and HC. Each dot depicts a protein. The x axis denotes the log transformed fold change between the level of a given protein between PSC patients (enriched proteins are to the right of the y axis) and HC (enriched proteins are to the left of the y axis). The y axis denotes the log transformed q value for the statistical significance (Welsch t-test). P-value was corrected by false detection rate (FDR) for multiple comparisons. Green dots denote statistically significant antimicrobial peptides (AMPs). Red dots denote statistically significant peptides without known antimicrobial activity. Grey dots denote peptides with a differential abundance that did not meet the statistical significance threshold. (19C) Top discriminating features based on a machine-learning random-forest classifier algorithm, trained on German PSC proteomics data, showing that AMPs are among the top disease-discriminatory features. (19D) A PCA of untargeted host proteomics in stool samples from 17 PSC Israeli patients and 25 matched HC. Every dot depicts a stool sample's proteome. Statistically significant (PERMANOVA, p<0.001) clustering of PSC patients from controls suggest a global difference in the proteomic signature. (19E) A volcano plot depicting differentially abundant proteins between Israeli PSC patients and HC. Each dot depicts a protein. The x axis denotes the log transformed fold change between the level of a given protein between PSC patients (proteins having greater expression in PSC patients are to the right of the y axis) and HC (proteins having greater expression in HC patients are to the left of the y axis). The y axis denotes the log transformed q value for the statistical significance (Welsch t-test). P-value was corrected by false detection rate (FDR) for multiple comparisons. Green dots denote statistically significant antimicrobial peptides (AMPs). Red dots denote statistically significant peptides without known antimicrobial activity. Grey dots denote peptides with a differential abundance that did not meet the statistical significance threshold. (19F) Top discriminating features based on a machine-learning random-forest classifier algorithm, trained on Israeli PSC proteomics data, showing that AMPs are among the top disease-discriminatory features.

### DESCRIPTION OF SPECIFIC EMBODIMENTS OF THE INVENTION

The present invention, in some embodiments thereof, relates to methods of analyzing the antimicrobial peptide (AMP) profile of a tissue to obtain information regarding physiological states of the tissue, to AMP proxies for obtaining that information and for diagnosing and monitoring disease.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The complexity of the internal environment has made it difficult to establish reliable correlations between host tissue health, the microbiome and the components of their interaction. For example, most studies of the GI host-microbiome interaction have analyzed host tissue gene expression patterns or levels of individual markers, but have not been able to account for the effects of the dynamics of GI physiology and the microbial ecosystem.

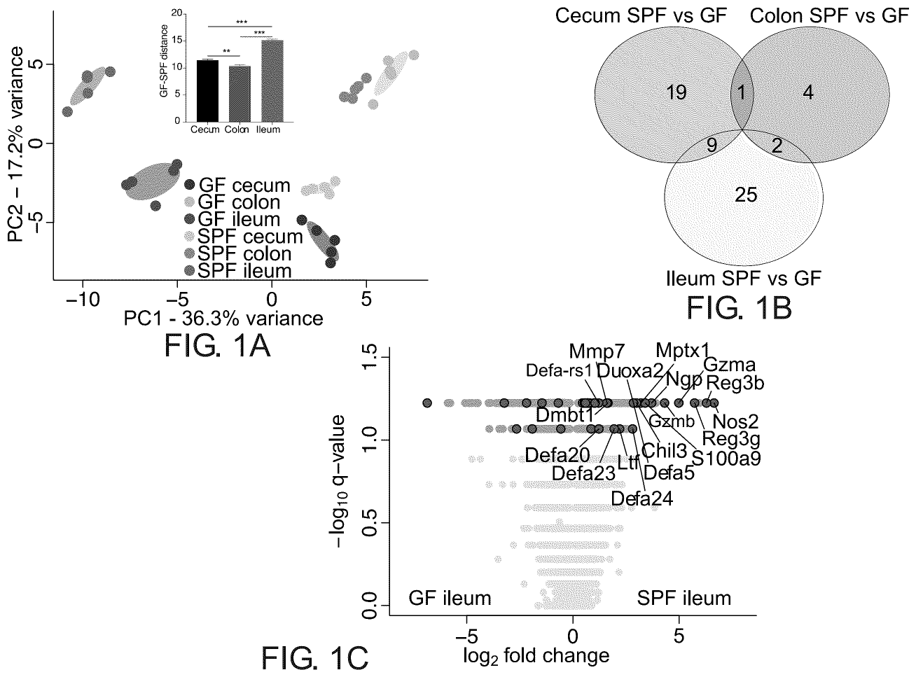

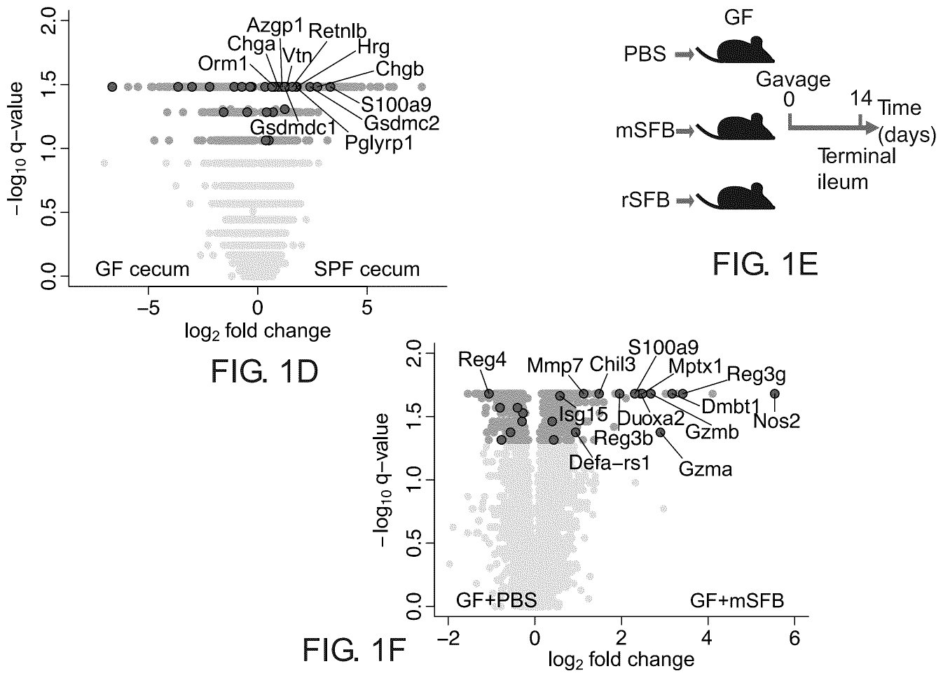

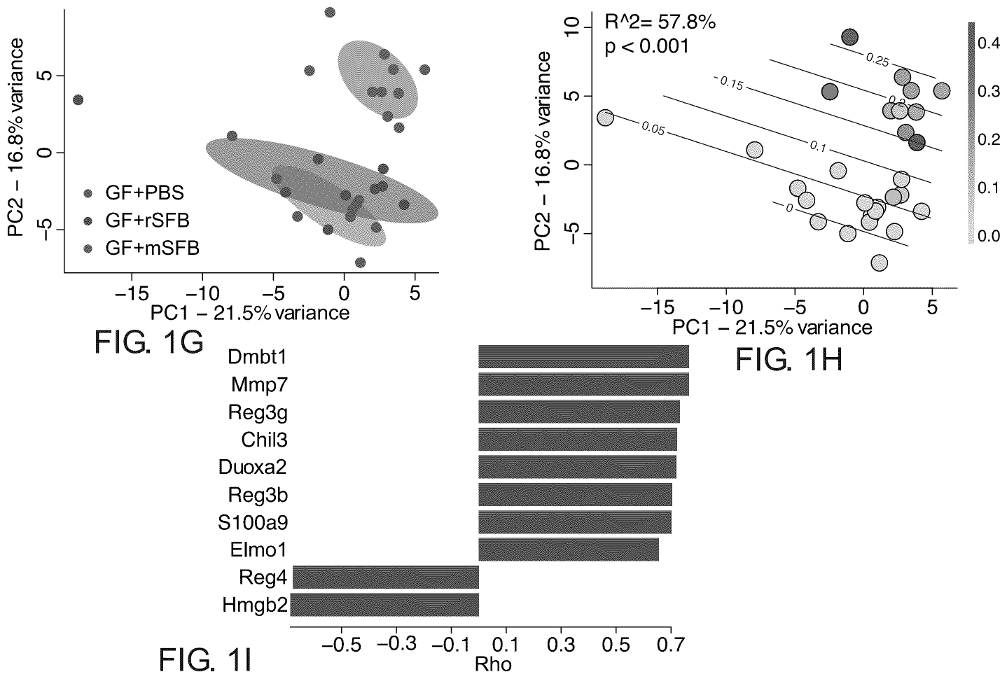

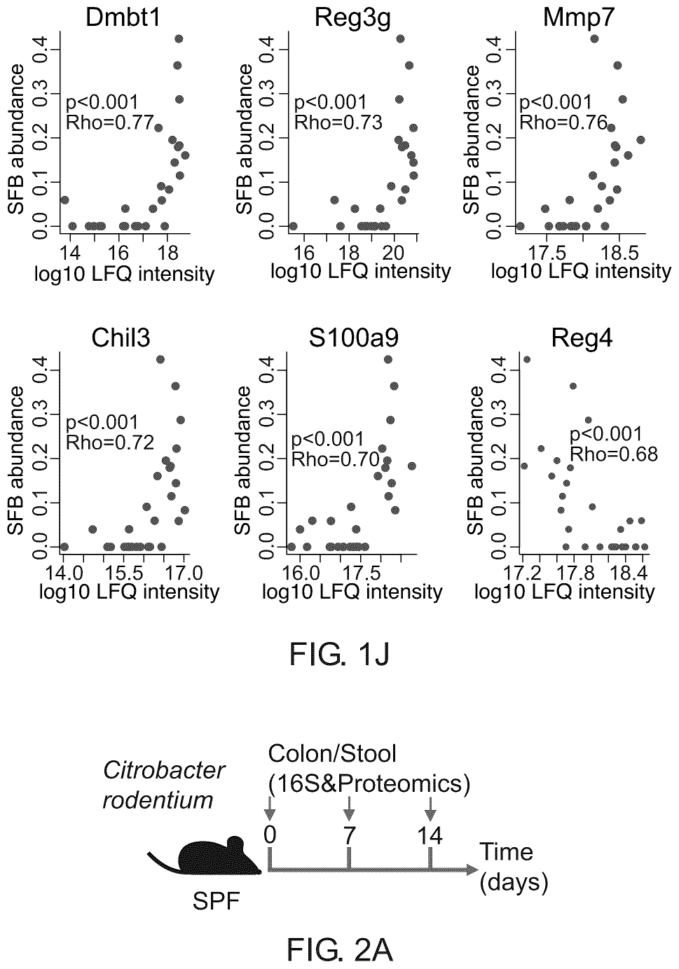

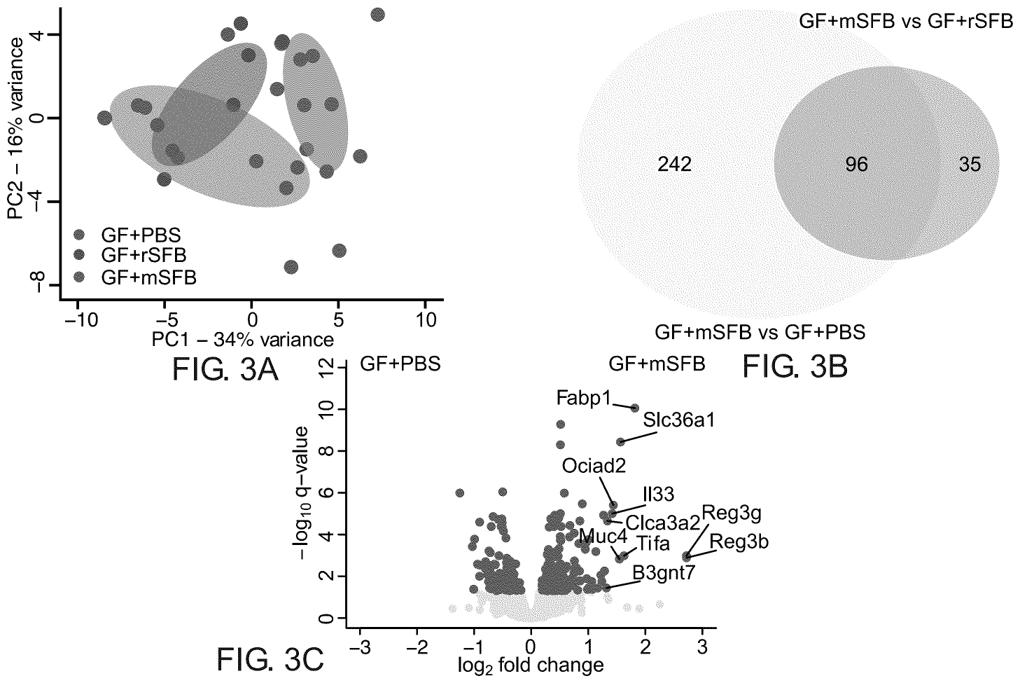

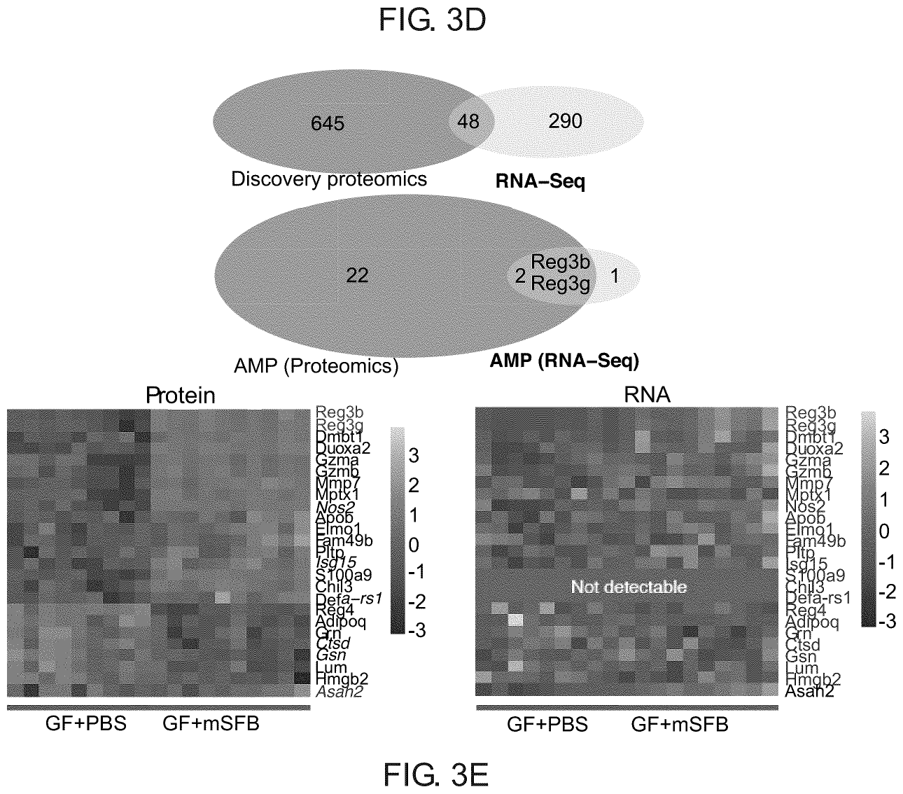

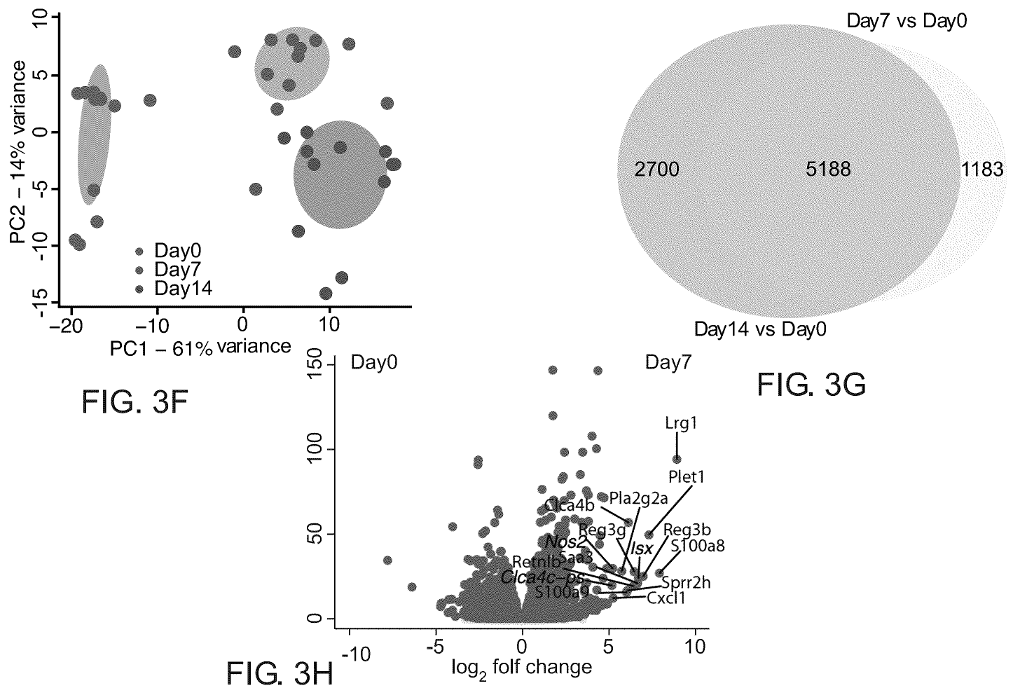

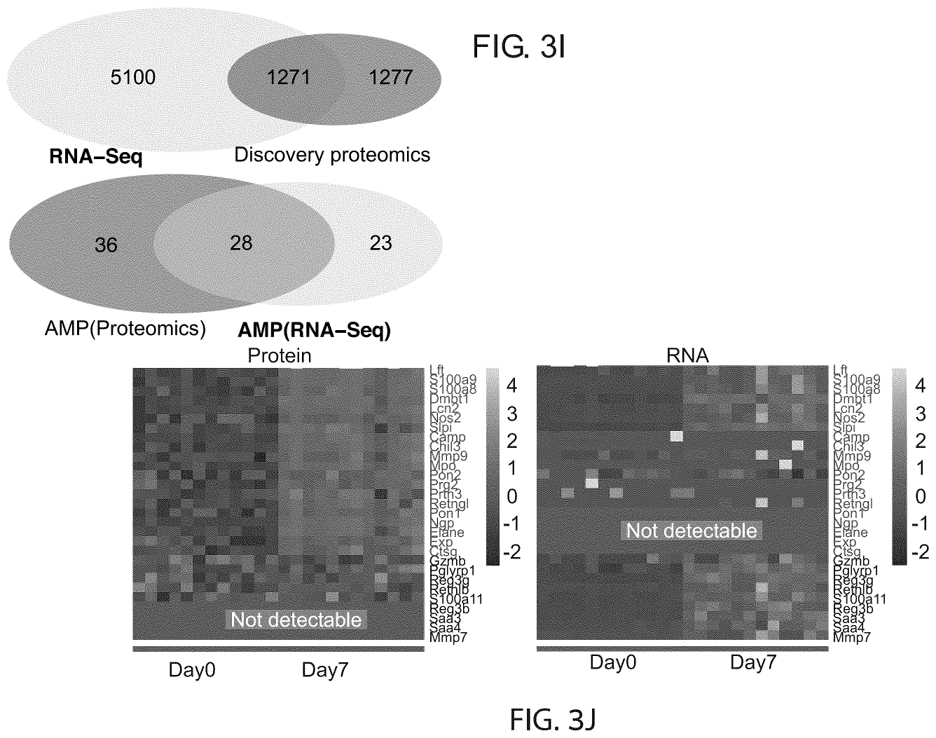

Whilst conceiving embodiments of the invention, the present inventors have now configured a novel approach for characterizing the innate immune response, and, in particular, antimicrobial peptides and proteins (AMPs). The approach is based on identification of AMPs according to functional as well as structural criteria. Based on this approach the present inventors explored the proteomic host AMP-microbiome interactome by systematically characterizing the landscape of known and candidate AMPs along the murine gastrointestinal (GI) tract and its impact on gut microbiome dynamics and disease phenotypes. By employing a mass spectrometry-based high-throughput AMP measurement pipeline integrating mucosal AMP analysis as a discovery approach and stool AMP profiling as a targeted and diagnostic proteomic tool, the present inventors have described a region-specific, microbiome-dependent AMP repertoire along the healthy murine gut (FIGS. 1A-1J) and have demonstrated that protein-level AMP quantification is superior to AMP quantification at the transcriptional level (FIGS. 3A-3J).

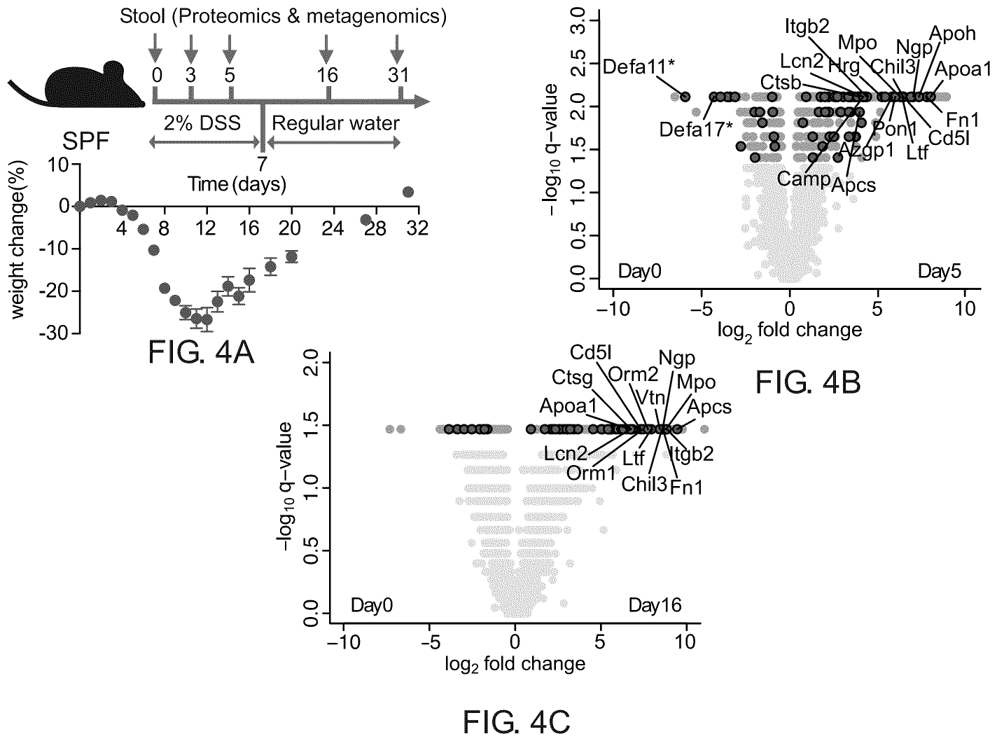

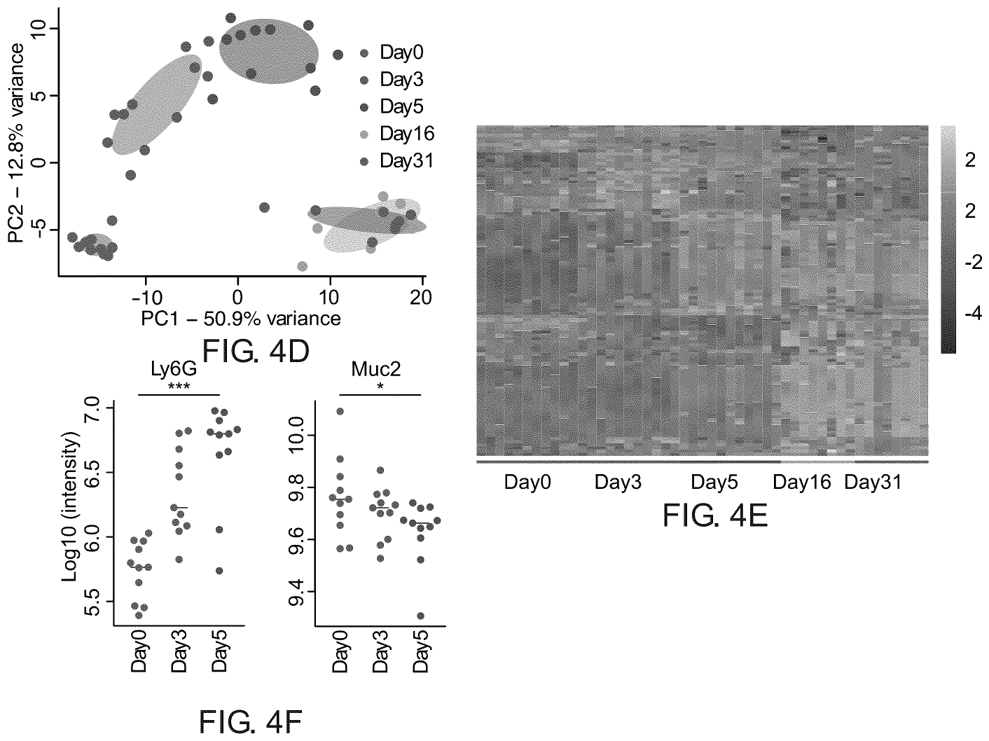

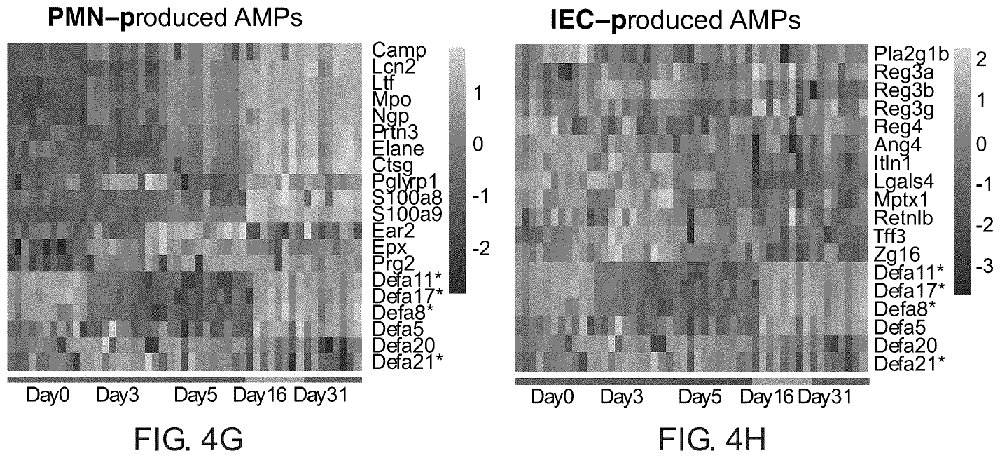

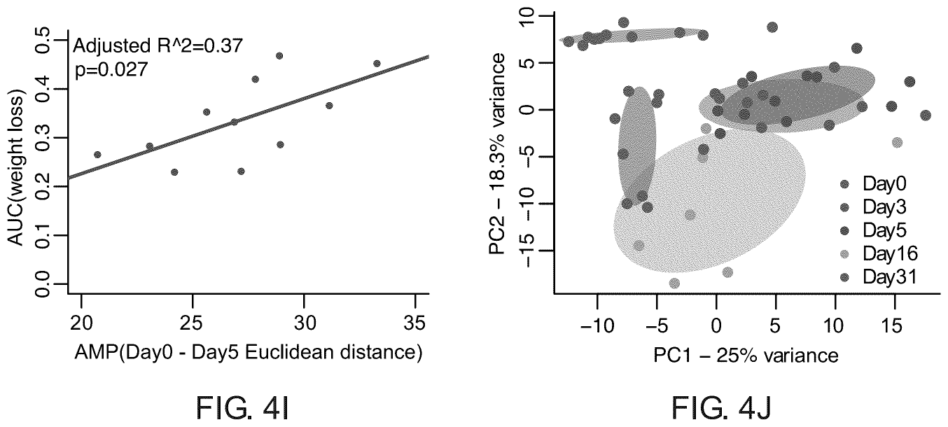

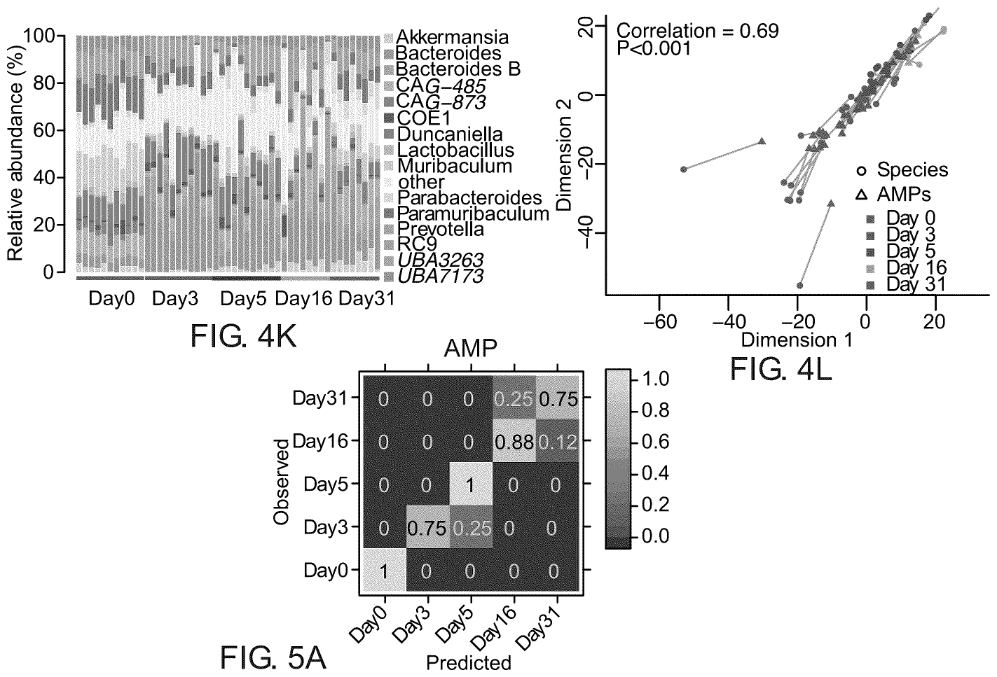

Using this approach, the present inventors characterized proteomic AMP hallmark responses upon commensal mono-colonization, enteric pathogenic infection, and intestinal inflammation (FIGS. 1E-1J, 2A-2N and 4A-4L). Furthermore, by integrating context-specific host proteome and microbial metagenome signatures, they explored the interplay between host AMPs and intestinal microbiome community structure in health and during disease-associated dysbiosis development and resolution (FIGS. 4A-4L, 5A-5G and 6A-6I).

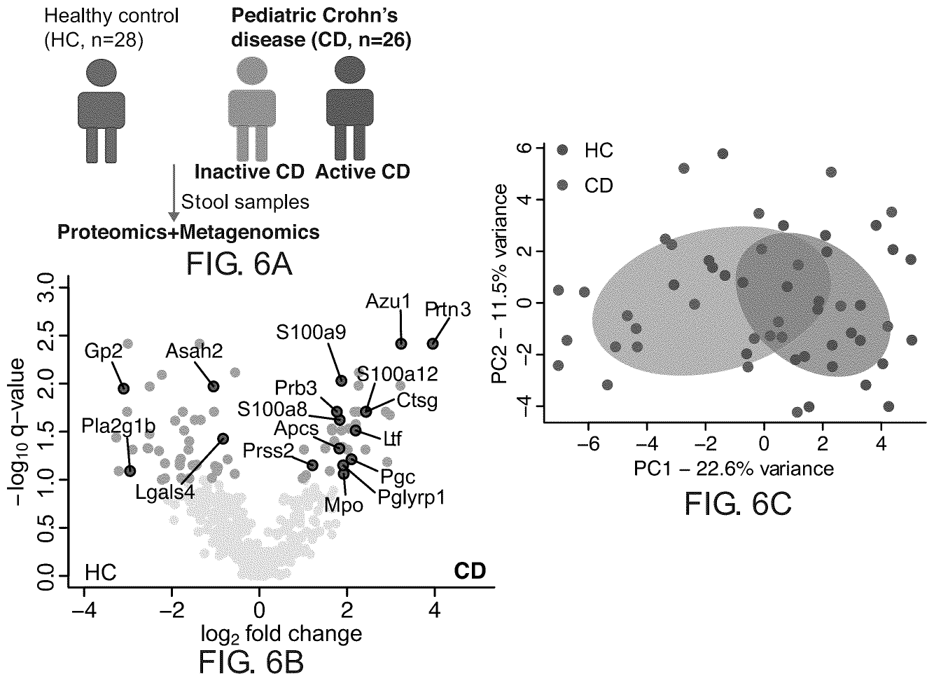

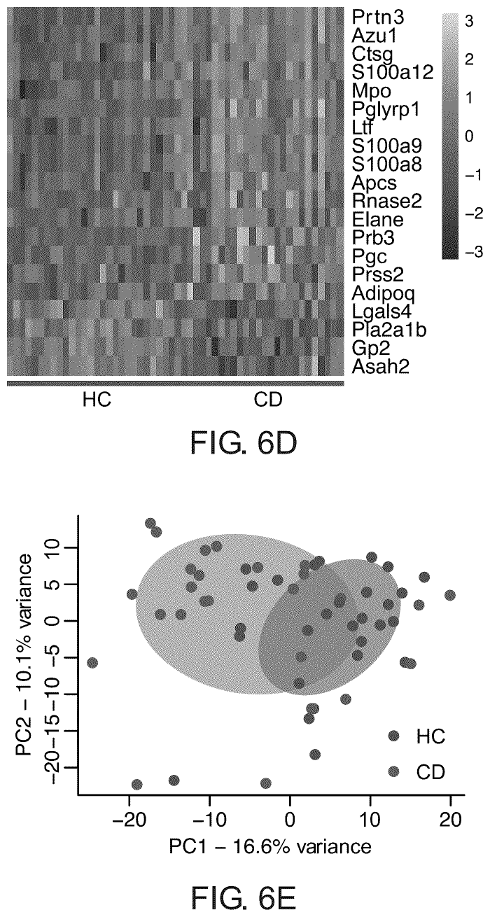

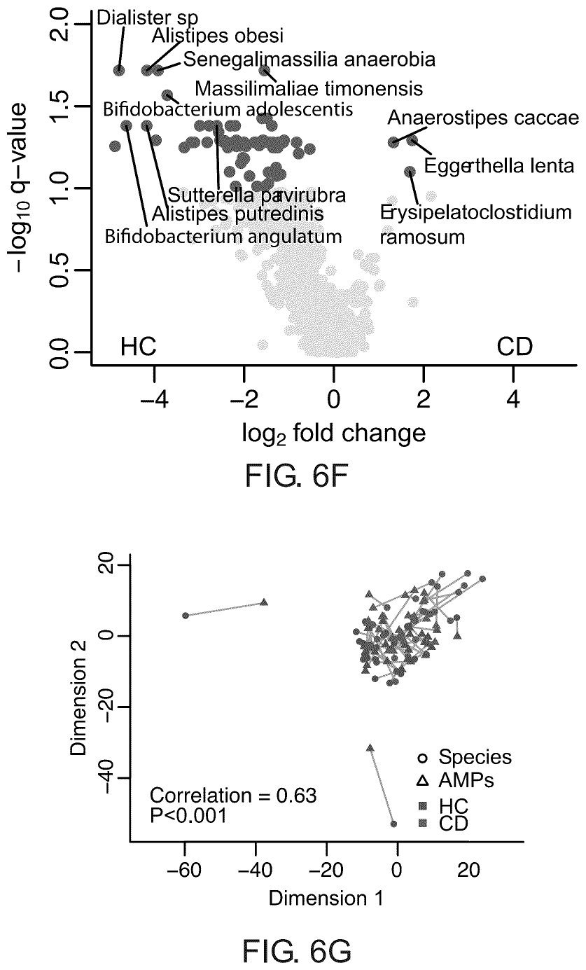

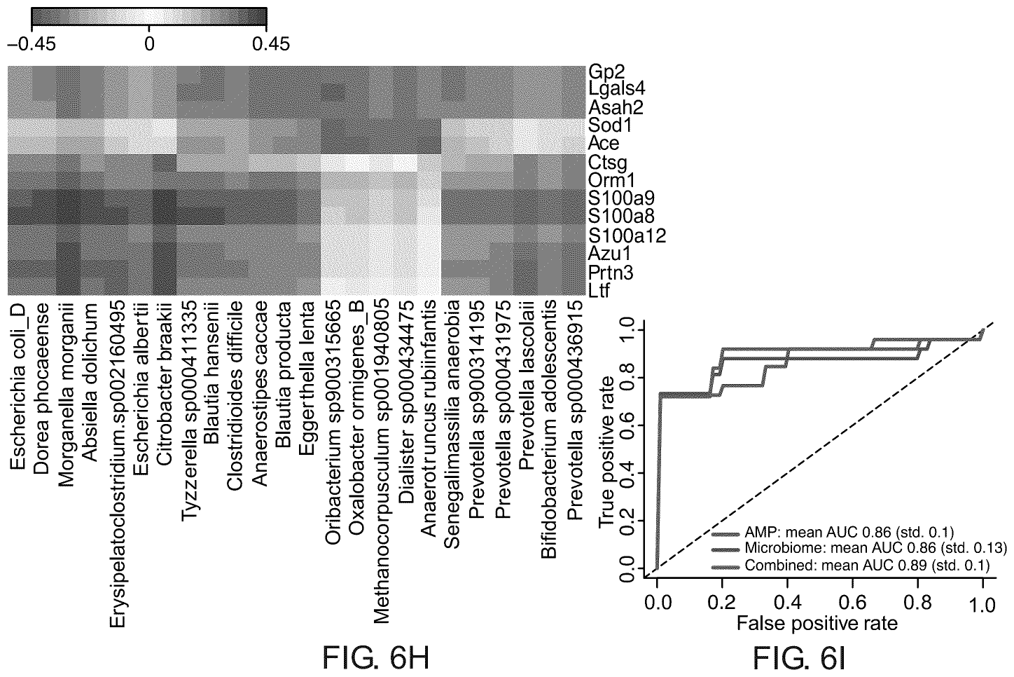

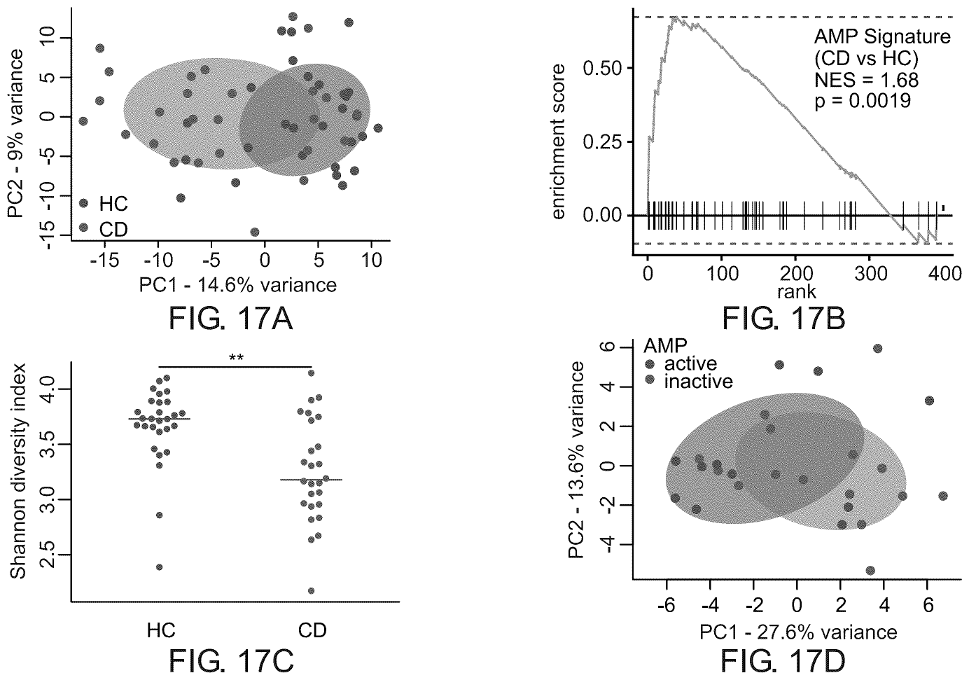

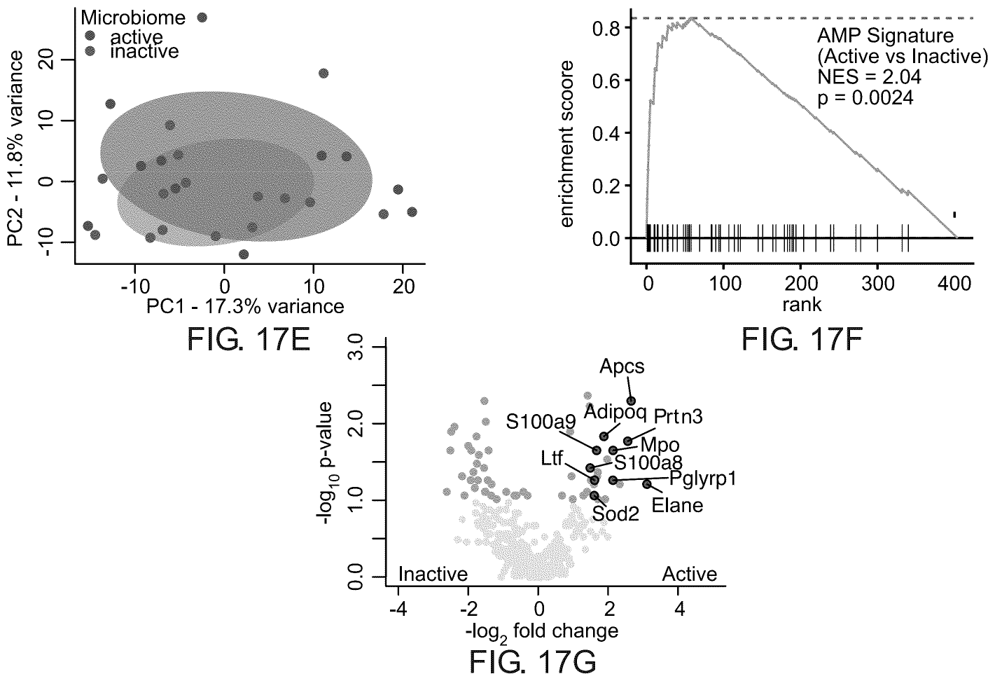

Importantly, by correlating niche-specific AMP signatures along the GI tract with the fecal AMP signature, the present inventors have shown that the fecal AMPs can serve as an accessible and faithful proxy for the proteomic AMP signature in the large intestine. Further, the present inventors have shown that the AMP landscape is predictive of the microbiome dynamics and intestinal inflammatory disease outcome, and demonstrated that application of AMP proxies in human IBD can faithfully differentiate between disease and healthy conditions and predict disease activity dynamics (FIGS. 6A-6I and 17A-17G), enabling diagnosis and monitoring of microbiome-associated disease.

Thus, according to one aspect of the present invention there is provided a method of determining the physiological state of a tissue of a subject, the method comprising:

measuring the levels of a plurality of antimicrobial peptides (AMPs) in a sample of a secretion, exudate or excretion of the tissue of the subject, wherein the plurality of AMPs is predominantly AMPs having shared abundance in the tissue and in the secretion, exudate or excretion of the tissue in a predetermined physiological state, and wherein a statistically significant correlation between the levels of the plurality of AMPs in the secretion, exudate or excretion and the levels of the plurality of AMPs in the tissue in the physiological state is identified,

determining the presence of the physiological state of the tissue, wherein levels of the plurality of AMPs constitutes a proxy for the physiological state of the tissue.

As used herein, the term “physiological state of a tissue” refers to clinical parameters characterizing a tissue, e.g. function, structure, molecular markers, chemical status, metabolic status, histological status, morphological status, electrochemical status. These clinical parameters characterize the physiological state, which can be healthy or diseased or inbetween e.g., at risk or prone to develop a disease. Physiological state may also refer to gene expression, different states of differentiation, such as an intermediate state, an immune state (e.g., dysfunctional, naive, memory state) and a disease state (e.g., infected, malignant state). Tissues can have different states based upon the composition of cells in a microenvironment or location of the tissue. In some embodiments, the physiological state of the tissue refers to at least one of the immunological state, metabolic state, physical state and chemical state of the tissue.

The methods of the present invention can determine the physiological state of a tissue according to well-known parameters. Exemplary parameters of the immunological state of a tissue include, but are not limited to the balance of pro-inflammatory cytokines (e.g. IL-1beta, TNFalpha, IL-12, IL-18, GCSF and IFNgamma) and anti-inflammatory cytokines (e.g. IL-10, IL-11, IL-1ra, IL-4, IL-13 and TGFbeta) in the tissue, measuring the presence of immune cells such as CD8+ Tcells, natural killer (NK) and natural killer T (NKT) cells, gammadelta T cells, antigen-presenting cells, alphabeta T cells, regulatory T (Treg) cells, beta-lymphocytes and phagocytes in the tissue.

Exemplary parameters reflecting the metabolic state of a tissue include, but are not limited to oxygen consumption, energy metabolism (e.g. carbohydrate and lipid metabolism), fluctuations in metabolic gene and protein expression, blood perfusion, electro-chemical activity and levels of enzymatic (e.g. glycolysis, Krebs cycle and enzymes) activity.

Exemplary parameters reflecting the physical state of a tissue include, but are not limited to thermal properties (e.g. heat conductivity, thermal conductivity, specific heat capacity, thermal resistance), optical properties (e.g. absorption coefficient and scattering coefficient, refractive index), mechanical properties (e.g. viscoelasticity, elasticity, stiffness, tensile and compressive strength, contractability [muscle]), acoustic properties (e.g. sound speed, attenuation, non-linearity), dielectric properties (e.g. permittivity, conductivity) and weight.

Exemplary parameters reflecting the chemical state of a tissue include, but are not limited to pH, dry weight and water content, biochemical composition (e.g. fat, lipid, carbohydrate, protein, nucleic acid, etc.) and chemical composition (elements and molecules) of the tissue.

As used herein, the term “tissue” refers to a group of structurally and functionally related cells typically of more than one type and their intracellular material. Biological tissues can be grouped according to cell type, as in epithelial, endothelial, stromal and connective tissues, or according to function, such as connective tissue; nervous, contractile (e.g. muscle) and endocrine tissue; alimentary (e.g. gastro-intestinal) tissue and pulmonary (e.g. lung) tissue. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, genitourinary tissue, ocular tissue, pulmonary tissue, gonadal tissue, hematopoietic tissue. In specific embodiments, the tissue is said tissue is selected from the group consisting of gut tissue, vaginal/cervical/uterine tissue, nasopharynx, lung tissue, genitourinary tissue, ocular tissue and skin.

In particular embodiments, the physiological state of gut tissue is determined. Gut tissue is typically comprised of a mucosal layer, submucosal layer, muscular layer and a serous layer, though embodiments of the invention can relate to parts thereof. The mucosa, which lines the lumen of the digestive tract, comprises epithelium, connective tissue and a smooth muscle layer. In specific embodiments, the methods determine physiological state of the gut mucosa.

In one step of some embodiments of the methods of the invention, the physiological state of a tissue of a subject is determined by measuring levels of a plurality of antimicrobial peptides in a sample of a secretion, exudate or excretion of the tissue. As used herein, the term “antimicrobial peptide” refers to oligo- or polypeptides that kill microorganisms or inhibit their growth. “Antimicrobial peptides” (AMPs) may include peptides that result from the cleavage of larger proteins or peptides that are synthesized ribosomally or non-ribosomally. Generally, antimicrobial peptides are cationic molecules with spatially separated hydrophobic and charged regions. Exemplary antimicrobial peptides include linear peptides that form an alpha-helical structure in membranes or peptides that form beta-sheet structures optionally stabilized with disulfide bridges in membranes. Representative antimicrobial peptides include, but are not limited to cathelicidins, defensins, dermcidin, and more specifically magainin 2, protegrin, tachyplesin, protegrin-1, melittin, dermaseptin 01, cecropin, caerin, ovispirin, alamethicin, pandinin 1, pandinin 2, and mastoparans B.

It will be appreciated that AMPs can be identified by the entire AMP protein or peptide, but will also refer to fragments of an AMP (i.e. a partial amino acid sequence of the AMP) which are indicative of the presence of the AMP. Such fragments may be the products of proteolytic digestion or chemical processes, for example, gut AMPs which are partially digested by endogenous proteases, such as trypsin, or partially digested by microbial proteases from commensal and/or pathological organisms comprising the gut microbiome. One example of such fragments, produced by trypsin digestion is the list of peptides representing AMPs identified in fecal samples in Table S2 below.

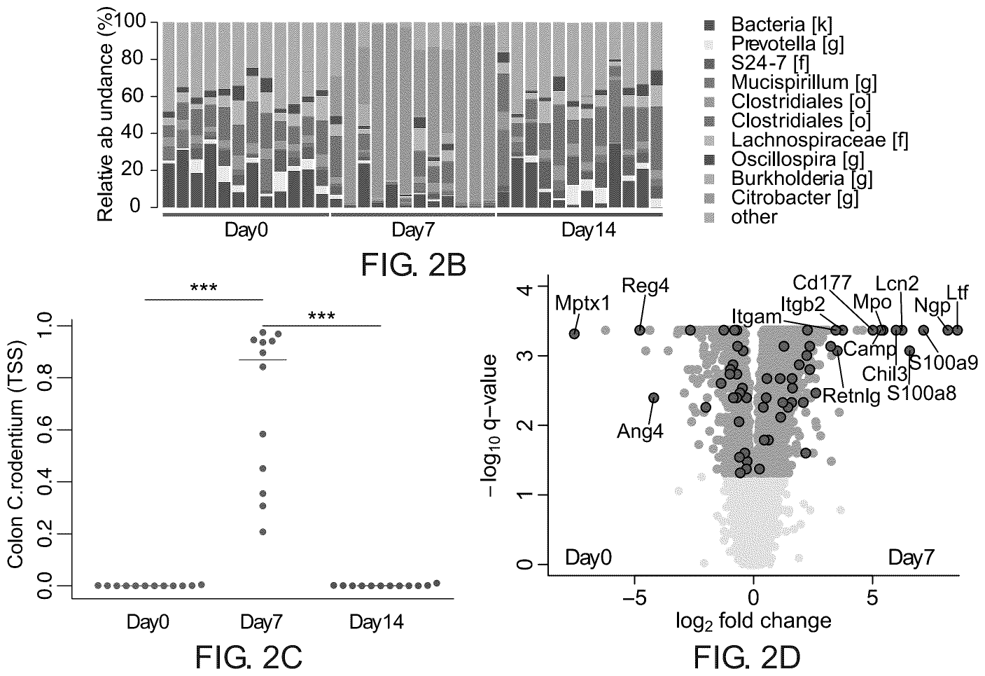

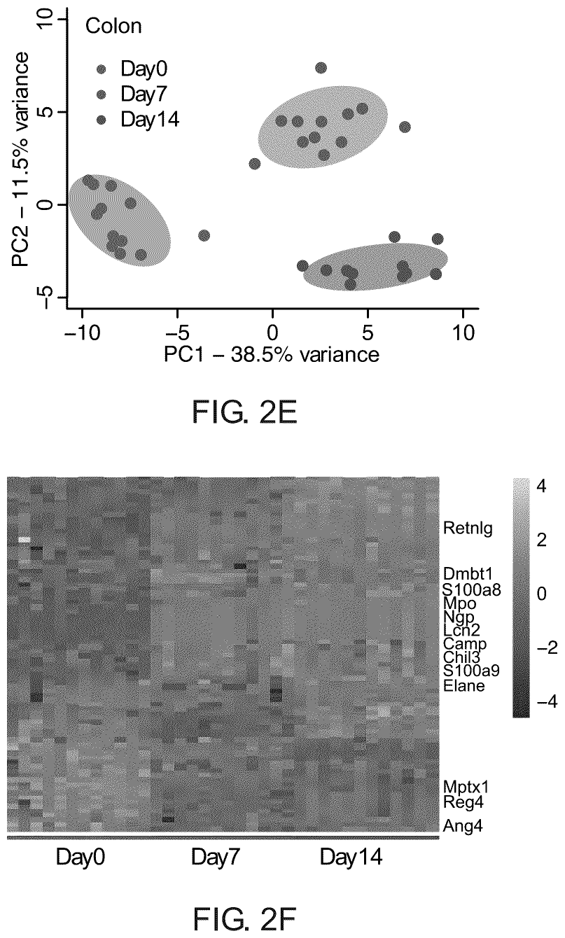

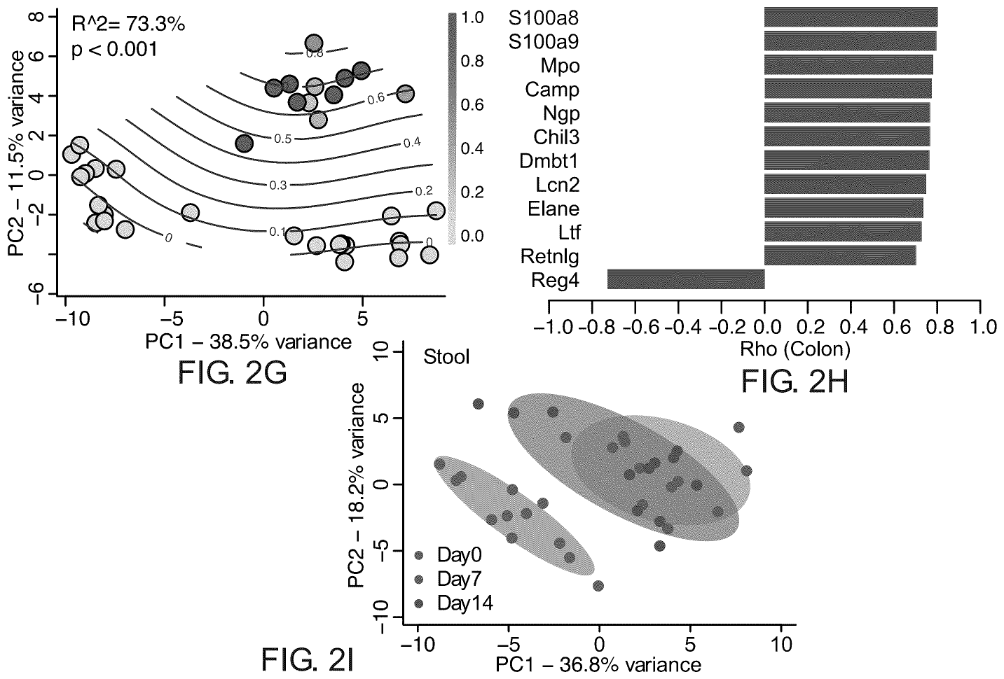

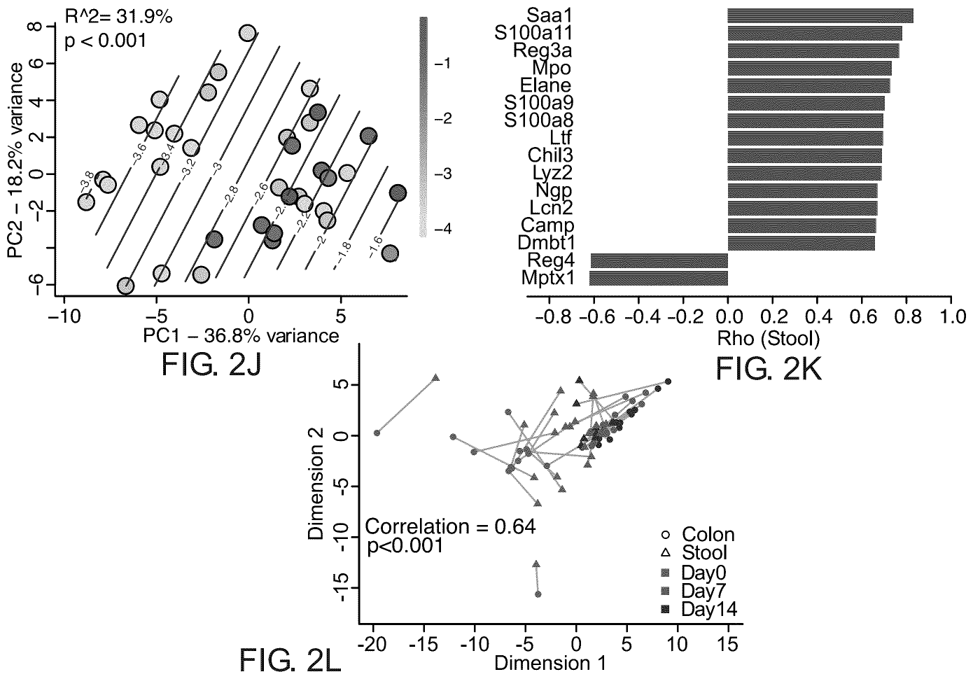

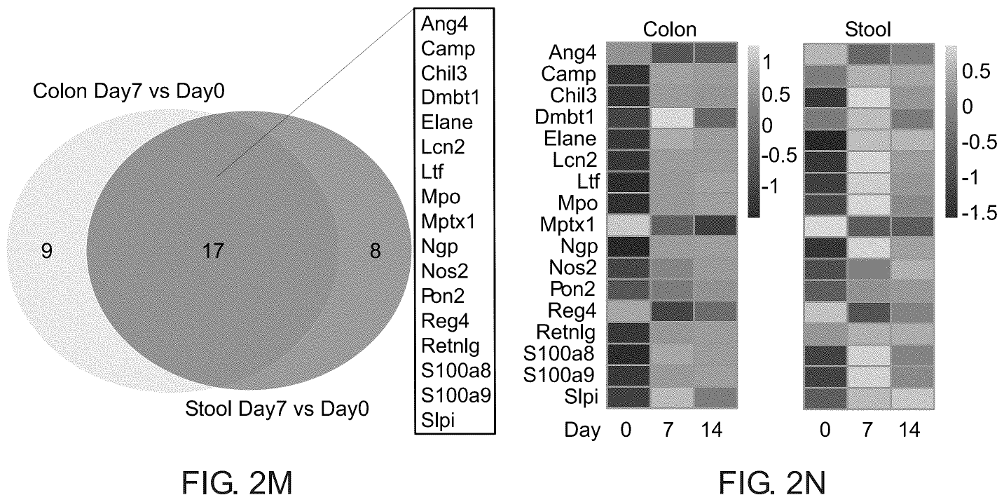

Using proteomic techniques, the inventors have established novel criteria for determining the AMP profile of a tissue or other biological sample, and have elucidated the AMP profile of the gut mucosa, at different locations and in response to changes in the gut microbiome (see, for example, FIGS. 1A-1J and 3A-3J hereinbelow), as well as the corresponding fecal AMP profiles (see, for example, FIGS. 2A-2M).

Thus, in some embodiments the method comprises measuring a plurality of AMPs, wherein the AMPs comprise:
a) annotated antimicrobial proteins and peptides, and
b) proteins and peptides having at least 2 out of the following criteria:
i) secreted protein or peptide;
ii) protein or peptide having two or more of amino acids arginine, lysine or histidine, having a net charge between −5 and +10, having hydrophobic content between 10% and 80% and less than 200 amino acids in length;
iii) protein or peptide comprising a bacterial ligand-binding domain;
iv) protein or peptide having an enhancing antibacterial defense response activity;
v) protein or peptide having immunomodulatory activity, and
vi) protein or peptide having a tissue distribution pattern similar to AMP disclosed in an antimicrobial protein and peptide database upon commensal colonization, pathogenic infection or intestinal inflammation.

Thus the AMPs may be known, annotated AMPs or proteins or peptides which are AMP “candidates”, having at least two of the properties (b)(i)-(vi). In some embodiments the AMP “candidates” have two, three, four or more of properties (b)(i)-(vi).

As used herein, annotated AMPs include, but are not limited to, AMPs identified in the published literature, e.g. databases. Annotated AMPs can be AMPs from at least one database, including but not limited to Uniprot, the Antimicrobial Peptide Database (APD), Data Repository of Antimicrobial Peptides (DRAMP 2.0), Database of Antimicrobial Activity and Structure of Peptides (DBAASP) and Collection of antimicrobial peptides (CAMP).

Additional AMPs identified from the proteome are candidate AMPs (peptides or proteins not identified as AMPs in published literature), having at least two of criteria (b)(i)-(vi).

Thus, in some embodiments the candidate AMP is a secreted peptide or protein. Proteins or peptides can be identified as secreted according to the published literature, as annotated as “secreted” in a protein or peptide database (e.g. Uniprot dot org), or predicted secreted using signal peptide prediction software (e.g. SignalP from the Center For Biologicals Sequence Analysis CBS).

In some embodiments, the candidate AMP is identified according to structural similarity to known AMPs. Structural similarity can include the presence of specific protein folds, domains or structural motifs similar to known AMPs, or as identified by structural prediction algorithms (e.g. pfam dot xfam), as well as high level of sequence identity or similarity to known AMPs, as identified by sequence local alignment algorithms (e.g. BLAST, CLUSTAL, and the like). In specific embodiments, the candidate AMPs have structural similarity when having two or more of amino acids arginine, lysine or histidine, having a net charge between −5 and +10, having hydrophobic content between 10% and 80% and less than 200 amino acids in length.

In some embodiments, the candidate AMP is identified by the presence of a bacterial ligand-binding domain. Bacterial ligand binding domains can be identified in the published literature, as annotated in the Uniprot database, or as identified as such by structure prediction algorithms (e.g. pfam dot xfam). In specific embodiments, the bacterial ligand binding domain includes motifs capable of binding bacterial lipopolysaccharades (LPS), bacterial lectins, bacterial lipoteichoic acids (LTA) and/or peptidoglycan precursor (Lipid II).

In some embodiments, the candidate AMP is identified by the presence of an enhanced antibacterial defense response activity. Antibacterial defense response activity can include, but is not limited to phagocytosis, bacterial membrane pore formation, radical oxygen species (ROS) production, limitation of free nutrient/inorganic ions and hydrolytic enzyme release. In some embodiments, antibacterial defense response activity can also include promotion of growth of competing bacterial species, which limit proliferation of (e.g. undesired) species.

Some AMPs exert their activity via immune-modulation. Thus, in some embodiments, the candidate AMP is identified by the presence of an immune-modulatory activity Immune-modulatory activity of candidate AMPs can include, but is not limited to chemoattraction of monocytes, neutrophils, dendritic cells, and T-cells, chemokine and cytokine production and neutrophil degranulation.

Candidate AMPs can also be identified by their differential abundance. In some embodiments, proteins or peptides the levels of which increase or decrease along with those of known (e.g. annotated) AMPs can be candidates for AMPs. For example, where levels of a known AMP or AMPs are elevated in a tissue in response to a pathogenic infection (e.g. C. difficil infection of the gut), commensal colonization or inflammation (e.g. IBD), a protein or peptide of interest can be considered a candidate AMP when, in addition to possessing at least one other of criteria (b)(i)-(v), it is also elevated in the same tissue upon the same infection or inflammation. In some embodiments, greater importance is attributed to the criterion of differential abundance when determining the identity of AMPs from candidate AMPs.

Example 1 of the Examples section details the identification of AMPs from the murine gut according to one embodiment of the invention. Proteomic analysis of samples from the ileal, cecal and colonic regions of the gut mucosa revealed 141 proteins and peptides, of which 116 were identified as annotated AMPs (Table S1), as well as 25 AMP candidates (Table S1A) having at least two of the criteria of (b)(i)-(vi), comprising an AMP profile of the gut. Thus, in some embodiments, the AMPs are gut AMPs selected from the group consisting of Angiotensin-converting enzyme 2, Adiponectin, Neutral ceramidase, Zinc-alpha-2-glycoprotein, CD177 antigen, Chitinase-like protein 3, Tetranectin, Clusterin, Cystatin-A; Cystatin-A, N-terminally processed, Cystatin-B, Cathepsin D, Cathepsin E, Cathepsin L1; Cathepsin L1 heavy chain; Cathepsin L1 light chain, Hyaluronan-binding protein 2, Integrin alpha-M, Integrin beta-2, Mesencephalic astrocyte-derived neurotrophic factor, Matrix metalloproteinase-9, Mucosal pentraxin, Cytosolic phospholipase A2 gamma, Group XV phospholipase A2, Lithostathine-1, Lithostathine-2, Resistin-like gamma and Protein S100-A13.

In particular embodiments, the AMPs are gut AMPs having amino acid sequences selected from the group consisting of SEQ ID NO: 3-27. In other embodiments, the AMPs are gut AMPs having amino acid sequences at least 75, 80, 85, 90, 95, 96, 97, 98, or 99% identical to a sequence selected from SEQ ID NO: 3-27.

In particular embodiments, the tissue is human gut tissue and the gut AMPs are human orthologs of murine AMPs identified by the methods of the invention.

In some embodiments, the tissue is human gut tissue and the gut AMPs are human orthologs of amino acid sequences selected from the group consisting of SEQ ID NO: 3-27.

While determining the AMP profile of the gut, and the AMP profile of corresponding stool samples from the same mice, the present inventors revealed a number of AMPs which were present in both the gut and stool samples, and whose abundance fluctuated (increased or decreased) in significant correlation between the two sample types, demonstrating that the patterns of change of the gut AMPs in the course of pathogenic bacterial infection are reflected in those of a number of the fecal AMPs analyzed in the same manner (see FIGS. 2K-2N).

Thus, in one aspect of some embodiments of the invention, one step of the method comprises measuring the levels of a plurality of antimicrobial proteins or peptides (AMPs) in a sample of a secretion, exudate or excretion of the tissue of the subject, wherein the plurality of AMPs is predominantly AMPs having shared abundance in the tissue and in the secretion, exudate or excretion of the tissue in a predetermined physiological state. In some embodiments, the tissue is gut tissue, the excretion is feces (stool), and the AMPs having shared abundance in the gut and in the feces are selected from the group consisting of the AMPs listed in Table S2 hereinbelow. In particular embodiments, the tissue is human gut tissue, and the AMPs having shared abundance in the gut and feces are human orthologs of murine AMPs selected from the group consisting of the AMPs listed in Table S2.

As used herein, the term “plurality of AMPs” refers to a group of AMPs, numbering more than 1. In some embodiments, the plurality of AMPs comprises 2, 3, 4, 5, 6, 7, 8, 9, 10, 11, 12, 13, 14, 15, 16, 17, 18, 19, 20, 25, 27, 30, 32, 35, 37, 40, 45, 50, 55, 60, 65, 70, 75, 80, 85, 90, 95 or 100 AMPs. In specific embodiments, the plurality of AMPs comprises greater than 5 and fewer than 50 AMPs, greater than 10 and fewer than 100 AMPs.

As used herein, the terms “secretion”, “exudate” or “excretion” refer to waste products, fluids or solids produced and released by the tissue, fluids or solids which have come in contact with the tissue, and can be accessed without sampling the tissue. Exemplary secretions and their related tissues include, but are not limited to perspiration and the skin, milk and the breast/mammary gland, gastric fluid and the stomach mucosa, and intestinal mucus and the gut mucosa. Exudates are fluids that filter from the circulatory system to lesions or areas of inflammation, usually the result of changes in vascular permeability. Exemplary exudates and their related tissues include, but are not limited to pus and infected tissue, and catarrhal exudate and the lining of the nasopharynx.

All living tissues excrete waste products of their metabolism. Also available for sampling are the waste products of digestion. Exemplary excretions (excreta, excrement) and their related tissues include, but are not limited to feces and the large intestine/colon, urine and the urinary tract, and mucus and the airways.

As used herein, the phrase “shared abundance” refers to a statistically significant correlation between the fluctuations or perturbations (increase, decrease) in levels of the AMP or AMPs in the tissue and in the “secretion”, “exudate” or “excretion” thereof. In some embodiments, where a specific AMP thereof reveals the same trends in abundance in both the tissue and in the secretion, exudate or excretion thereof, is classified as having “shared abundance”. In other embodiments, the statistical correlation of the trends is determined according to statistical tests including, but not limited to Mann-Whitney U test (for independent comparison), Wilcoxon signed-rank test (for paired comparison) with FDR correction and calculation of Log 2 fold changes. Further, non-random behavior of the proteomic AMP signature against the background of the entire proteomic landscape may be analyzed by applying pre-ranked protein set enrichment analysis using the “fast gene set enrichment analysis” method (Korotkevich et al., 2019), with ranks calculated as signed fold change ′−log 10 p-value. Intersecting protein signatures can also be identified using Venn diagrams. For proteomic comparison of more than one group, in some embodiments, the Kruskal—Wallis one-way analysis of variance (for independent comparison) or one-way repeated measures ANOVA on rank transformed values (for paired comparison) can be used.

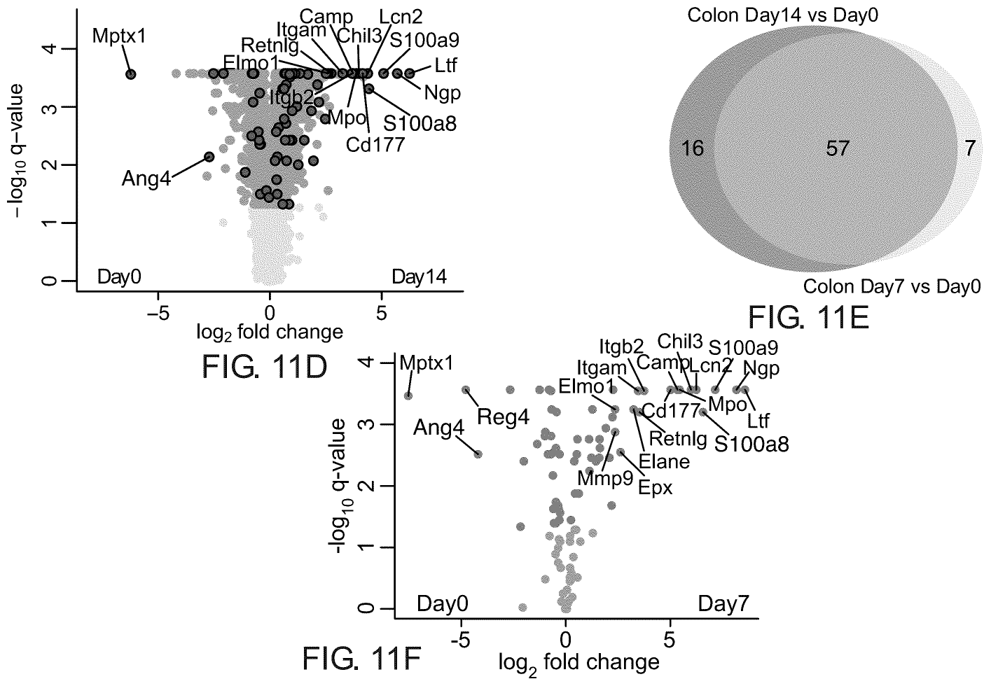

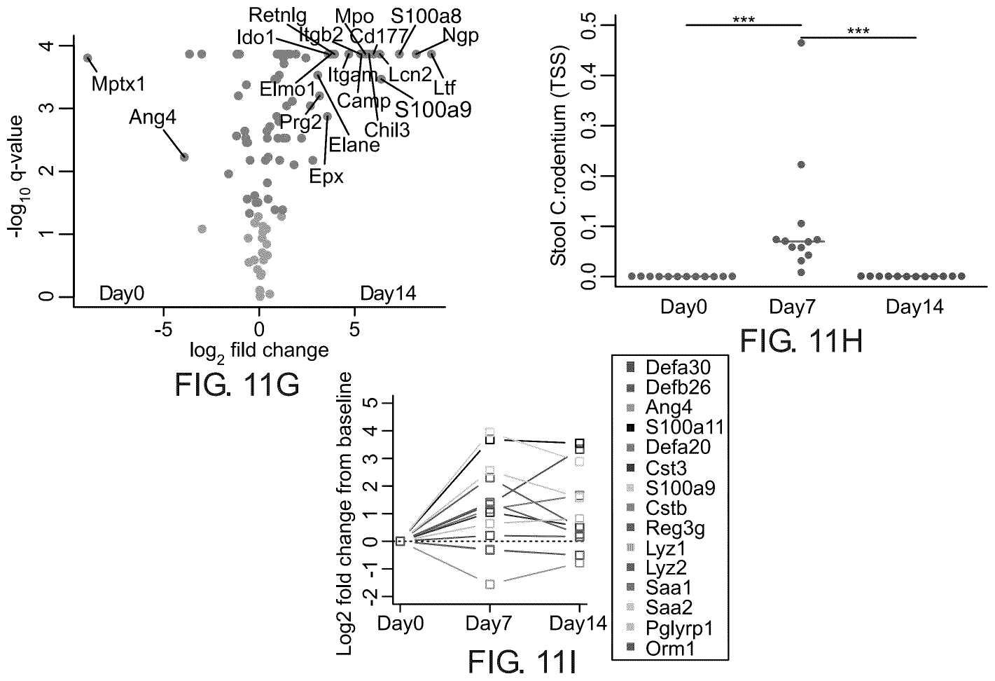

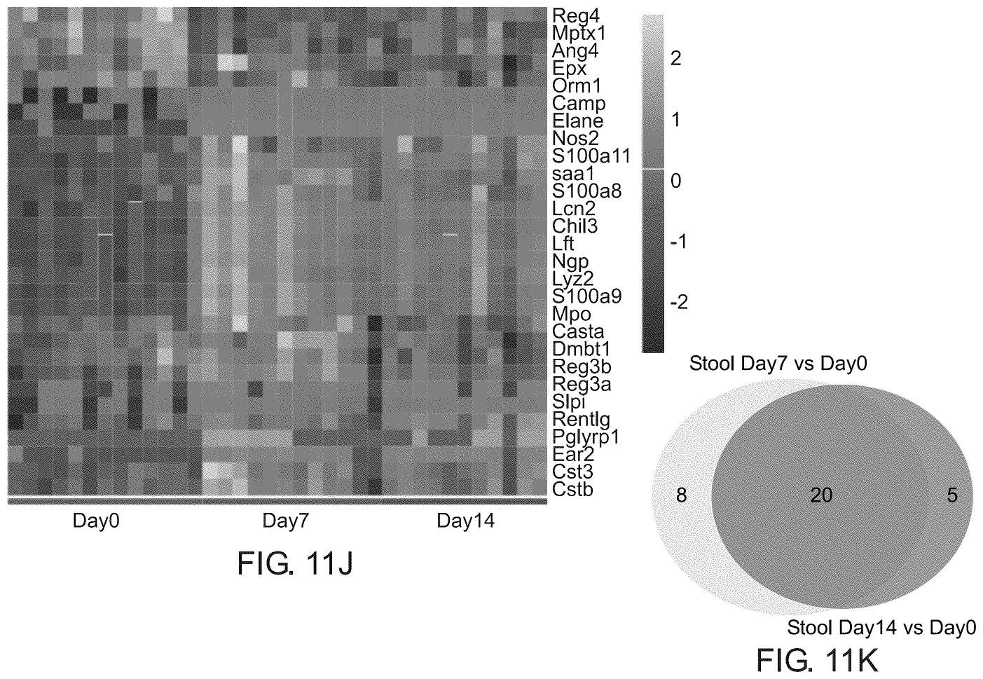

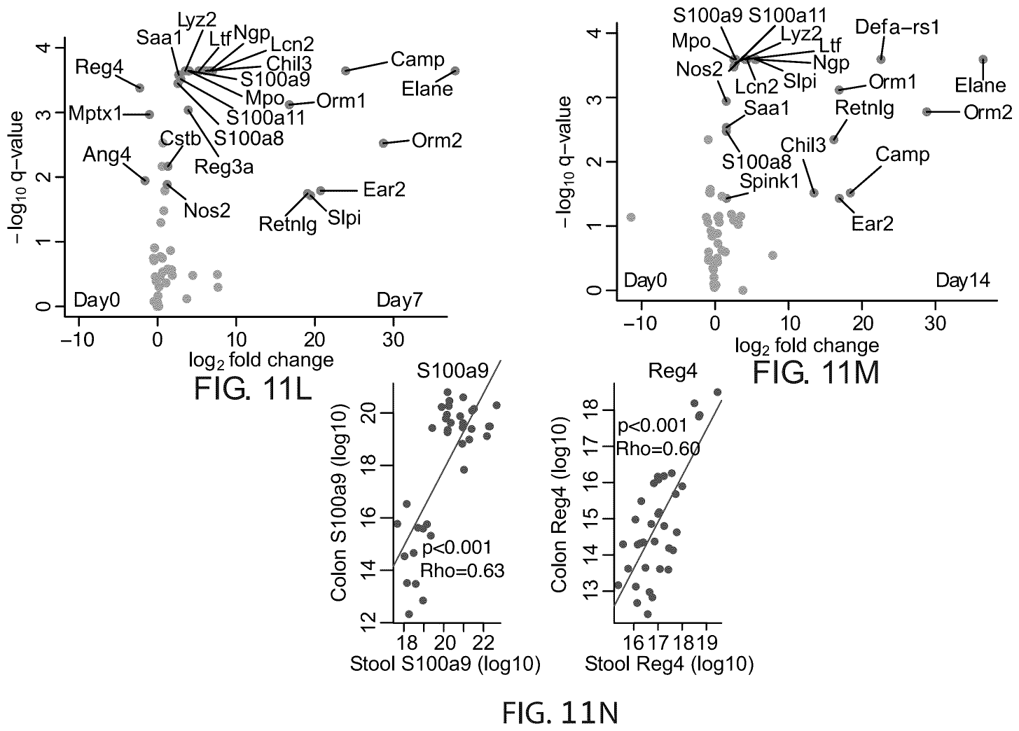

In an exemplary embodiment, fecal (stool) AMPs have a shared abundance with gut AMPs when their levels increase or decrease with a statistically significant correlation in response to colonization by a commensal bacteria, or in response to infection by a pathogenic microorganism (e.g. C difficil), or in response to disease (inflammation, e.g. IBD). “Shared abundance” may also refer to a consistent parallel course of fluctuation of the AMP(s) over the duration of a condition, for example, commensurate increases or decreases in the levels of gut AMPs and fecal (stool) AMPs at the onset, peak and recovery phases of a pathogenic gut infection (see, for example FIG. 11C and FIG. 11H, FIGS. 2I, 2M and 2N). In some embodiments, fecal (stool) AMPs having shared abundance with gut AMPs in response to a pathogenic bacterial infection are one or more AMPs selected from the group consisting of angiogenein 4 (Ang4), cathelicidin antimicrobial protein (Camp), chitinase-like protein 3 (Chil3), deleted in malignant brain tumors 1 protein (Dmbt1), neutrophil elastase (Elane), neutrophilic gelatinase-associated lipocalin (Lcn2), lactotransferrin (Ltf), myeloperoxidase (Mpo), mucosalntraxin (Mptx 1), neutrophilic granular protein (Ngp), nitric oxide synthase, inducible 2 (Nos2), serum paraoxonase/arylesterase 2(Pon2), regenerating islet derived protein 4 (Reg4), Retnil, S100a8, S100a9 and antileukoproteinase (Slpi). In particular embodiments, the tissue is human tissue, and the one or more AMPs are selected from the group consisting of human orthologs of murine angiogenein 4 (Ang4), cathelicidin antimicrobial protein (Camp), chitinase-like protein 3 (Chil3), deleted in malignant brain tumors 1 protein (Dmbt1), neutrophil elastase (Elane), neutrophilic gelatinase-associated lipocalin (Lcn2), lactotransferrin (Ltf), myeloperoxidase (Mpo), mucosalntraxin (Mptx 1), neutrophilic granular protein (Ngp), nitric oxide synthase, inducible 2 (Nos2), serum paraoxonase/arylesterase 2(Pon2), regenerating islet derived protein 4 (Reg4), Retnil, S100a8, S100a9 and antileukoproteinase (Slpi).

Thus, in embodiments of the method of the invention, the plurality of AMPs is predominantly AMPs having shared abundance in the tissue and in the secretion, exudate or excretion of the tissue in a predetermined physiological state. As used herein, the term “in a predetermined physiological state” refers to one or more physiological states of the tissue for which there are AMP(s) from the secretion, exudate or excretion of the tissue having a known shared abundance in the tissue.

As used herein, the term “predetermined physiological state” refers to one or more (e.g. plurality) of physiological state or states for which AMPs having shared abundance in the tissue and in the secretion, exudate or excretion of the tissue have been identified, and for which levels measured in a sample from the secretion, exudate or excretion of the tissue can provide information regarding the physiological state of the tissue. In some embodiments, the predetermined physiological state refers to at least one of the immunological state, metabolic state, physical state and chemical state of the tissue.

Thus, in some embodiments, measuring the levels of the plurality of AMPs having shared abundance in a sample of a secretion, exudate or excretion of a tissue and in the tissue itself can determine the presence (or absence) of the predetermined physiological state of the tissue, without need for assaying the AMP levels of the tissue itself.

Thus, the levels of the plurality of the AMPs constitute a proxy for the physiological state of the tissue. As used herein, the term “proxy” refers to a “substitute” or a parameter or a set of parameters that is not a direct assessment of the physiological state, but is associated with it and can be used in place of the direct measure, albeit with acceptance of a possible greater degree of error in a resulting determination of the physiological state.

Thus, according to an aspect of some embodiments of the invention there is provided a method of determining a proxy for a physiological state or states of a tissue, the method the method comprising:
(a) determining an antimicrobial protein and peptide (AMP) profile of a sample of the tissue,
(b) determining the AMP profile of a sample of an exudate, secretion or excretion of the tissue and
(c) identifying a plurality of AMPs having shared abundance in the tissue and the exudate, secretion or excretion in a given physiological state, wherein the plurality of AMPs having shared abundance constitutes the proxy.

Methods for identifying AMPs having a shared abundance in the tissue and in the sample of an exudate, secretion or excretion of the tissue can include establishing an AMP profile for both the tissue and the sample of an exudate, secretion or excretion of the tissue, and identifying AMPs having commensurate differential and temporal fluctuations in both the tissue and the sample of an exudate, secretion or excretion of the tissue.

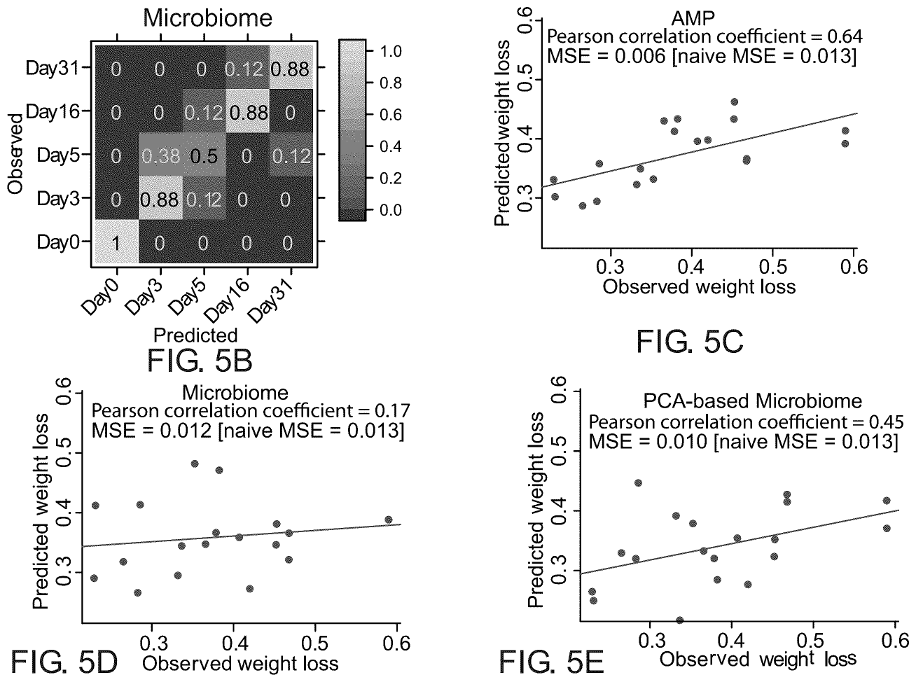

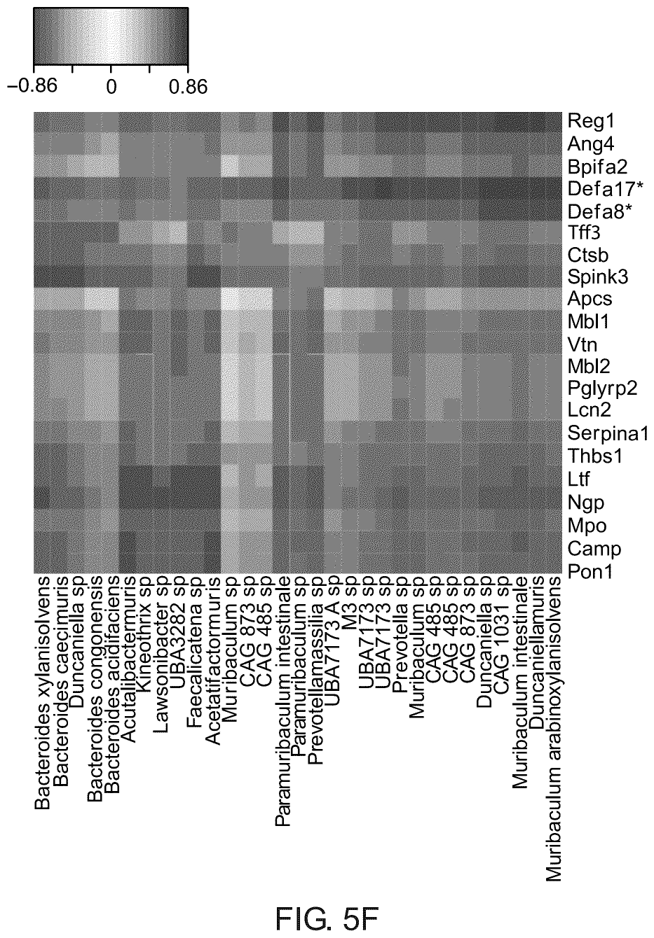

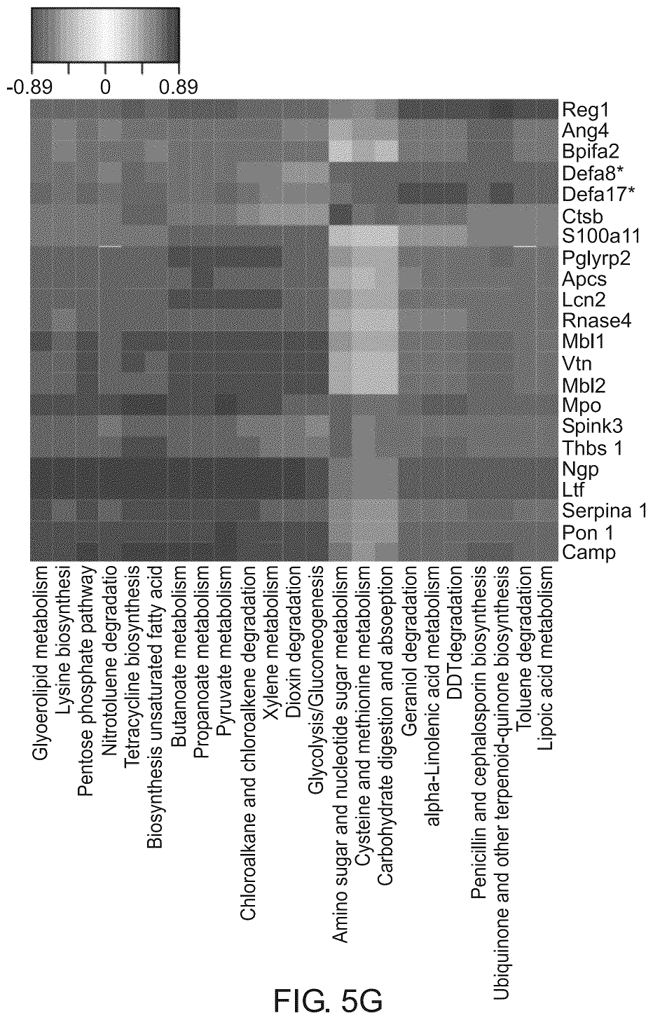

The present inventors have also shown that the profiles of annotated and candidate AMPs identified according to the methods of the invention in fecal (stool) samples from subjects with chronic intestinal inflammatory disease reflect the differential dynamics patterns characteristic of the extensive and complex alteration of the gut AMP profile and microbiome signature throughout the active disease as well as recuperation (see FIG. 5G). Specifically, the decreased abundance of Reg1, BPI fold-containing family A member 2 (Bpifa2), and IEC-produced AMPs (Ang4, alpha-defensins), together with increased abundance of PMN-produced AMPs (Lcn2, Pglyrp2, Ltf, Ngp, Mpo, Camp) and other AMPs (Apcs, Pon1, Mb1, Ctsb, Vtn), was predictive of the loss of commensal bacterial species and the blooming of colitis-associated species (FIG. 5F).

Thus, in some embodiments, there is provided a method of diagnosing and/or monitoring a disease of a tissue of a subject, the method comprising measuring the levels of a plurality of antimicrobial proteins or peptides (AMPs) in a sample of a secretion, exudate or excretion of said tissue of the subject, wherein the plurality of AMPs is predominantly AMPs having a statistically significant correlation with the changes in microbiome of the tissue and clinical parameters of the subject in a predetermined disease state and wherein a statistically significant correlation between said levels of said plurality of AMPs in said secretion, exudate or excretion and said levels of said plurality of AMPs in with the changes in microbiome of the tissue and clinical parameters of the subject in said predetermined disease state of said tissue is identified, determining the presence and/or severity or stage of said disease state of said tissue, and wherein levels of said plurality of AMPs constitutes a proxy for the disease state of the tissue.

The present inventors have revealed that both the gut and the fecal AMP profiles of subjects with pronounced gut inflammatory disease reflect the increase in neutrophil infiltration and intestinal epithelial damage, observed as an increase in PMN-produced AMPs (e.g. Camp, Lnc2, Ltf, Mpo, Ngp, Prtn 3, Elane, cathepsin G (Ctsg), Pglyrp1, S100a8, S100a9, and eosinophil AMPs Ear2, Epx and Prg2) and reduction in intestinal epithelial-produced AMPs (e.g. Ang4, Itln 1, Lgals4, Mptx 1, Retnlb and Zg16). Intestine epithelial AMPs Pla2g1b (phospholipase A2) and Reg proteins (Reg3a, Reg3g and Reg4) were also reduced at the onset of gut inflammatory disease but recovered in later disease phases. It will be appreciated that when the tissue is human tissue, and the samples are human samples, the AMPs indicative of the physiological state of the tissue are human AMPs, and, in particular, human orthologs of the indicated murine AMPs.

Thus, in some embodiments, the predetermined disease state is inflammatory disease of the gut, and the plurality of AMPs having a statistically significant correlation with the changes in microbiome of the tissue and clinical parameters of the subject in a predetermined disease state comprises one or more of Cathelicidin antimicrobial peptide (Camp), Lipocalin 2 (Lnc2), Lactotransferrin (Ltf), Myeloperoxidase (Mpo), Neutrophilic granule protein (Ngp), proteinase 3 (Prtn 3), neutrophil elastase (Elane), cathepsin G (Ctsg), Peptidoglycan recognition protein 1 (Pglyrp1), Protein S100A8 (S100a8), Protein S100A9 (S100a9), eosinophil cationic protein 2 (Ear2), eosinophil peroxidase (Epx), Bone Marrow Proteoglycan (Prg2), Angiogenin-4 (Ang4), Intelectin 1a (Itln 1), Galectin 4 (Lgals4), Mucosalntraxin (Mptx 1), Resistin-like beta (Retnlb) and zymogen granule membrane protein 16 (Zg16).

Employing such a plurality of AMPs, detection in a fecal (stool) sample of increased levels of one or more of Camp, Lnc2, Ltf, Mpo, Ngp, Prtn 3, Elane, Ctsg, Pglyrp1, S100a8, S100a9, Ear2, Epx and Prg2, and concomitant reduced levels of one or more of Ang4, Itln 1, Lgals4, Mptx 1, Retnlb and Zg16 suggests the presence of an inflammatory disease of the gut tissue. Likewise, reduced levels of Pla2g 1b and Reg3a, Reg3g and Reg4 and increased levels of one or more of Camp, Lnc2, Ltf, Mpo, Ngp, Prtn 3, Elane, Ctsg, Pglyrp1, S100a8, S100a9, Ear2, Epx and Prg2 can indicate early gut inflammatory disease, while reversion to normal levels of Pla2g 1b and Reg3a, Reg3g and Reg4 in a fecal (stool) sample can indicate progression towards recuperation from the gut inflammatory disease.

It will be appreciated that when the tissue is human tissue, and the fecal (stool) samples are human samples, the AMPs having shared abundance indicative of the physiological state of the tissue are human AMPs, and, in particular, human orthologs of the indicated murine AMPs.

In some embodiments, the AMP profiles can be established by first employing “label-free quantitative proteomics” (protease digestion, mass spectrometry and identification of the peptides by database searching) to identify the peptide “landscape” of the digested samples of the tissue and of the sample of an exudate, secretion or excretion of the tissue, as described herein, followed by identification of AMPs, including known, annotated AMPs as well as candidate AMPs, fulfilling the criteria described herein.

In some embodiments, the AMPs are identified by liquid chromatography techniques, mass spectrometry techniques, or by a combination of liquid chromatography and mass spectrometry techniques (LC-MS). Mass spectrometry techniques suitable for use with the methods of the invention include, but are not limited to Matrix-assisted laser desorption ionization-time-of-flight mass spectrometry (MALDI-TOF), Triple quadrupole mass spectrometry (TQMS or QqQ), Quadropole-Trap mass spectrometry (Q3), Hybrid linear ion trap (Orbitrap) mass spectrometry and Quadropole-Orbitrap mass spectrometry. Liquid chromatography techniques suitable for use with the methods of the invention include, but are not limited to reverse-phase (RP), ion exchange (including anion- or cation exchange, strong anion- or strong cation exchange), size exclusion, hydrophilic- and hydrophobic interaction, affinity chromatography and high performance liquid chromatography (HPLC).

In some embodiments, identification of AMPs from the tissue or from the exudate, secretion or excretion of the tissue is accomplished using AMP-binding moieties. As used herein, AMP binding moieties refers to any agent capable of specifically binding to an AMP, thus providing a means for isolating and identifying the AMP from a mixture of peptides or proteins. In some embodiments, the binding moieties can include, but are not limited to antibodies, antibody fragments, aptamers and the like.

The term “antibody” as used in this invention includes intact molecules as well as functional fragments thereof (that are capable of binding to an epitope of an antigen).

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

According to a specific embodiment, the antibody fragments include, but are not limited to, single chain, Fab, Fab′ and F(ab′)₂ fragments, Fd, Fcab, Fv, dsFv, scFvs, diabodies, minibodies, nanobodies, Fab expression library or single domain molecules such as VH and VL that are capable of binding to an epitope of the antigen in an HLA restricted manner.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv Fv (scFv), a disulfide-stabilized Fv (dsFv), an Fab, an Fab′, and an F(ab′)2, or antibody fragments comprising the Fc region of an antibody.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of immunological Interest, 5th ed., Public Health Service, NIH, Washington D.C.), location of the structural loop regions as defined by Chothia et al. (see, e.g., Chothia et al., Nature 342:877-883, 1989.), a compromise between Kabat and Chothia using Oxford Molecular's AbM antibody modeling software (now Accelrys®, see, Martin et al., 1989, Proc. Natl Acad Sci USA. 86:9268; and world wide web site www(dot)bioinf-org(dot)uk/dabs), available complex crystal structures as defined by the contact definition (see MacCallum et al., J. Mol, Biol. 262:732-745, 1996) and the “conformational definition” (see, e.g., Makabe et al., Journal of Biological Chemistry, 283:1156-1166, 2008).

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:
(i) Fv, defined as a genetically engineered fragment consisting of the variable region of the light chain (VL) and the variable region of the heavy chain (VH) expressed as two chains;
(ii) single chain Fv (“scFv”), a genetically engineered single chain molecule including the variable region of the light chain and the variable region of the heavy chain, linked by a suitable polypeptide linker as a genetically fused single chain molecule.
(iii) disulfide-stabilized Fv (“dsFv”), a genetically engineered antibody including the variable region of the light chain and the variable region of the heavy chain, linked by a genetically engineered disulfide bond.
(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;
(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);
(vi) F(ab′) 2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds);
(vii) Single domain antibodies or nanobodies are composed of a single VH or VL domains which exhibit sufficient affinity to the antigen; and
(viii) Fcab, a fragment of an antibody molecule containing the Fc portion of an antibody developed as an antigen-binding domain by introducing antigen-binding ability into the Fc region of the antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

As used herein, “aptamer” refers to short oligonucleotide or peptide molecules that bind to a specific target molecule (e.g. AMP). Aptamers typically include nucleic acid (DNA, RNA, XNA, split) and peptide aptamers. Similar to peptide aptamers are the larger protein “affimers”.

The AMP binding moieties can be used to identify AMPs in a liquid or solid phase. In some embodiments, the AMP binding moieties are immobilized on a substrate (e.g. chromatographic column, solid state array) for identification as well as isolation and purification of the AMPs. Thus, in some embodiments, the AMP binding moieties are immobilized on an array.

Identification of AMPs can be accomplished using the “forward” or “reverse” array technique-in the forward array, the AMP binding moiety is immobilized to the solid phase, and the AMP is identified (and/or isolated) following capture on the array. In the reverse array, the protein extract (comprising AMPs, e.g. of the tissue or the exudate, secretion or excretion of the tissue) is immobilized on the solid phase, and then contacted (e.g. incubated) with the AMP binding moiety (which can include a reporter moiety) for identification and isolation.

In some embodiments, such an AMP-binding moiety array can comprise greater than 10 and fewer than 100, between 5 and 100, between 10 and 100, between 5 and 50, about 10, about 20, about 30, about 40, about 50, about 60, about 70, about 80, about 90 or about 100 AMP binding moieties. In some embodiments the AMP binding moiety array comprises AMP binding moieties binding to the entire complement of AMPs comprising the plurality of AMPs constituting the proxy of the invention. Custom antibody and aptamer arrays are commercially available, for example, from Creative Biolabs (Shirley, NY).

It will be appreciated that the protease (e.g. trypsin) digestion will by definition result in a population of peptides cleaved from larger proteins and peptides present in a whole protein extract of the sample. Thus, in some embodiments, some of the AMP peptides identified by the proteomics approach are derived from the same, larger amino acid sequence, and there can be more than a single representative AMP sequence identified for each AMP (see, for example, Table S2).

In order to establish the proxy for a given physiological state (or states) of the tissue, the relationship between the AMP profile of the tissue and that of the exudate, secretion or excretion of the tissue is characterized, identifying shared AMPs. In some embodiments, the AMPs having shared abundance are determined according to the statistical significance of their correlation using statistical analysis, for example, Procrustes analysis.

Once a set (plurality, group) of AMPs having shared abundance in the tissue and in the exudate, secretion or excretion of the tissue for a given physiological state of the tissue has been identified, that plurality of AMPs can be considered a proxy for the physiological state or states of the tissue.

In certain embodiments, the physiological state comprises a disease state. The disease state may include a disease microenvironment and the expression of genes within the microenvironment of the tissue. The disease state may include an immune state. The disease state may indicate resistance or sensitivity to a treatment. The disease state may indicate the severity of a disease. Diseases or pathogens that lead to a disease state may include, but are not limited to metabolic diseases, cancer, an autoimmune disease, an inflammatory disease, or an infection. In specific embodiments, the disease state of the tissue is affected by a dysbiosis.

As used herein, the term “dysbiosis” refers to the disruption of the balanced microbial composition and collective functions of the microbiome inhabiting a subject or inhabiting a particular tissue (e.g. intestine) in a subject. The term typically refers to a decrease in beneficial microbial populations relative to deleterious microbial populations, or a change in the ratio of those populations such that microbial species that are normally only present in small numbers proliferate to a degree whereby they are present at elevated numbers. Gut dysbiosis can be a pathological imbalance in a microbial community characterized by a shift in the composition, diversity or function of microbial species, which can result in, or reflect the presence of a disease. Dysbiosis has been associated with a multitude of intestinal and extra-intestinal health-related outcomes in humans, including inflammatory bowel disease (IBD), autoimmune disease (e.g. systemic lupus erythmatosis SLE, multiple sclerosis MS, rheumatoid arthritis RA), cardiometabolic disease (e.g. atherosclerosis), metabolic disease (e.g. obesity, type 2 diabetes, non-alcoholic fatty liver disease NAFLD and non-alcoholic steatohepatitis NASH), cancer (e.g. colorectal cancer, hepatocellular carcinoma, breast cancer) and neurodegenerative disorders (e.g. Amyotropic Lateral Sclerosis, ALS, Parkinson's disease, autism spectrum disorder).

Thus, in some embodiments, the physiological state of the tissue is affected by cancer. Examples of cancer include but are not limited to carcinoma, lymphoma, blastoma, sarcoma, and leukemia or lymphoid malignancies. More particular examples of such cancers include without limitation: squamous cell cancer (e.g., epithelial squamous cell cancer), lung cancer including small-cell lung cancer, non-small cell lung cancer, adenocarcinoma of the lung, squamous carcinoma of the lung and large cell carcinoma of the lung, cancer of the peritoneum, hepatocellular cancer, gastric or stomach cancer including gastrointestinal cancer, pancreatic cancer, glioma, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as CNS cancer, melanoma, head and neck cancer, bone cancer, bone marrow cancer, duodenum cancer, oesophageal cancer, thyroid cancer, or hematological cancer.

Other non-limiting examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumours, Breast Cancer, Cancer of the Renal Pelvis and Urethra, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Glioblastoma, Childhood Cerebellar Astrocytoma, Childhood Cerebral Astrocytoma, Childhood Extracranial Germ Cell Tumours, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumours, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumours, Exocrine Pancreatic Cancer, Extracranial Germ Cell Tumour, Extragonadal Germ Cell Tumour, Extrahepatic Bile Duct Cancer, Eye Cancer, Female Breast Cancer, Gaucher's Disease, Gallbladder Cancer, Gastric Cancer, Gastrointestinal Carcinoid Tumour, Gastrointestinal Tumours, Germ Cell Tumours, Gestational Trophoblastic Tumour, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumour, Ovarian Low Malignant Potential Tumour, Pancreatic Cancer, Paraproteinemias, Purpura, Parathyroid Cancer, Penile Cancer, Pheochromocytoma, Pituitary Tumour, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Urethra Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumours, T-Cell Lymphoma, Testicular Cancer, Thymoma, Thyroid Cancer, Transitional Cell Cancer of the Renal Pelvis and Urethra, Transitional Renal Pelvis and Urethra Cancer, Trophoblastic Tumours, Urethra and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, or Wilms' Tumour. In some embodiments, the physiological state of the tissue is affected by cancer of the gastro-intestinal system, liver cancer and/or breast cancer. In other specific embodiments the physical state of the tissue is affected by colorectal cancer, gastric cancer, esophageal cancer, cholangiocarcinoma and gastrointestinal lymphoma.

In some embodiments, the physiological state of the tissue is affected by an auto-immune disease. As used throughout the present specification, the terms “autoimmune disease” or “autoimmune disorder” used interchangeably refer to a diseases or disorders caused by an immune response against a self-tissue or tissue component (self-antigen) and include a self-antibody response and/or cell-mediated response. The terms encompass organ-specific autoimmune diseases, in which an autoimmune response is directed against a single tissue, as well as non-organ specific autoimmune diseases, in which an autoimmune response is directed against a component present in two or more, several or many organs throughout the body.

Non-limiting examples of autoimmune diseases include but are not limited to acute disseminated encephalomyelitis (ADEM); Addison's disease; ankylosing spondylitis; antiphospholipid antibody syndrome (APS); aplastic anemia; autoimmune gastritis; autoimmune hepatitis; autoimmune thrombocytopenia; Behcet's disease; coeliac disease; dermatomyositis; diabetes mellitus type I; Goodpasture's syndrome; Graves' disease; Guillain-Barre syndrome (GBS); Hashimoto's disease; idiopathic thrombocytopenic purpura; inflammatory bowel disease (IBD) including Crohn's disease and ulcerative colitis; mixed connective tissue disease; multiple sclerosis (MS); myasthenia gravis; opsoclonus myoclonus syndrome (OMS); optic neuritis; Ord's thyroiditis; pemphigus; pernicious anaemia; polyarteritis nodosa; polymyositis; primary biliary cirrhosis; primary myoxedema; psoriasis; rheumatic fever; rheumatoid arthritis; Reiter's syndrome; scleroderma; Sjogren's syndrome; systemic lupus erythematosus; Takayasu's arteritis; temporal arteritis; vitiligo; warm autoimmune hemolytic anemia; or Wegener's granulomatosis. In particular embodiments, the physiological state of the tissue is affected by systemic lupus erythmatosis SLE, multiple sclerosis MS and/or rheumatoid arthritis RA, Inflammatory Bowel Disease (Crohn's disease and ulcerative colitis) and celiac disease.

The disease may be an allergic inflammatory disease. The allergic inflammatory disease may be selected from the group consisting of asthma, allergy, allergic rhinitis, allergic airway inflammation, atopic dermatitis (AD), chronic obstructive pulmonary disease (COPD), inflammatory bowel disease (IBD), multiple sclerosis, arthritis, psoriasis, eosinophilic esophagitis, eosinophilic pneumonia, eosinophilic psoriasis, hypereosinophilic syndrome, graft-versus-host disease, uveitis, cardiovascular disease, pain, multiple sclerosis, lupus, vasculitis, chronic idiopathic urticaria and Eosinophilic Granulomatosis with Polyangiitis (Churg-Strauss Syndrome). The asthma may be selected from the group consisting of allergic asthma, non-allergic asthma, severe refractory asthma, asthma exacerbations, viral-induced asthma or viral-induced asthma exacerbations, steroid resistant asthma, steroid sensitive asthma, eosinophilic asthma and non-eosinophilic asthma. The allergy may be to an allergen selected from the group consisting of foods, pollen, mold, dust mites, animals, and animal dander. IBD may comprise a disease selected from the group consisting of ulcerative colitis (UC), Crohn's Disease, collagenous colitis, lymphocytic colitis, ischemic colitis, diversion colitis, Behcet's syndrome, infective colitis, indeterminate colitis, and other disorders characterized by inflammation of the mucosal layer of the large intestine or colon. In other embodiments, the disease may be an internal organ disorder which is associated with inflammation of the gut, such as primary sclerosing cholangitis (PSC). The arthritis may be selected from the group consisting of osteoarthritis, rheumatoid arthritis and psoriatic arthritis.

In some embodiments, the physiological state of the tissue can be affected by an infection, including, but not limited to bacterial, viral, fungal and protozoan infections. Examples of pathogenic bacteria that can affect the physiological state of the tissue include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginate, Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtherias, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia hemolytica, Moraxella catarrhalis, Morganella sp., Mobiluncus sp., Micrococcus sp., Mycobacterium sp. (such as Mycobacterium leprae, Mycobacterium tuberculosis (MTB), Mycobacterium paratuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia.

In certain exemplary embodiments, the physiological state of the tissue is affected by a fungal infection. Examples of fungi that can affect the state of the tissue include without limitation any one or more of (or any combination of), Aspergillus, Blastomyces, Candidiasis, Coccidiodomycosis, Cryptococcus neoformans, Cryptococcus gatti, Histoplasma, Mucroymcosis, Pneumocystis, Sporothrix, fungal eye infections ringworm, Exserohilum, and Cladosporium.

In certain embodiments, the fungus is a yeast. Examples of yeast that can affect the state of the tissue include without limitation one or more of (or any combination of), Aspergillus species, a Geotrichum species, a Saccharomyces species, a Hansenula species, a Candida species, a Kluyveromyces species, a Debaryomyces species, a Pichia species, or combination thereof. In certain exemplary embodiments, the fungus is a mold. Exemplary molds include, but are not limited to, a Penicillium species, a Cladosporium species, a Byssochlamys species, or a combination thereof.

In certain example embodiments, the pathogen may be a virus. The virus may be a DNA virus, a RNA virus, or a retrovirus.

In certain embodiments, the pathogen may be a protozoon. Examples of protozoa include without limitation any one or more of (or any combination of), Euglenozoa, Heterolobosea, Diplomonadida, Amoebozoa, Blastocystic, and Apicomplexa. Euglenoza include, but are not limited to, Trypanosoma cruzi (Chagas disease), T. brucei gambiense, T. brucei rhodesiense, Leishmania braziliensis, L. infantum, L. mexicana, L. major, L. tropica, and L. donovani. Heterolobosea include, but are not limited to, Naegleria fowleri. Diplomonadid include, but are not limited to, Giardia intestinalis (G. lamblia, G. duodenalis). Amoebozoa include, but are not limited to, Acanthamoeba castellanii, Balamuthia madrillaris, Entamoeba histolytica. Blastocystis include, but are not limited to, Blastocystic hominis. Apicomplexa include, but are not limited to, Babesia microti, Cryptosporidium parvum, Cyclospora cayetanensis, Plasmodium falciparum, P. vivax, P. ovale, P. malariae, and Toxoplasma gondii.

In specific embodiments, the physiological state of the tissue can be affected by an infection, including, but not limited to C difficil, enterohemorrhagic E coli, salmonella, intestinal TB, intestinal CMV, helicobacter, campylobacter, rotavirus, norovirus.

While applying the novel criteria for determining an AMP to the peptides of murine gut mucosal tissue, the present inventors have surprisingly uncovered previously unrecognized AMPs, fulfilling the two or more of criteria (b)(i)-(vi). Thus, there is provided an antimicrobial peptide selected from the group consisting of Ace, Adipoq, Asah2, Azgp 1, Cd177, Clec3b, Clu, Csta, Cstb, Cstd, Cste, Cstl, Habp2, Itgam, Itgb2, Manf, Mmp9, Pla2g4c, Pla2g15, Reg1, Reg2, S100a13, Retnlg, Mptxl and Chil3. In some embodiments, Retnlg is a resistin-like AMP, and possesses increased abundance in the gut in the physiological state of infection and/or inflammation. In some embodiments, Retnlg AMP is at least 75, at least 80, at least 85, at least 90, at least 91, 92, 93, 94, 95, 96, 97, 98, 99 or 100% identical to the amino acid sequence of SEQ ID NO: 20. In some embodiments, the resistin-like AMP is the human ortholog of murine Retnlg AMP.

In some embodiments, Mptxl is a pentraxin-like AMP, and possesses increased abundance in the gut with commensal colonization of the gut, and decreased abundance in the physiological state of gut infection and/or inflammation. In some embodiments, Mptxl AMP is at least 75, at least 80, at least 85, at least 90, at least 91, 92, 93, 94, 95, 96, 97, 98, 99 or 100% identical to the amino acid sequence of SEQ ID NO: 22. In some embodiments, the pentraxin-like AMP is the human ortholog of murine Mptx-1 AMP.

In some embodiments, Chil3 is a bacteria-binding lectin-like AMP, and possesses increased abundance in the gut with commensal colonization of the gut, as well as abundance in the physiological state of gut infection and/or inflammation. In some embodiments, Chil3 AMP is at least 75, at least 80, at least 85, at least 90, at least 91, 92, 93, 94, 95, 96, 97, 98, 99 or 100% identical to the amino acid sequence of SEQ ID NO: 15. In some embodiments, the bacterial-binding lectin-like AMP is the human ortholog of murine Chil3 AMP.

In some embodiments, Ace is an angiotensin converting enzyme 2 AMP, and possesses increased abundance in the gut in the physiological state of inflammation. In some embodiments, Ace AMP is at least 75, at least 80, at least 85, at least 90, at least 91, 92, 93, 94, 95, 96, 97, 98, 99 or 100% identical to the amino acid sequence of SEQ ID NO: 17. In some embodiments, the angiotensin converting enzyme 2 AMP is the human ortholog of murine Ace AMP.

In some embodiments, Adipoq is an adiponectin AMP, and possesses increased abundance in the gut in the physiological state of inflammation. In some embodiments, Adipoq AMP is at least 75, at least 80, at least 85, at least 90, at least 91, 92, 93, 94, 95, 96, 97, 98, 99 or 100% identical to the amino acid sequence of SEQ ID NO: 27. In some embodiments, the adiponectin AMP is the human ortholog of murine Adipoq AMP.

In some embodiments, Asah2 is a neutral ceramidase AMP, and possesses increased abundance in the gut in the physiological state of inflammation. In some embodiments, Asah2 AMP is at least 75, at least 80, at least 85, at least 90, at least 91, 92, 93, 94, 95, 96, 97, 98, 99 or 100% identical to the amino acid sequence of SEQ ID NO: 18. In some embodiments, the neutral ceramidase AMP is the human ortholog of murine Asah2 AMP.

In some embodiments, Azgpl is a zinc-alpha-2 glycoprotein AMP, and possesses increased abundance in the gut in the physiological state of inflammation. In some embodiments, Azgpl AMP is at least 75, at least 80, at least 85, at least 90, at least 91, 92, 93, 94, 95, 96, 97, 98, 99 or 100% identical to the amino acid sequence of SEQ ID NO: 26. In some embodiments, the zinc-alpha-2 glycoprotein AMP is the human ortholog of murine Azgpl AMP.

In some embodiments, Cd177 is a CD177 antigen AMP, and possesses increased abundance in the gut in the physiological state of infection and/or inflammation. In some embodiments, CD177 AMP is at least 75, at least 80, at least 85, at least 90, at least 91, 92, 93, 94, 95, 96, 97, 98, 99 or 100% identical to the amino acid sequence of SEQ ID NO: 21. In some embodiments, the CD177 antigen AMP is the human ortholog of murine Cd177 AMP.

In some embodiments, Clu is a clusterin AMP, and possesses increased abundance in the gut with commensal colonization of the gut, as well as abundance in the physiological state of gut infection and/or inflammation. In some embodiments, Clu AMP is at least 75, at least 80, at least 85, at least 90, at least 91, 92, 93, 94, 95, 96, 97, 98, 99 or 100% identical to the amino acid sequence of SEQ ID NO: 6. In some embodiments, the clusterin AMP is the human ortholog of murine Clu AMP.

In some embodiments, Csta (also known as CYTA) is a Cystatin-A AMP, and possesses increased abundance in the gut in the physiological state of bacterial infection. In some embodiments, Csta AMP is at least 75, at least 80, at least 85, at least 90, at least 91, 92, 93, 94, 95, 96, 97, 98, 99 or 100% identical to the amino acid sequence of SEQ ID NO: 12. In some embodiments, the Cystatin-A AMP is the human ortholog of murine Csta AMP.

In some embodiments, Cstb (also known as CYTB) is a cystatin-B AMP, and possesses increased abundance in the gut in the physiological state of inflammation. In some embodiments, Cstb AMP is at least 75, at least 80, at least 85, at least 90, at least 91, 92, 93, 94, 95, 96, 97, 98, 99 or 100% identical to the amino acid sequence of SEQ ID NO: 10. In some embodiments, the Cystatin B AMP is the human ortholog of murine Cstb AMP.

In some embodiments, Ctsd is a cathepsin-D AMP, and possesses increased abundance in the gut in the physiological state of inflammation. In some embodiments, Ctsd AMP is at least 75, at least 80, at least 85, at least 90, at least 91, 92, 93, 94, 95, 96, 97, 98, 99 or 100% identical to the amino acid sequence of SEQ ID NO: 3. In some embodiments, the cathepsin-D AMP is the human ortholog of murine Cst-D AMP.

In some embodiments, Ctse is a cathepsin E AMP, and possesses increased abundance in the gut in the physiological state of infection. In some embodiments, Ctse AMP is at least 75, at least 80, at least 85, at least 90, at least 91, 92, 93, 94, 95, 96, 97, 98, 99 or 100% identical to the amino acid sequence of SEQ ID NO: 23. In some embodiments, the cathepsin E AMP is the human ortholog of murine Ctse AMP.

In some embodiments, Ctsl is a cathepsin L1 AMP, and possesses increased abundance in the gut in the physiological state of infection and/or inflammation. In some embodiments, Ctsl AMP is at least 75, at least 80, at least 85, at least 90, at least 91, 92, 93, 94, 95, 96, 97, 98, 99 or 100% identical to the amino acid sequence of SEQ ID NO: 4. In some embodiments, the cathepsinL AMP is the human ortholog of murine Ctsl AMP.

In some embodiments, Habp2 is a hyaluronan binding protein 2 AMP, and possesses increased abundance in the gut in the physiological state of inflammation. In some embodiments, Habp2 AMP is at least 75, at least 80, at least 85, at least 90, at least 91, 92, 93, 94, 95, 96, 97, 98, 99 or 100% identical to the amino acid sequence of SEQ ID NO: 16. In some embodiments, the hyaluronan binding protein 2 AMP is the human ortholog of murine Habp2 AMP.

In some embodiments, Itgam is an integrin alpha-M AMP, and possesses increased abundance in the gut in the physiological state of inflammation. In some embodiments, Itgam AMP is at least 75, at least 80, at least 85, at least 90, at least 91, 92, 93, 94, 95, 96, 97, 98, 99 or 100% identical to the amino acid sequence of SEQ ID NO: 5. In some embodiments, the integrin alpha M AMP is the human ortholog of murine Itgam AMP.

In some embodiments, Itgb2 is an integrin beta 2 AMP, and possesses increased abundance in the gut in the physiological state of inflammation. In some embodiments, Itgb2 AMP is at least 75, at least 80, at least 85, at least 90, at least 91, 92, 93, 94, 95, 96, 97, 98, 99 or 100% identical to the amino acid sequence of SEQ ID NO: 11. In some embodiments, the integrin beta 2 AMP is the human ortholog of murine Itgb2 AMP.

In some embodiments, Manf is a mesencephalic astrocyte-derived neuroptophic factor AMP, and possesses increased abundance in the gut in the physiological state of inflammation. In some embodiments, Manf AMP is at least 75, at least 80, at least 85, at least 90, at least 91, 92, 93, 94, 95, 96, 97, 98, 99 or 100% identical to the amino acid sequence of SEQ ID NO: 13. In some embodiments, the mesencephalic astrocyte-derived neuroptophic factor AMP is the human ortholog of murine Manf AMP.

In some embodiments, Mmp9 is a matrix metalloproteinase 9 AMP, and possesses increased abundance in the gut in the physiological state of infection and/or inflammation. In some embodiments, Mmp9 AMP is at least 75, at least 80, at least 85, at least 90, at least 91, 92, 93, 94, 95, 96, 97, 98, 99 or 100% identical to the amino acid sequence of SEQ ID NO: 24. In some embodiments, the matrix metalloproteinase 9 AMP is the human ortholog of murine Mmp9 AMP.

In some embodiments, Pla2g4c is a cytosolic phospholipase A2 gamma AMP, and possesses increased abundance in the gut in the physiological state of infection and/or inflammation. In some embodiments, Pla2g4c AMP is at least 75, at least 80, at least 85, at least 90, at least 91, 92, 93, 94, 95, 96, 97, 98, 99 or 100% identical to the amino acid sequence of SEQ ID NO: 19. In some embodiments, the cytosolic phospholipase A2 gamma AMP is the human ortholog of murine Pla2g4c AMP.

In some embodiments, Pla2g15 is a group XV phospholipase A2 AMP, and possesses increased abundance in the gut in the physiological state of inflammation. In some embodiments, Pla2g15 AMP is at least 75, at least 80, at least 85, at least 90, at least 91, 92, 93, 94, 95, 96, 97, 98, 99 or 100% identical to the amino acid sequence of SEQ ID NO: 14. In some embodiments, the group XV phospholipase A2 AMP is the human ortholog of murine Pla2g15 AMP.

In some embodiments, Reg1 is a lithostathine-1 AMP, and possesses increased abundance in the gut in the physiological state of infection and/or inflammation. In some embodiments, Reg1 AMP is at least 75, at least 80, at least 85, at least 90, at least 91, 92, 93, 94, 95, 96, 97, 98, 99 or 100% identical to the amino acid sequence of SEQ ID NO: 7. In some embodiments, the lithostathine-1 AMP is the human ortholog of murine Reg1 AMP.

In some embodiments, Reg2 is a lithostathine-2 AMP, and possesses increased abundance in the gut in the physiological state of infection and/or inflammation. In some embodiments, Reg2 AMP is at least 75, at least 80, at least 85, at least 90, at least 91, 92, 93, 94, 95, 96, 97, 98, 99 or 100% identical to the amino acid sequence of SEQ ID NO: 8. In some embodiments, the lithostathine-2 AMP is the human ortholog of murine Reg2 AMP.

In some embodiments, S100a13 is a S100-A13 calcium binding protein AMP, and possesses increased abundance in the gut in the physiological state of infection and/or inflammation. In some embodiments, S100a13 AMP is at least 75, at least 80, at least 85, at least 90, at least 91, 92, 93, 94, 95, 96, 97, 98, 99 or 100% identical to the amino acid sequence of SEQ ID NO: 25. In some embodiments, the S100-A13 AMP is the human ortholog of murine S100a13 AMP.

The term “peptide” as used herein encompasses native peptides (either degradation products, synthetically synthesized peptides or recombinant peptides) and peptidomimetics (typically, synthetically synthesized peptides), as well as as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated amide bonds (—N(CH3)-CO—), ester bonds (—C(═O)—O—), ketomethylene bonds (—CO—CH2-), sulfinylmethylene bonds (—S(═O)—CH2-), α-aza bonds (—NH—N(R)—CO—), wherein R is any alkyl (e.g., methyl), amine bonds (—CH2-NH—), sulfide bonds (—CH2-S—), ethylene bonds (—CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides of some embodiments of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1), and non-conventional or modified amino acids (e.g., synthetic, Table 2) which can be used with some embodiments of the invention.

TABLE 1

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid | Xaa | X |
| as above | | |

TABLE 2

| Non-conventional amino | | Non-conventional amino | |
|---|---|---|---|
| acid | Code | acid | Code |
| ornithine | Orn | hydroxyproline | Hyp |
| α-aminobutyric acid | Abu | aminonorbornyl- | Norb |
| | | carboxylate | |
| D-alanine | Dala | aminocyclopropane- | Cpro |
| | | carboxylate | |
| D-arginine | Darg | N-(3- | Narg |
| | | guanidinopropyl)glycine | |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl- | Nmhphe |
| | | homophenylalanine | |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino-α- | Nmaabu |
| | | methylbutyrate | |
| α-aminoisobutyric acid | Aib | N-methyl-α- | Nmaib |
| | | aminoisobutyrate | |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl- | Mhphe |
| | | homophenylalanine | |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2- | Nbhm | N-(N-(2,2-diphenylethyl) | Nnbhm |
| diphenylethyl)glycine | | carbamylmethyl-glycine | |
| N-(3,3- | Nbhe | N-(N-(3,3-diphenylpropyl) | Nnbhe |
| diphenylpropyl)glycine | | carbamylmethyl-glycine | |
| 1-carboxy-1-(2,2-diphenyl | Nmbc | 1,2,3,4- | Tic |
| ethylamino)cyclopropane | | tetrahydroisoquinoline-3- | |
| | | carboxylic acid | |
| phosphoserine | pSer | phosphothreonine | pThr |
| phosphotyrosine | pTyr | O-methyl-tyrosine | |
| 2-aminoadipic acid | | hydroxylysine | |

The peptides of some embodiments of the invention are preferably utilized in a linear form, although it will be appreciated that in cases where cyclicization does not severely interfere with peptide characteristics, cyclic forms of the peptide can also be utilized.

Since the present peptides are preferably utilized in therapeutics or diagnostics which require the peptides to be in soluble form, the peptides of some embodiments of the invention preferably include one or more non-natural or natural polar amino acids, including but not limited to serine and threonine which are capable of increasing peptide solubility due to their hydroxyl-containing side chain.

The peptides of some embodiments of the invention are preferably utilized in a linear form, although it will be appreciated that in cases where cyclicization does not severely interfere with peptide characteristics, cyclic forms of the peptide can also be utilized.

The peptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide compounds of some embodiments of the invention involves solid phase peptide synthesis.

Large scale peptide synthesis is described by Andersson Biopolymers 2000; 55(3):227-50.

It will be appreciated that the methods of the invention can be used to diagnose and monitor physiological and/or disease states of the tissue, or of the organ in which the tissue resides, or of the subject.

According to this aspect of the present invention, the subject from whom the sample of the exudate, secretion or excretion of the tissue has been obtained can be diagnosed according to the levels of the plurality AMP. If the test AMP profile comprises AMPs having shared abundance with the corresponding plurality of AMPs in the tissue in a pathological physiological state, it is indicative that the subject has a disease.

Alternatively, or additionally, if the test AMP profile comprises AMPs which have shared abundance with the corresponding plurality of AMPs in the tissue in a healthy physiological state, it is indicative that the subject does not have a disease.

In order to diagnose a subject as having a disease, typically at least 1, more preferably at least 5, more preferably at least 10, more preferably at least 20, more preferably at least 30, more preferably at least 40, more preferably at least 50, more preferably about 100 of the AMPs have a shared abundance similar to the AMPs from the tissue in a pathological state, indicating a disease. In some embodiments, about 5-25, about 1-30, about 5-75, about 15-100, about 20-95, about 10-45, about 20-80, about 25-65 of the AMPs have a shared abundance similar to the AMPs from the tissue in a pathological state, indicating a disease.

For example, when a test subject's fecal AMP profile has increased levels of one or more PMN-associated AMPs including, but not limited to Prtn3, Azul, Ltf, Ctsg, S100a8, S100a9, S100a12, and/or AMPs PRB3, PGC, APCS, PGLYRP1, MPO, PRSS2 or ELANE along with the decreased abundance of one or more intestinal epithelial AMPs including, but not limited to Lgals4, Gp2, Asah2, this is indicative that the subjects gut tissue has a diseased physiological state, and that the test subject has active Crohn's disease.

In another example, when the test subject's fecal AMP profile has increased levels of at least 2, at least 3 or all 5 of S100a8, CEACAM8, Mpo (Perm), Prtn3 and IgA2, along with a decreased abundance of Muc2, Muc4, Muc12 and CECAM6, this is indicative that the subject's gut tissue has a diseased physiological state, and that the test subject has Ulcerative Colitis. When the test subject's fecal AMP profile has increased levels of at least 2, at least 3 or all 5 of S100a8, S100a9, CEACAM8, Mpo (Perm) and Prtn3, this is indicative that the subject's gut tissue has a diseased physiological state, and that the test subject has Crohn's Disease.

In still another example, when the test subject's fecal AMP profile has increased levels of at least 2, at least 3 or at least 5 or more of S100a8, S100a9, CEACAM8, Ecp2, Azu, Ctsg, Mpo (Perm), alpha-1-antichymotrypsin, Prtn3, Exp, Rnas4, Elane, CtsA, orosomucoid or alpha-amylase, along with a decreased abundance of Muc12, this is indicative that the subject's gut tissue has a diseased physiological state, and that the test subject has Primary Sclerosing Cholangitis.

In yet another example, when a test subject's fecal AMP profile has increased levels of at least two, at least 3, at least 5, at least 10 of Ltf, Ngp, S100a9, Lcn2, Cd177, Chil3, S100a8, Mpo, Camp, Itgb2, Itgam, Retnlg, Elmo1, Ido1, Elane, Epx, Prg2, Mmp9, Ear2, Lcp 1, Fam49b, B2 m, Nos2, Fn1, Ctsg, Pon2, Apoe, Psap, Isg15, Pglyrp1, Gzma, Pltp, Apoa1, Arg1 and Gsdmdc 1, this is indicative that the subjects gut tissue has a diseased physiological state, and that the test subject has a late-stage bacterial gut infection. A fecal AMP profile having increased levels of at least two, at least 3, at least 5, at least 10 of Ltf, Ngp, S100a9, Lcn2, Cd177, Chil3, S100a8, Mpo, Camp, Itgb2, Itgam, Retnlg, Elmo1, Ido1, Elane, Dmbt1, Epx, Prg2, Pon1, Apoa1, Slpi, Mmp9, Ctsg, this is indicative that the subjects gut tissue has a diseased physiological state, and that the test subject has an early to mid-stage bacterial gut infection. Thus, the methods of the invention can be used to determine the stage of a disease or condition, to monitor treatment and/or provide prognostic assessment of a physiological state of a tissue or of a disease or condition.

It will be appreciated that wherein the gut tissue is human gut tissue, the AMPs are human AMPs or human orthologs of the already-identified murine AMPs.

It will be appreciated that the AMP binding moieties of some embodiments of the invention which are described hereinabove for detecting the plurality of AMPs may be included in a diagnostic kit/article of manufacture preferably along with appropriate instructions for use and labels indicating FDA approval for use in determining a physiological state of a tissue of a subject, or for diagnosing and/or monitoring a disease or condition and/or severity thereof in the subject.

Such a kit can include, for example, at least one container including at least two of the above described diagnostic agents (e.g., AMP-binding antibodies, aptamers) and an imaging reagent packed in another container (e.g., enzymes, secondary antibodies, buffers, chromogenic substrates, fluorogenic material). The kit may also include appropriate buffers and preservatives for improving the shelf-life of the kit.

Thus, according to some embodiments of the invention, there is provided a kit for determining the physiological state of a tissue, comprising greater than 10 and fewer than 100 AMP-binding moieties, each binding moiety binding to a different AMP having shared abundance in the tissue and in an exudate, secretion or excretion of the tissue in a given physiological state.

In some embodiments, the kit comprises AMP binding moieties selected from the group consisting of antibodies, aptamers and ligands of the greater than 10 and fewer than 100 AMPs.

The present inventors have shown the association of increased abundance of some AMPs in the gut (and, concomitantly, in the feces) with changes in the physiological state of the gut tissue (e.g. inflammatory states, IBD), indicating that the anti-microbial properties of some of the gut-associated AMPs could be active components of the successful inhibition of cytotoxic and/or inflammatory processes and agents of the gut. Thus, the present invention, in some embodiments, also envisages therapeutic application of the AMPs.

Thus, in some aspects of some embodiments, there is provided a method for treating or preventing a physiological state of a tissue comprising administering to the tissue at least one AMP associated with the physiological state of the tissue. In some embodiments, the tissue is gut tissue and the AMP is a gut-associated AMP selected from the group consisting of Camp, Lnc2, Ltf, Mpo, Ngp, Prtn 3, Elane, Ctsg, Pglyrp1, S100a8, S100a9, Ear2, Epx and Prg2, Pla2g 1b, Reg3a, Reg3g and Reg4. In some embodiments, there is provided at least one AMP for prevention and/or treatment of a diseased physiological state of a tissue. In specific embodiments, the tissue is gut tissue and the diseased physiological state is inflammation or bacterial infection. In other embodiments, the tissue is gut tissue and the physiological state is dysbiosis, and the at least one AMP is administered to modulate the gut microbiome towards a healthy physiological state. In some embodiments, the at least one gut-associated AMP can be 2, 3, 4, 5, 6, 7, 8, 9, 10, 11, 12, 13, 14, 15, 16, 17 or all 18 of the group's gut-associated AMPs.

In particular embodiments, the tissue is human gut tissue, and the at least one AMP is a human AMP, or a human ortholog of an indicated murine AMP selected from the group consisting of Camp, Lnc2, Ltf, Mpo, Ngp, Prtn 3, Elane, Ctsg, Pglyrp1, S100a8, S100a9, Ear2, Epx and Prg2, Pla2g1b, Reg3a, and Reg4.

In some embodiments, the tissue is human gut tissue and the diseased physiological state is inflammatory bowel disease (IBD), and the at least one AMP is a human AMP selected from the group consisting of PRTN3, AZU, S100A8, S100A9, S100A12, CTSG, LTF, PRB3, PGC, CP, APCS, PGLYRP1, MPO, PRSS2, RNASE2, ELANE, ORM1, PLA2G1B, SOD2 and LGALS4. In particular embodiments, the at least one AMP is selected from the group consisting of SEQ ID Nos. 28-47. In some emodiments, the at least one human AMP can be 2, 3, 4, 5, 6, 7, 8, 9, 10, 11, 12, 13, 14, 15, 16, 17, 18, 19 or all 20 of the group's human AMPs.

The AMPs of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the AMPs accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections. For gut-related treatments, oral, rectal and intestinal administration is preferred.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (AMPs) effective to prevent, alleviate or ameliorate symptoms of a disorder (e.g., gut inflammation) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings at any age which suffer from the pathology. Preferably, this term encompasses individuals who are at risk to develop the pathology.

As used herein the phrase “treatment regimen” refers to a treatment plan that specifies the type of treatment, dosage, schedule and/or duration of a treatment provided to a subject in need thereof (e.g., a subject diagnosed with a pathology). The selected treatment regimen can be an aggressive one which is expected to result in the best clinical outcome (e.g., complete cure of the pathology) or a more moderate one which may relief symptoms of the pathology yet results in incomplete cure of the pathology. It will be appreciated that in certain cases the more aggressive treatment regimen may be associated with some discomfort to the subject or adverse side effects (e.g., a damage to healthy cells or tissue). The type of treatment can include a surgical intervention (e.g., removal of lesion, diseased cells, tissue, or organ), a cell replacement therapy, an administration of a therapeutic drug (e.g., receptor agonists, antagonists, hormones, chemotherapy agents) in a local or a systemic mode, an exposure to radiation therapy using an external source (e.g., external beam) and/or an internal source (e.g., brachytherapy) and/or any combination thereof. The dosage, schedule and duration of treatment can vary, depending on the severity of pathology and the selected type of treatment, and those of skills in the art are capable of adjusting the type of treatment with the dosage, schedule and duration of treatment.

It is expected that during the life of a patent maturing from this application many relevant methods for identifying antimicrobial peptides and proteins will be developed and the scope of the AMP is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Animal Model

Mice. All animal studies were approved by the Weizmann Institute of Science Institutional Animal Care and Use committee (IACUC), application number 10100119-2. Specific-pathogen-free (SPF) C57BL/6 mice were purchased from Harlan Envigo and acclimatized to the animal facility environment for 2 weeks before starting experiments. C57BL/6 Germ-free (GF) mice were born and kept in the GF facility of the Weizmann Institute of Science and routinely monitored for sterility. All mice were maintained under a strict 12-h light-dark cycle, with lights on at 6 am and off at 6 pm. Eight-week-old mice were used in all experiments.

Dissection of different regions along the murine gastrointestinal tract. Age- and gender-matched SPF and GF mice were sacrificed by CO2 asphyxiation, and a laparotomy was performed by employing a vertical midline incision. Three different parts of the digestive tract were exposed and harvested: the ileum (the distal third of the small intestine), the cecum, and the distal part of the colon. For each section, the luminal contents were removed, the remaining tissue was cut open longitudinally, rinsed with sterile phosphate buffered saline (PBS−/−) for three times, and collected for mucosal proteomics.

Segmental filamentous bacteria (SFB) mono-colonization in GF mice. Fresh frozen cecal contents from mSFB-monocolonized or rSFB-monocolonized were resuspended in sterile PBS−/− in a vinyl isolator, filtered through a 100-μm cell strainer, and immediately transferred to GF C57BL/6 mice by oral gavage (200 μl resuspension/mouse). GF mice orally inoculated with sterile PBS−/− were used as negative controls. After 2 weeks of colonization, mice were sacrificed, terminal ileal mucosal samples were harvested and divided into three aliquots. Finally, the samples were subjected to proteomics, metagenomics, and RNA-sequencing, respectively.

Citrobacter rodentium infection in SPF mice. A kanamycin-resistant C. rodentium strain, DBS100 (ICC180), was used for infection. SPF mice were infected by oral gavage with 200 μl of bacterial solution cultured overnight, containing approximately 1×10⁹ colony-forming units (CFU). Both the distal colonic mucosal and stool samples were collected from mice without infection (day0), during the peak of infection (day7) and at the infection recovery phase (day14), and subjected to proteomics, microbiome sequencing and RNA-sequencing (only for colon samples). The pathogen load was quantified by counting the stool CFU. In detail, stool samples were weighed, homogenized in sterile PBS−/−, serially diluted in PBS−/− and plated on LB kanamycin plates. After incubating overnight at 37° C., bacterial CFUs were counted and normalized to the stool weight.

Dextran sulfate sodium (DSS)-induced colitis. SPF mice were treated with 2% (weight/volume) DSS (molecular weight, 36,000-50,000 Da; MP Biomedicals, Solon, OH) added to the drinking water for 7 days followed by the resumption of regular water. Disease severity was monitored by weighing the mice daily. Fresh stool samples were collected before DSS treatment (day 0), at the early inflammatory phase (day3, day5), late inflammatory phase (dayl6) and the end of the weight recovery phase (day31), and subjected to proteomics and metagenomic sequencing. Colon samples were harvested at day5 for histology by hematoxylin-eosin staining, as well as for flow cytometry (see below).

Interleukin-18 (IL-18) supplementation in GF mice. GF mice were injected intraperitoneally with recombinant IL-18 (MBL, Cat #B004-2, powder dissolved in sterile PBS−/) for 5 days at a dose of 1 μg/mouse/injection, twice daily. GF mice injected with sterile PBS−/− only were used as negative controls. Distal colonic mucosal samples were harvested and subjected to proteomics. During the experiment, the mice were kept sterile using a cage-autonomous system.

Flow Cytometry

Colon tissues were dissected from mice treated with DSS at day5 or untreated mice. Colonic contents were removed by extensive washing, and the colonic epithelial cells were dissociated in Hanks' Balanced Salt Solution (HBSS−/−) containing 2 mM EDTA at 4° C. for 15 min for 3 times. Following extensive shaking, the epithelial fractions were combined, pelleted and resuspended in cold PBS−/−, and stained with antibodies against EpCAM and CD45 (Biolegend, San Diego, CA) for 30 min on ice, followed by staining with the viability dye DAPI. The remaining colon tissue was then digested with DNase I and collagenase in fetal calf serum (FCS)-containing HBSS−/− at 37° C. for 40 min, filtered, pelleted and resuspended in PBS−/− containing 2% FCS. Single-cell suspensions were blocked and stained with antibodies against CD45, CD11b, Ly6G, MHCII for 30 min on ice, followed by staining with the viability dye DAPI. Stained cells were analyzed on a BD-LSR Fortessa cytometer and were analyzed with FlowJo v8 software. Total counts of specific live cell types (epithelial cells, neutrophils) were compared between different groups using Mann-Whitney U test.

Mass Spectrometry-Based Proteomics

Sample preparation. Samples were subjected to in-solution tryptic digestion using suspension trapping (S-trap). Briefly, mucosal tissues were first homogenized in cold sterile PBS−/−. After centrifugation, the clear supernatant containing soluble proteins was supplemented with lysis buffer to a final concentration of 5% SDS in 50 mM Tris-HCl pH 7.4. Mouse and human stool samples were directly homogenized in lysis buffer containing 5% SDS in 50 mM Tris-HCl pH 7.4. Lysates were then incubated at 96° C. for 5 min, followed by six cycles of 30 sec of sonication (Bioruptor Pico, Diagenode, USA). Protein concentration was measured using the BCA assay (Thermo Scientific, USA). An amount of 50 ug total protein was reduced with 5 mM dithiothreitol and alkylated with 10 mM iodoacetamide in the dark. Each sample was loaded onto S-Trap microcolumns (Protifi, USA) according to the manufacturer's instructions. After loading, samples were washed with 90:10% methanol/50 mM ammonium bicarbonate. Samples were then digested with trypsin (1:50 trypsin/protein) for 1.5 h at 47° C. The digested peptides were eluted using 50 mM ammonium bicarbonate. Trypsin was added to this fraction and incubated overnight at 37° C. Two more elutions were made using 0.2% formic acid and 0.2% formic acid in 50% acetonitrile. The three elutions were pooled together and vacuum-centrifuged to dryness. Stool samples were subjected to an additional cleaning step using solid-phase extraction (Oasis HBL, Waters, MA, USA) according to manufacturer instructions. For samples to be analyzed by parallel reaction monitoring (PRM), an equal amount of peptide mix containing 21 stable isotope-labeled (SIL) synthetic peptides (Table S2) was spiked into the digests. Samples were kept at −80° C. until further analysis.

Liquid chromatography. ULC/MS grade solvents were used for all chromatographic steps. Dry digested samples were dissolved in 97:3% H₂O/acetonitrile+0.1% formic acid. Each sample was loaded and analyzed using split-less nano-Ultra Performance Liquid Chromatography (10 kpsi nanoAcquity; Waters, Milford, MA, USA). The mobile phase was: A) H₂O+0.1% formic acid and B) acetonitrile+0.1% formic acid. Desalting of the samples was performed online using a Symmetry C18 reversed-phase trapping column (180 μm internal diameter, 20 mm length, 5 μm particle size; Waters). The peptides were then separated using an HSS T3 nano-column (75 μm internal diameter, 250 mm length, 1.8 μm particle size; Waters) at 0.35 μL/min. For label-free quantitative (LFQ) proteomic analysis, peptides were eluted from the column into the mass spectrometer using the following gradient: 4% to 25% B in 155 min, 25% to 90% B in 5 min, maintained at 90% for 5 min, and then back to initial conditions. For PRM analysis, the gradient was: 4% to 30% B in 97 min, 30% to 90% B in 5 min, maintained at 90% for 5 min, and then back to initial conditions.

Mass Spectrometry. The nanoUPLC was coupled online through a nanoESI emitter (10 μm tip; New Objective; Woburn, MA, USA) to a quadrupole orbitrap mass spectrometer (Q Exactive HFX or HF, Thermo Scientific) using a Flexion nanospray apparatus (Proxeon). For LFQ analysis, data were acquired in data-dependent acquisition (DDA) mode, using a Top10 method. MS1 resolution was set to 120,000 (at 200 m/z), mass range of 375-1650 m/z, AGC of 3e6, and maximum injection time was set to 60 msec. MS2 resolution was set to 15,000, quadrupole isolation 1.7 m/z, AGC of 1e5, dynamic exclusion of 45 sec, and maximum injection time of 60 msec. For PRM analysis, data was acquired in PRM mode with scheduled monitoring of 148 native (unlabeled) peptides and 21 SIL synthetic peptides, corresponding to 68 proteins (Table S2). MS1 resolution was set to 120,000 (at 200 m/z), mass range of 375-1650 m/z, AGC of 1e6 and maximum injection time was set to 60 msec. MS2 resolution was set to 30,000, quadrupole isolation 1.7 m/z, AGC of 2e5, and maximum injection time of 100 msec.

Data processing. LFQ raw data were processed with MaxQuant v1.6.0.16. The data were searched with the Andromeda search engine against a database containing the mouse (Mus musculus) or human (Homo sapiens) protein sequences (corresponding to sample origin) as downloaded from Uniprot.org, and appended with common laboratory protein contaminants. Enzyme specificity was set to trypsin and up to two missed cleavages were allowed. A fixed modification was set to carbamidomethylation of cysteines and variable modifications were set to oxidation of methionines and deamidation of asparagines and glutamines. Peptide precursor ions were searched with a maximum mass deviation of 4.5 ppm and fragment ions with a maximum mass deviation of 20 ppm. Peptide and protein identifications were filtered at a false discovery rate (FDR) of 1% using the decoy database strategy (MaxQuant's “Revert” module). The minimal peptide length was 7 amino-acids, and the minimum Andromeda score for modified peptides was 40. Peptide identifications were propagated across samples using the match-between-runs option checked. Searches were performed with the label-free quantification option selected. The quantitative comparisons were calculated using Perseus v1.6.0.7. Decoy hits were filtered out and only proteins that had at least 50% valid values in at least one experimental group were kept. Missing data were replaced using imputation, assuming normal distribution with a downshift of 1.8 standard deviations and a width of 0.3 of the original ratio distributions.

PRM data was processed with the Skyline algorithm. Extracted ion chromatograms for all relevant peptide precursors and fragments were imported and manually curated for removing interfering signals and determining peak boundaries. The signal from SIL peptides was used to validate assignment of peak identity of the corresponding light (native) peptides. Peak assignment validation of native peptides without a corresponding SIL peptide was done by constructing a peptide identification library in Skyline using the above LFQ analysis results and comparing to PRM data. Peptide assignments of PRM fragment clusters which did not have a high confidence match to the relevant peptide in the library (dotp score<0.80) in any sample were filtered out. Total fragment area was exported from Skyline to Microsoft Excel, normalized to total ion current and log-transformed for statistical analysis.

Proteomics data analysis. Mass spectrometry intensities were log-transformed. Principal component analysis (PCA) was carried out based on log-transformed intensity. Permutational multivariate analysis of variance (PERMANOVA) was performed for pairwise comparisons on a distance matrix between group levels with corrections for multiple testing using the RVAideMemoire R package (available at the CRAN-R website under “packages”/“RVAideMemoire”). We generated clustered heatmaps using hierarchical clustering on Euclidean distance with average linkage. Differential protein expression was tested applying Mann-Whitney U test (for independent comparison) or Wilcoxon signed-rank test (for paired comparison) with FDR correction, and Log 2 fold changes were calculated. We tested for non-random behavior of the proteomic AMP signature against the background of the entire proteomic landscape by applying pre-ranked protein set enrichment analysis using the “fast gene set enrichment analysis” method. Ranks were calculated as signed fold change×−log 10 p-value. Venn diagrams were created to identify intersecting protein signatures. Kruskal—Wallis one-way analysis of variance (for independent comparison) or one-way repeated measures ANOVA on rank transformed values (for paired comparison) was used for the proteomic comparison of more than 2 groups.

The mass spectrometry proteomics data have been deposited to the ProteomeXchange Consortium via the PRIDE partner repository with the dataset identifier PXD024793.

Microbiome Sequencing and Analysis

DNA purification. DNA from intestinal mucosa (ileum, distal colon) or stool was extracted and purified using a Purelink Microbiome DNA purification kit (Invitrogen, Thermo-Fisher Scientific, Waltham, MA).

Sequencing of 16S rRNA gene and analysis. For 16S amplicon sequencing, PCR amplification was performed for the 16S rRNA gene, followed by 500-bp paired-end sequencing (Illumina MiSeq, San Diego, CA). Amplicons spanning variable region 4 (V4) of the 16S rRNA gene were formed by using the following barcoded primers: Fwd 515F, AATGATACGGCGACCACCGAGATCTACACTATGGTAATTGTGTGCCAGCMGCCGCG GTAA (SEQ ID NO: 1); Rev 806R, CAAGCAGAAGACGGCATA CGAGATXXXXXXXXXXXXAGTCAGTCAGCCGGACTACHVGGGTWTCTAAT; in which X represents a barcode base. W=A or T; M=A or C; H=A or C or T (SEQ ID NO: 2). Illumina's bcl2fastq script was used to generate the fastq files. Matched paired-end FASTQ files were processed using the Qiime2 software (q2cli version 2019.7.0). Demultiplexing was performed according to sample-specific barcodes. Bases of poor quality were trimmed. The sequences were denoised binned to amplicon sequencing variants (ASVs) employing the dada2 plugin for Qiime2. Taxonomic assignment was performed using the naive Bayes feature classifier and the Greengenes 13_8 database.

Shotgun metagenomics sequencing and processing. For shotgun sequencing, Illumina libraries were prepared using a Nextera DNA Sample Prep kit (Rumina, FC-121-1031), according to the manufacturer's protocol, and sequenced on the Illumina NextSeq platform with a read length of 80 bp. Illumina's bcl2fastq script was implemented to generate the fastq files. Reads were QC trimmed using Trimmomatic choosing the parameters PE-threads 10-phred33-validatePairs ILLUMINACLIP:TruSeq3-PE.fa:2:30:10 LEADING:3 TRAILING:3 MINLEN:50. KneadData (v0.7.2) was used with default parameters to remove host reads using mm9 as the reference. The cleared fastq files were subsampled using Seqtk (1.2-r94) (www(dot)github(dot)com/lh3/seqtk). Taxonomic assignment of bacterial DNA was carried out relying on exact alignment of k-mers with Kraken2 (v1.1.1) against the Genome Taxonomy Database (see Ecogenomic website www(dot)gtdb(dot)ecogenomic(dot)org/). To improve the accuracy of genus and species level classification, Bayesian re-estimation of bacterial abundance with Bracken (v1.0) was employed. The counts for both genus and species levels were separated, and the count tables to the lowest sequencing depth on an experiment-wise basis (˜500 k) were subsampled. Additionally, bacteria which failed to reach a total abundance of at least 0.01 were removed. Functional annotation was implemented using protein alignment with DIAMOND, thereby only the first hit was considered, an e value <0.0001 was accepted. We then used EMPANADA for sample-specific assignment of gene families to pathways.

Numerical ecology analysis. The microbial community ecology was analyzed with R (v3.6.3), mainly relying on the vegan and ade4 packages. The ASV abundance tables were normalized by subsampling the libraries to the lowest sequencing depth in the respective experiment and either by total sums scaling normalization (TSS). For ordination, principal component analysis applying center log-ratio transformation (CLR) was employed. Differential abundance testing of individual bacterial genera or species was carried out using Mann—Whitney U test (for independent comparison between two groups) or Wilcoxon signed-rank test (for paired comparison between two groups), and log 2 fold changes were calculated. P-values were adjusted for the FDR.

RNA-Sequencing and Analysis

RNA purification. Total RNA from intestinal mucosal samples (terminal ileum, distal colon) were extracted and purified using RNAeasy kit (QIAGEN, 74104, Germantown, MD) according to the manufacturer's instructions.

RNA-sequencing. Ribosomal RNA was selectively depleted by RnaseH (New England Biolabs, M0297) according to a modified version of a published method (7). Specifically, a pool of 50 bp DNA oligos (25 nM, IDT) that is complementary to murine rRNA18S and 28S, was resuspended in 75 ml of 10 mM Tris pH 8.0. Total RNA (1000 ng in 10 ml H₂O) was mixed with an equal amount of rRNA oligo pool. The RNA was added to 2 μl diluted oligo pool and 3 μl 5×rRNA hybridization buffer (0.5M Tris-HCl, 1M NaCl, titrated with HCl to pH 7.4). Samples were incubated at 95° C. for 2 min, then the temperature was slowly reduced (_0.1_C/s) to 37° C. RNaseH enzyme mix (2 μl of 10 U RNaseH, 2 μL 10×RNaseH buffer, 1 mL H₂O, total 5 μl mix) was prepared 5 min before the end of the hybridization and preheated to 37° C. The enzyme mix was added to the samples when they reached 37° C. and they were incubated at this temperature for 30 min. Samples were purified with 2.2×SPRI beads (Ampure XP, Beckmann Coulter, Indianapolis, IN) according to the manufacturers' instructions. Residual oligos were removed with DNase treatment (ThermoFisher Scientific, AM2238) by incubation with 51.11 DNase reaction mix (1 μl Trubo DNase, 2.5 μl Turbo DNase 10× buffer, 1.5 μl H₂O) that was incubated at 37° C. for 30 min. Samples were again purified with 2.2×SPRI beads and suspended in 3.6 μl priming mix (0.3 ml random primers of New England Biolab, E7420, 3.3 μl H₂O, Ipswich, MA). Samples were subsequently primed at 65° C. for 5 min. Samples were then transferred to ice and 2 μl of the first strand mix was added (1 μl 5× first strand buffer, NEB E7420; 0.125 μl RNase inhibitor, NEB E7420; 0.25 μl Prot® Script II reverse transcriptase, NEB E7420; and 0.625 μl of 0.2 μg/ml Actinomycin D, Sigma, A1410). The first strand synthesis and all subsequent library preparation steps were performed using NEBNext Ultra Directional RNA Library Prep Kit for Illumina (NEB, E7420) according to the manufacturers' instructions (all reaction volumes reduced to a quarter). Libraries that passed quality control were loaded with a concentration of 2 pM on 75 cycle high output flow cells (Illumina, FC-404-2005) and sequenced on a NextSeq 500 (I lumina) with the following cycle distribution: 8 bp index 1, 8 bp index 2, 75 bp read 1.

RNA-sequencing analysis. Illumina's bcl2fastq script was used to convert the raw files to fastq files. Fastq files were quality filtered using fastp (v0.20.0) with default parameters. Reads were aligned to the murine reference transcriptome with Gencode annotation (GRCm38.p6, Release M24) and quantified gene expression using STAR (v2.7.3a). Differential expression analysis was carried out with the DESeq2 package with default parameters. Gene Ontology analysis was carried out with g:Profiler with default settings. Comparisons of protein expression on the transcriptomic and the proteomic levels were carried out using heatmaps or Venn diagrams.

Proteome-Microbiome Integration Analysis

To assess the association between bacterial (SFB and C. rodentium) abundance and the AMP landscape, the gradient of the respective microbe's relative abundance in the ordination space was incorporated using the vegan function ordisurf with fitting a generalized additive model (GAM). The function ordisurf fits a smooth surface using penalized splines. In addition, Spearman's rank correlation analysis was performed to assess the association between bacterial abundance and individual AMP abundance.

To assess the association between proteome and microbiome on a landscape level, Procrustes rotation of two configurations was used, as available in the vegan package. Euclidean distances were calculated for both data domains. AMPs were used as the target matrix X and bacterial species as the matrix to be rotated Y. The protest function was used to obtain Monte Carlo p-values for testing of rotational agreement significance using 9,999 permutations.

For the identification of pairwise associations between individual proteins and either species or metagenomic functional pathways sparse partial least squares (sPLS) modeling as provided in the mixOmics data integration project was implemented. A particular advantage of sPLS is that it can accommodate numerous noisy and collinear (correlated) variables, and can also simultaneously model several response variables Y. It is efficient in large p, small n scenarios. Variable selection is achieved by introducing LASSO penalization on the pair of loading vectors. Bacterial species or pathways were regressed against the AMP expression levels. Only species with a total abundance of at least 0.01 and a persistence of 25% of the samples were considered. CLR was applied to the response variables. Multilevel decomposition was applied to account for repeated measurements. The model was tuned by M-fold cross-validation with 5 folds and 100 iterations. The mean squared error was estimated, R2 and Q2. An X variable was considered to contribute significantly to the prediction when Q2 h≥(1-0.95²)=0.0975, as recommended by the developers. The cim function was used to illustrate the variable associations identified at a correlation threshold of 0.70 (for murine DSS model) or 0.35 (for human data).

Predictive Modeling of Disease Status Using AMP Proteomic and Microbiome Landscape

A support vector machine (SVM) with a linear kernel implemented in R package e1071 (see CRAN-R project website under “packages” and “e1071) was used to classify the phases of DSS-induced colitis as this classifier is both high performing and parsimonious. Separate classifiers were trained using either AMPs or metagenomic species information as input features. “Leave group out” cross-validation was used to avoid overfitting due to cage effects, whereby samples from one cage were left out in every training iteration. In each step, the prediction was performed on the cage set aside. The performance was evaluated using a confusion matrix with accuracy.

For the prediction of the area under the curve (AUC) of weight loss as a proxy of disease severity in mice challenged with DSS, gradient boosting regression algorithms were used to model both AMP and microbiome features, with the default parameters of sklearn python package (www(dot)scikit-learn(dot)org/stable/) with a single change to the minimal number of leaves to five, in order to avoid cage discriminating splits. An additional model for the microbiome data with a pre-processing step of PCA was built, in order to reduce the dimensionality of the data. In this model the PCA was computed only on the training data and the test sample was projected based on the training data, and then employed gradient boosting regression algorithm. The same leave one cage out cross-validation scheme described above was employed for analysis. The performance was evaluated using Pearson correlation coefficient between predicted and observed weight loss, and mean square error (MSE).

For the classification of human Crohn's disease versus healthy status, sigmoid kernel SVM classifiers were trained on the fecal AMP profile, fecal microbiome composition profile, and combined profiles, using a stratified K-fold cross validation (K=5) to avoid gender bias. Classification performance was summarized by ROC curves.

Human Study Design

The human study was approved by the Schneider Children's Hospital Institutional Review Board (IRB approval number 0722-19-RMC). In total, 54 children were recruited, including 26 pediatric Crohn's disease (CD) patients whose diagnosis were based on standard endoscopic, radiographical and histological criteria, and 28 healthy controls (HC). Participants from these 2 arms were matched by age and gender to avoid both confounding factors. All subjects fulfilled the following inclusion criteria: males and females, aged 6-18. Exclusion criteria included: (i) chronic treatment with any drug upon enrollment for healthy controls; (ii) the use of systemic antibiotics, probiotics or proton pump inhibitors 3 months prior to enrollment; (iii) diagnosed with type 1 or type 2 diabetes; (iv) any chronic disease (other than IBD for the first arm); (v) any psychiatric disorders; (vi) alcohol or substance abuse; (vii) gut-related surgery, including bariatric surgery; (viii) pregnancy, breastfeeding or fertility treatments for female participants; (ix) morbid obesity (BMI>95^{th} percentile for their age and gender). After signing the informed consent, participants enrolled in both arms collected stool samples using the same protocol: a single stool sample was collected at home and then frozen in a home freezer up to 7 days and then brought on site in a cold cooler. Samples were then frozen and stored at −80° C. before protein was extracted for proteomics and DNA was extracted for shotgun metagenomic sequencing, as described above respectively.

### Results

### Example 1: A Proteomics-Metagenomics Pipeline for Identification of the Intestinal AMP Landscape

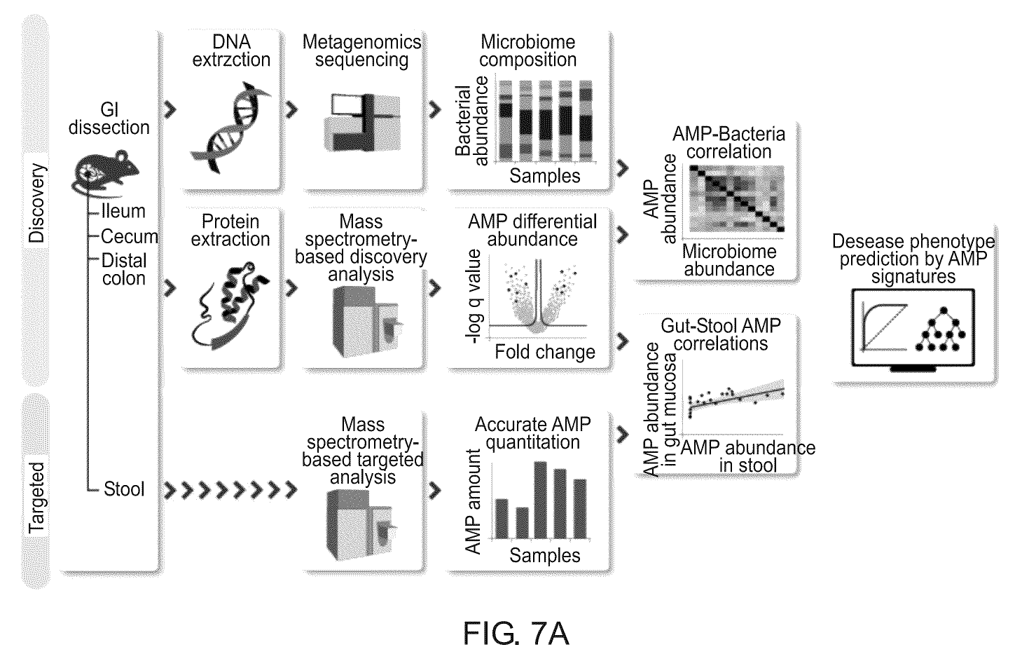

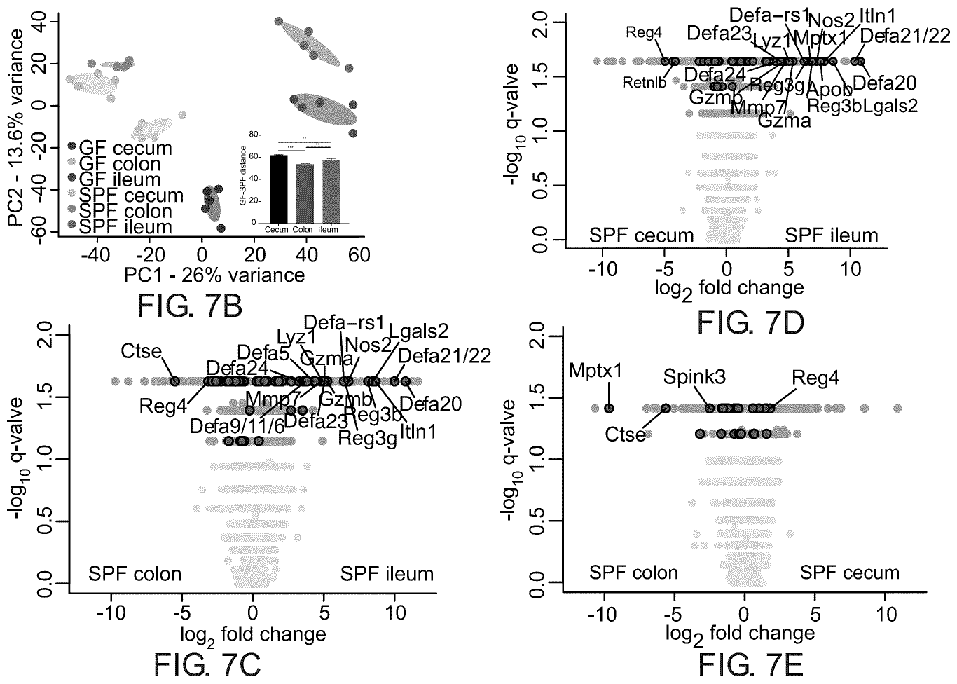

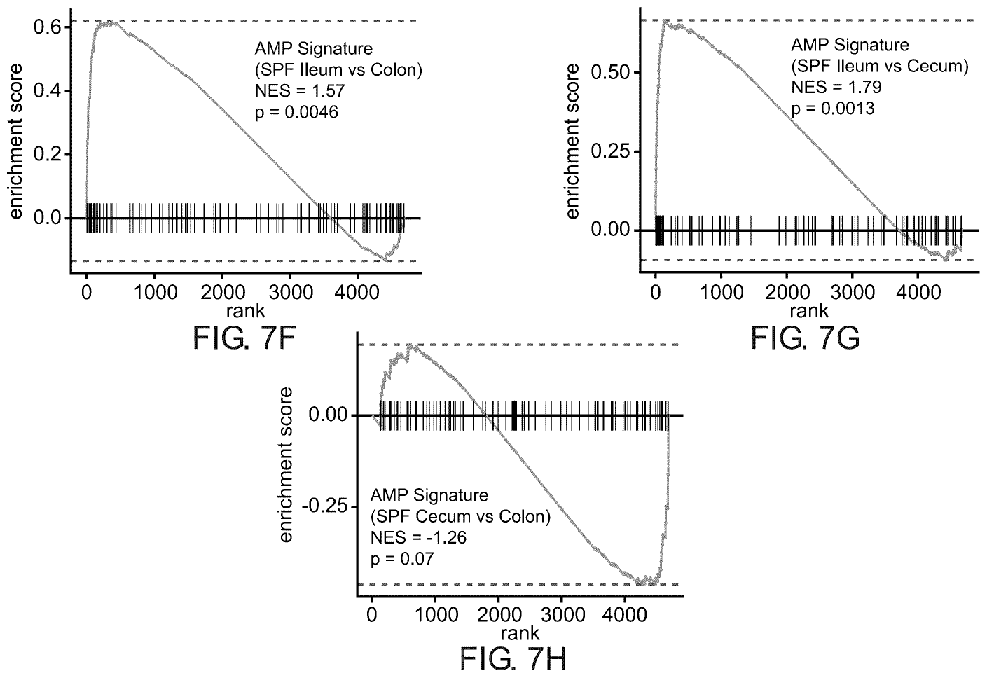

The proteomics-metagenomics pipeline was designed to systematically identify and quantify the intestinal AMP landscape in mice as depicted in FIG. 7A. In short, following harvesting of ileal, cecal and colonic mucosal samples, depletion of proteins associated with membranes and membrane-bound organelles was achieved by mild homogenization of mucosal samples in a detergent-free buffer. The soluble fraction was then trypsin digested and analyzed by label-free quantitative (LFQ) proteomics (‘discovery approach’). To further focus on the AMP signature, quantitative information was obtained on a total of 141 proteins and peptides, including 116 known AMPs whose antimicrobial activities have been documented by previous studies (Table S1), and 25 ‘AMP candidates’ recognized by meeting at least 2 out of the following 4 criteria: A) secreted protein or peptide; B) structurally similar to known AMPs, of the lectin, cathepsin, cystatin, pentraxin, resistin-like, calcium-binding 5100 protein families, or possessing any bacterial ligand-binding domains; C) functionally enhancing the antibacterial defense response, or bearing immunomodulatory activities; D) featuring similar abundance pattern to known AMPs upon commensal colonization, pathogenic infection or intestinal inflammation (Table SlA).

TABLE S1
Annotated AMPs

| | | Secreted | Uniprot |
|---|---|---|---|
| Protein name | Gene name | (yes/no) | accession |
| Angiogenin-4 | Ang4 | yes | Q3TMQ6 |
| Serum amyloid P-component | Apcs | yes | P12246 |
| Apolipoprotein A-I | Apoa1 | yes | Q00623 |
| Apolipoprotein A-II | Apoa2 | yes | P09813 |
| Apolipoprotein B-100 | Apob | yes | E9Q414 |
| Apolipoprotein E | Apoe | yes | P08226 |
| Beta-2-glycoprotein 1/Apolipoprotein H | Apoh/B2gp1 | yes | Q01339 |
| Amyloid-beta A4 protein | App | no | P12023 |
| Arginase-1 | Arg1 | no | Q61176 |
| Beta-2-microglobulin | B2m | yes | P01887 |
| 3-hydroxybutyrate dehydrogenase type 2 | Bdh2 | no | Q8JZV9 |
| BPI fold-containing family A member 2 | Bpifa2 | yes | P07743 |
| Cathelicidin antimicrobial peptide | Camp | yes | P51437 |
| CD5 antigen-like | Cd51 | yes | Q9QWK4 |
| Chromogranin-A | Chga | yes | P26339 |
| Secretogranin-1 | Chgb | yes | P16014 |
| Chitinase-3-like protein 1 | Chi3l1 | yes | Q61362 |
| Acidic mammalian chitinase | Chia | yes | Q91XA9 |
| Cystatin-C | Cst3 | yes | P21460 |
| Cathepsin B | Ctsb | yes | Q62426 |
| Cathepsin G | Ctsg | yes | P28293 |
| Alpha-defensin-related sequence 1 | Defa-rs1 | yes | P17533 |
| Alpha-defensin | Defa11 | yes | P50709 |
| Alpha-defensin | Defa24 | yes | Q5G865 |
| Alpha-defensin | Defa15 | yes | P50713 |
| Alpha-defensin | Defa9 | yes | P50707 |
| Alpha-defensin | Defa6 | yes | P50704 |
| Alpha-defensin | Defa17/1 | yes | Q64016 |
| Alpha-defensin | Defa1 | yes | P11477 |
| Alpha-defensin | Defa20 | yes | Q45VN2 |
| Alpha-defensin | Defa21 | yes | Q8C1P2 |
| Alpha-defensin | Defa22 | yes | Q8C1N8 |
| Alpha-defensin | Defa8 | yes | P50706 |
| Alpha-defensin | Defa16 | yes | P50714 |
| Alpha-defensin | Defa7 | yes | P50705 |
| Alpha-defensin | Defa3 | yes | P28310 |
| Alpha-defensin | Defa2 | yes | P28309 |
| Alpha-defensin | Defa13 | yes | P50711 |
| Alpha-defensin | Defa5 | yes | P28312 |
| Deleted in malignant brain tumors 1 protein | Dmbt1 | yes | Q60997 |
| NAD(P)H oxidase (H(2)O(2)-forming) | Duox2 | no | A2AQ99 |
| Dual oxidase maturation factor 2 | Duoxa2 | no | Q9D311 |
| Eosinophil cationic protein 1 | Ear1 | yes | P97426 |
| Eosinophil cationic protein 2 | Ear2 | yes | P97425 |
| Neutrophil elastase | Elane | yes | Q3UP87 |
| Engulfment and cell motility protein 1 | Elmo1 | no | Q8BPU7 |
| Eosinophil peroxidase | Epx | no | P49290 |
| Protein FAM49B | Fam49b | no | Q921M7 |
| Ficolin-1 | Fcn1 | yes | O70165 |
| Fibronectin | Fn1 | yes | P11276 |
| Pancreatic secretory granule membrane | Gp2 | yes | Q9D733 |
| major glycoprotein GP2 | | | |
| Progranulin | Grn | yes | P28798 |
| Gasdermin-A | Gsdma | yes | Q9EST1 |
| Gasdermin-C2 | Gsdmc2 | yes | Q2KHK6 |
| Gasdermin-D | Gsdmdc1 | yes | Q9D8T2 |
| Gelsolin | Gsn | yes | P13020 |
| Granzyme A | Gzma | yes | P11032 |
| Granzyme B(G, H) | Gzmb | no | P04187 |
| Hepatocyte growth factor activator | Hgfac | yes | Q9R098 |
| High mobility group protein B1 | Hmgb1 | yes | P63158 |
| High mobility group protein B2 | Hmgb2 | yes | P30681 |
| High mobility group protein B3 | Hmgb3 | no | O54879 |
| Histidine-rich glycoprotein | Hrg | yes | Q9ESB3 |
| Intestinal Alkaline Phosphatase | Iap | yes | P24822 |
| Indoleamine 2,3-dioxygenase 1 | Ido1 | no | P28776 |
| Ubiquitin-like protein ISG15 | Isg15 | yes | Q64339 |
| Intelectin-1a | Itln1 | yes | O88310 |
| Lipopolysaccharide-binding protein | Lbp | yes | Q61805 |
| Neutrophil gelatinase-associated lipocalin | Lcn2 | yes | P11672 |
| Plastin-2 | Lcp1 | no | Q61233 |
| Galectin-1 | Lgals1 | yes | P16045 |
| Galectin-2 | Lgals2 | yes | Q9CQW5 |
| Galectin-3 | Lgals3 | yes | P16110 |
| Galectin-4 | Lgals4 | yes | Q8K419 |
| Galectin-9 | Lgals9 | yes | O08573 |
| Lactotransferrin | Ltf | yes | P08071 |
| Lumican | Lum | yes | P51885 |
| Ly6/PLAUR domain-containing protein 8 | Lypd8 | yes | Q9D7S0 |
| Lysozyme C-1 | Lyz1 | yes | P17897 |
| Lysozyme C-2 | Lyz2 | yes | P08905 |
| Mannose-binding protein A | Mbl1 | yes | P39039 |
| Mannose-binding protein C | Mbl2 | yes | P41317 |
| Macrophage migration inhibitory factor | Mif | yes | P34884 |
| Stromelysin-1; Stromelysin-2 | Mmp3; Mmp10 | yes | P28862 |
| Matrilysin | Mmp7 | yes | Q10738 |
| Myeloperoxidase | Mpo | no | P11247 |
| Neutrophilic granule protein | Ngp | yes | O08692 |
| Nitric oxide synthase, inducible | Nos2 | no | P29477 |
| Alpha-1-acid glycoprotein 1 | Orm1 | yes | Q60590 |
| Alpha-1-acid glycoprotein 2 | Orm2 | yes | P07361 |
| Peptidoglycan recognition protein 1 | Pglyrp1 | yes | O88593 |
| N-acetylmuramoyl-L-alanine amidase | Pglyrp2 | yes | Q8VCS0 |
| Group 10 secretory phospholipase A2 | Pla2g10 | yes | Q9QXX3 |
| Phospholipase A2 | Pla2g1b | yes | Q9Z0Y2 |
| Phospholipase A2, membrane associated | Pla2g2a | yes | P31482 |
| Platelet-activating factor acetylhydrolase | Pla2g7 | yes | Q60963 |
| Phospholipid transfer protein | Pltp | yes | P55065 |
| Serum paraoxonase/arylesterase 1 | Pon1 | yes | P52430 |
| Serum paraoxonase/arylesterase 2 | Pon2 | no | Q62086 |
| Serum paraoxonase/lactonase 3 | Pon3 | yes | Q62087 |
| Bone marrow proteoglycan | Prg2 | no | Q61878 |
| Proteoglycan 4 | Prg4 | yes | Q9JM99 |
| Myeloblastin | Prtn3 | yes | Q61096 |
| Prosaposin | Psap | yes | Q61207 |
| Regenerating islet-derived protein 3-alpha | Reg3a | yes | O09037 |
| Regenerating islet-derived protein 3-beta | Reg3b | yes | P35230 |
| Regenerating islet-derived protein 3-gamma | Reg3g | yes | O09049 |
| Regenerating islet-derived protein 4 | Reg4 | yes | Q9D8G5 |
| Resistin-like beta | Retnlb | yes | Q99P86 |
| Ribonuclease pancreatic | Rnase1 | yes | P00683 |
| Ribonuclease4 | Rnase4 | yes | Q9JJH1 |
| Protein S100-A11 | S100a11 | yes | P50543 |
| Protein S100-A8 | S100a8 | yes | P27005 |
| Protein S100-A9 | S100a9 | yes | P31725 |
| Serum amyloid A-1 protein | Saa1 | yes | P05366 |
| Serum amyloid A-2 protein | Saa2 | yes | P05367 |
| Serum amyloid A-4 protein | Saa4 | yes | P31532 |
| Selenoprotein P | Sepp1 | yes | P70274 |
| Alpha-1-antitrypsin 1 | Serpina1 | yes | P07758 |
| Alpha-1-antitrypsin 1 | Serpina1 | yes | P22599 |
| Alpha-1-antitrypsin 1 | Serpina1 | yes | Q00896 |
| Alpha-1-antitrypsin 1 | Serpina1 | yes | Q00897 |
| Alpha-1-antitrypsin 1 | Serpina1 | yes | Q00898 |
| Leukocyte elastase inhibitor A | Serpinb1a | yes | Q9D154 |
| Antileukoproteinase | Slpi | yes | P97430 |
| Serine protease inhibitor Kazal-type 1 | Spink3 | yes | P09036 |
| Trefoil factor 2 | Tff2 | yes | Q03404 |
| Trefoil factor 3 | Tff3 | yes | Q62395 |
| Thrombospondin-1 | Thbs1 | yes | Q80YQ1 |
| Vitronectin | Vtn | yes | P29788 |
| Zymogen granule membrane protein 16 | Zg16 | yes | Q8K0C5 |

TABLE S1A
Candidate AMPs

| | Gene | UniProt | SEQ |
|---|---|---|---|
| Protein name | name | Access No. | ID No |
| Angiotensin-converting enzyme 2 | Ace | P09470 | 17 |
| Adiponectin | Adipoq | Q60994 | 27 |
| Neutral ceramidase | Asah2 | Q9JHE3 | 18 |
| Zinc-alpha-2-glycoprotein | Azgp1 | Q64726 | 26 |
| CD177 antigen | Cd177 | Q8R2S8 | 21 |
| Chitinase-like protein 3 | Chil3 | O35744 | 15 |
| Tetranectin | Clec3b | P43025 | 9 |
| Clusterin | Clu | Q06890 | 6 |
| Cystatin-A; Cystatin-A, N- | Csta | P56567 | 12 |
| terminally processed | | | |
| Cystatin-B | Cstb | Q62426 | 10 |
| Cathepsin D | Ctsd | P18242 | 3 |
| Cathepsin E | Ctse | P70269 | 23 |
| Cathepsin L1; Cathepsin L1 heavy | Ctsl | P06797 | 4 |
| chain; Cathepsin L1 light chain | | | |
| Hyaluronan-binding protein 2 | Habp2 | Q8K0D2 | 16 |
| Integrin alpha-M | Itgam | P05555 | 5 |
| Integrin beta-2 | Itgb2 | P11835 | 11 |
| Mesencephalic astrocyte-derived | Manf | Q9CXI5 | 13 |
| neurotrophic factor | | | |
| Matrix metalloproteinase-9 | Mmp9 | P41245 | 24 |
| Mucosal pentraxin | Mptx1 | Q8R1M8 | 22 |
| Cytosolic phospholipase A2 | Pla2g4c | Q64GA5 | 19 |
| gamma | | | |
| Group XV phospholipase A2 | Pla2g15 | Q8VEB4 | 14 |
| Lithostathine-1 | Reg1 | P43137 | 7 |
| Lithostathine-2 | Reg2 | Q08731 | 8 |
| Resistin-like gamma | Retnlg | Q8K426 | 20 |
| Protein S100-A13 | S100a13 | P97325 | 25 |

In complementing the discovery approach and the inherent technical limitations of stool proteomics, these known AMPs and AMP candidates were then monitored using a parallel fecal targeted AMP-focused proteomic approach (‘targeted approach’) enabling increased sensitivity and high confidence in AMP identification and quantification in the complex stool and GI mucosal sample matrix. Specifically, 148 tryptic peptides corresponding to 68 known or putative AMPs were measured in stool using a custom-designed parallel reaction monitoring (PRM) pipeline (Table S2)(sequences not shown).

TABLE S2

| | | | | Available |
|---|---|---|---|---|
| | | | | stable |
| | | | | isotope |
| Uniprot | | | | labeled |
| protein | Protein | Gene | | (SIL) |
| accession | name | name | Protein descripton | peptide |
| Q60590 | A1AG1 | Orm1 | Alpha-1-acid glycoprotein 1 | No |
| Q60590 | A1AG1 | Orm1 | Alpha-1-acid glycoprotein 1 | No |
| Q60590 | A1AG1 | Orm1 | Alpha-1-acid glycoprotein 1 | Yes |
| Q3TMQ6 | ANG4 | Ang4 | Angiogenin-4 | Yes |
| Q3TMQ6 | ANG4 | Ang4 | Angiogenin-4 | Yes |
| Q3TMQ6 | ANG4 | Ang4 | Angiogenin-4 | Yes |
| Q9JHE3 | ASAH2 | Asah2 | Neutral ceramidase | No |
| Q9JHE3 | ASAH2 | Asah2 | Neutral ceramidase | No |
| Q9JHE3 | ASAH2 | Asah2 | Neutral ceramidase | No |
| P07743 | BPIA2 | Bpifa2 | BPI fold-containing family A | No |
| | | | member 2 | |
| P07743 | BPIA2 | Bpifa2 | BPI fold-containing family A | No |
| | | | member 2 | |
| P07743 | BPIA2 | Bpifa2 | BPI fold-containing family A | No |
| | | | member 2 | |
| P51437 | CAMP | Camp | Cathelicidin antimicrobialptide | No |
| P51437 | CAMP | Camp | Cathelicidin antimicrobialptide | No |
| P51437 | CAMP | Camp | Cathelicidin antimicrobialptide | No |
| Q91XA9 | CHIA | Chia | Acidic mammalian chitinase | No |
| O35744 | CHIL3 | Chil3 | Chitinase-like protein 3 | No |
| O35744 | CHIL3 | Chil3 | Chitinase-like protein 3 | No |
| O35744 | CHIL3 | Chil3 | Chitinase-like protein 3 | No |
| P56567 | CYTA | Csta | Cystatin-A | No |
| P56567 | CYTA | Csta | Cystatin-A | No |
| Q62426 | CYTB | Cstb | Cystatin-B | No |
| Q62426 | CYTB | Cstb | Cystatin-B | Yes |
| P21460 | CYTC | Cst3 | Cystatin-C | No |
| P21460 | CYTC | Cst3 | Cystatin-C | Yes |
| P11477 | DEFA1 | Defa1 | Alpha-defensin 1 | No |
| P28309 | DEFA2 | Defa2 | Alpha-defensin 2 | No |
| P28310 | DEFA3 | Defa3 | Alpha-defensin 3 | No |
| E9QPZ2 | DEFA30 | Defa30 | Alpha-defensin 30 | Yes |
| P28312 | DEFA5 | Defa5 | Alpha-defensin 5 | No |
| P50704 | DEFA6 | Defa6 | Alpha-defensin 6/12 | No |
| Q45VN2 | DFA20 | Defa20 | Alpha-defensin 20 | No |
| Q45VN2 | DFA20 | Defa20 | Alpha-defensin 20 | Yes |
| Q8C1P2 | DFA21 | Defa21 | Alpha-defensin 21 | No |
| Q8C1P2 | DFA21 | Defa21 | Alpha-defensin 21 | No |
| P17533 | DFAR1 | Defars1 | Alpha-defensin-related | No |
| | | | sequence 1 | |
| P17533 | DFAR1 | Defars1 | Alpha-defensin-related | No |
| | | | sequence 1 | |
| Q8R218 | DFB10 | Defb10 | Beta-defensin 10 | Yes |
| Q30KN7 | DFB26 | Defb26 | Beta-defensin 26 | Yes |
| Q60997 | DMBT1 | Dmbt1 | Deleted in malignant brain | No |
| | | | tumors 1 protein | |
| Q60997 | DMBT1 | Dmbt1 | Deleted in malignant brain | No |
| | | | tumors 1 protein | |
| Q60997 | DMBT1 | Dmbt1 | Deleted in malignant brain | No |
| | | | tumors 1 protein | |
| P97425 | ECP2 | Ear2 | Eosinophil cationic protein 2 | No |
| Q3UP87 | ELNE | Elane | Neutrophil elastase | No |
| Q3UP87 | ELNE | Elane | Neutrophil elastase | No |
| P11032 | GRAA | Gzma | Granzyme A | No |
| P11032 | GRAA | Gzma | Granzyme A | No |
| P11032 | GRAA | Gzma | Granzyme A | No |
| P04187 | GRAB | Gzmb | Granzyme B(G, H) | No |
| P04187 | GRAB | Gzmb | Granzyme B(G, H) | No |
| P04187 | GRAB | Gzmb | Granzyme B(G, H) | No |
| Q2KHK6 | GSDC2 | Gsdmc2 | Gasdermin-C2 | No |
| Q2KHK6 | GSDC2 | Gsdmc2 | Gasdermin-C2 | No |
| Q2KHK6 | GSDC2 | Gsdmc2 | Gasdermin-C2 | No |
| P09036 | ISK1 | Spink1 | Serine | No |
| | | | protease inhibitor | |
| | | | Kazal-type 1 | |
| P09036 | ISK1 | Spink1 | Serine protease inhibitor | No |
| | | | Kazal-type 1 | |
| P09036 | ISK1 | Spink1 | Serine protease inhibitor | No |
| | | | Kazal-type 1 | |
| O88310 | ITL1A | Itln1 | Intelectin-1a | No |
| O88310 | ITL1A | Itln1 | Intelectin-1a | No |
| O88310 | ITL1A | Itln1 | Intelectin-1a | No |
| Q9CQW5 | LEG2 | Lgals2 | Galectin-2 | No |
| Q9CQW5 | LEG2 | Lgals2 | Galectin-2 | No |
| Q9CQW5 | LEG2 | Lgals2 | Galectin-2 | No |
| P43137 | LIT1 | Reg1 | Lithostathine-1 | No |
| P43137 | LIT1 | Reg1 | Lithostathine-1 | No |
| P43137 | LIT1 | Reg1 | Lithostathine-1 | No |
| Q08731 | LIT2 | Reg2 | Lithostathine-2 | No |
| P17897 | LYZ1 | Lyz1 | Lysozyme C-1 | No |
| P17897 | LYZ1 | Lyz1 | Lysozyme C-1 | Yes |
| P08905 | LYZ2 | Lyz2 | Lysozyme C-2 | No |
| P08905 | LYZ2 | Lyz2 | Lysozyme C-2 | No |
| Q10738 | MMP7 | Mmp7 | Matrilysin | No |
| Q10738 | MMP7 | Mmp7 | Matrilysin | No |
| Q8R1M8 | MPTX | Mptx1 | Mucosalntraxin | No |
| Q8R1M8 | MPTX | Mptx1 | Mucosalntraxin | No |
| Q8R1M8 | MPTX | Mptx1 | Mucosalntraxin | No |
| P11672 | NGAL | Lcn2 | Neutrophil gelatinase- | No |
| | | | associated lipocalin | |
| P11672 | NGAL | Lcn2 | Neutrophil gelatinase- | No |
| | | | associated lipocalin | |
| P11672 | NGAL | Lcn2 | Neutrophil gelatinase- | No |
| | | | associated lipocalin | |
| O08692 | NGP | Ngp | Neutrophilic granule protein | No |
| O08692 | NGP | Ngp | Neutrophilic granule protein | No |
| O08692 | NGP | Ngp | Neutrophilic granule protein | No |
| P29477 | NOS2 | Nos2 | Nitric oxide synthase, | No |
| | | | inducible | |
| P29477 | NOS2 | Nos2 | Nitric oxide synthase, | No |
| | | | inducible | |
| P29477 | NOS2 | Nos2 | Nitric oxide synthase, | No |
| | | | inducible | |
| Q9ZOY2 | PA21B | Pla2g1b | Phospholipase A2 | No |
| Q9ZOY2 | PA21B | Pla2g1b | Phospholipase A2 | No |
| Q9ZOY2 | PA21B | Pla2g1b | Phospholipase A2 | No |
| Q64GA5 | PA24C | Pla2g4c | Cytosolic phospholipase A2 | No |
| | | | gamma | |
| Q64GA5 | PA24C | Pla2g4c | Cytosolic phospholipase A2 | No |
| | | | gamma | |
| P31482 | PA2GA | Pla2g2a | Phospholipase A2, membrane | No |
| | | | associated | |
| Q8VEB4 | PAG15 | Pla2g15 | Group XV phospholipase A2 | No |
| Q8VEB4 | PAG15 | Pla2g15 | Group XV phospholipase A2 | No |
| P49290 | PERE | Epx | Eosinophilroxidase | No |
| P11247 | PERM | Mpo | Myeloperoxidase | No |
| P11247 | PERM | Mpo | Myeloperoxidase | No |
| P11247 | PERM | Mpo | Myeloperoxidase | No |
| O88593 | PGRP1 | Pglyrp1 | Peptidoglycan recognition | Yes |
| | | | protein 1 | |
| O88593 | PGRP1 | Pglyrp1 | Peptidoglycan recognition | No |
| | | | protein 1 | |
| O88593 | PGRP1 | Pglyrp1 | Peptidoglycan recognition | No |
| | | | protein 1 | |
| Q62086 | PON2 | Pon2 | Serum | No |
| | | | paraoxonase/arylesterase 2 | |
| Q62086 | PON2 | Pon2 | Serum | No |
| | | | paraoxonase/arylesterase 2 | |
| Q62086 | PON2 | Pon2 | Serum | No |
| | | | paraoxonase/arylesterase 2 | |
| Q62087 | PON3 | Pon3 | Serum paraoxonase/lactonase 3 | No |
| Q62087 | PON3 | Pon3 | Serum paraoxonase/lactonase 3 | No |
| Q62087 | PON3 | Pon3 | Serum paraoxonase/lactonase 3 | No |
| Q61878 | PRG2 | Prg2 | Bone marrow proteoglycan | No |
| Q61878 | PRG2 | Prg2 | Bone marrow proteoglycan | No |
| O09037 | REG3A | Reg3a | Regenerating islet-derived | No |
| | | | protein 3-alpha | |
| O09037 | REG3A | Reg3a | Regenerating islet-derived | No |
| | | | protein 3-alpha | |
| O09037 | REG3A | Reg3a | Regenerating islet-derived | No |
| | | | protein 3-alpha | |
| P35230 | REG3B | Reg3b | Regenerating islet-derived | No |
| | | | protein 3-beta | |
| P35230 | REG3B | Reg3b | Regenerating islet-derived | No |
| | | | protein 3-beta | |
| P35230 | REG3B | Reg3b | Regenerating islet-derived | No |
| | | | protein 3-beta | |
| O09049 | REG3G | Reg3g | Regenerating islet-derived | No |
| | | | protein 3-gamma | |
| O09049 | REG3G | Reg3g | Regenerating islet-derived | Yes |
| | | | protein 3-gamma | |
| O09049 | REG3G | Reg3g | Regenerating islet-derived | No |
| | | | protein 3-gamma | |
| Q9D8G5 | REG4 | Reg4 | Regenerating islet-derived | No |
| | | | protein 4 | |
| Q9D8G5 | REG4 | Reg4 | Regenerating islet-derived | No |
| | | | protein 4 | |
| Q9D8G5 | REG4 | Reg4 | Regenerating islet-derived | No |
| | | | protein 4 | |
| Q9EP95 | RETNA | Retnla | Resistin-like alpha | No |
| Q99P86 | RETNB | Retnlb | Resistin-like beta | No |
| Q99P86 | RETNB | Retnlb | Resistin-like beta | No |
| Q8K426 | RETNG | Retnlg | Resistin-like gamma | No |
| P00683 | RNAS1 | Rnase1 | Ribonuclease pancreatic | No |
| Q9JJH1 | RNAS4 | Rnase4 | Ribonuclease 4 | No |
| P27005 | S10A8 | S100a8 | Protein S100-A8 | No |
| P27005 | S10A8 | S100a8 | Protein S100-A8 | No |
| P31725 | S10A9 | S100a9 | Protein S100-A9 | Yes |
| P31725 | S10A9 | S100a9 | Protein S100-A9 | No |
| P31725 | S10A9 | S100a9 | Protein S100-A9 | Yes |
| P50543 | S10AB | S100a11 | Protein S100-A11 | Yes |
| P50543 | S10AB | S100a11 | Protein S100-A11 | Yes |
| P50543 | S10AB | S100a11 | Protein S100-A11 | No |
| P97352 | S10AD | S100a13 | Protein S100-A13 | Yes |
| P97352 | S10AD | S100a13 | Protein S100-A13 | Yes |
| P05366 | SAA1 | Saa1 | Serum amyloid A-1 protein | Yes |
| P05366 | SAA1 | Saa1 | Serum amyloid A-1 protein | Yes |
| P05367 | SAA2 | Saa2 | Serum amyloid A-2 protein | No |
| P12246 | SAMP | Apcs | Serum amyloid P-component | No |
| P97430 | SLPI | Slpi | Antileukoproteinase | No |
| P97430 | SLPI | Slpi | Antileukoproteinase | No |
| P97430 | SLPI | Slpi | Antileukoproteinase | No |
| P43025 | TETN | Clec3b | Tetranectin | No |
| P43025 | TETN | Clec3b | Tetranectin | No |
| P08071 | TRFL | Ltf | Lactotransferrin | No |
| P08071 | TRFL | Ltf | Lactotransferrin | No |
| P08071 | TRFL | Ltf | Lactotransferrin | No |

Stable isotope-labeled (SIL) peptides were synthesized for a subset of 21 of these peptides, spiked into the stool samples, and monitored along with their unlabeled native counterparts in the same experiment, thus serving as internal standards for validation of the respective AMP identity. To further study the inter-relationships between the AMP signature and intestinal microbiome configuration, 16S rRNA gene or shotgun metagenomics sequencing of the same respective samples were performed, as described below.

The AMP landscape along the murine gastrointestinal tract follows a region-specific configuration. To begin with, the intestinal proteomic landscape of 8-week-old C57BL6 mice, kept in germ-free (GF) versus specific pathogen-free (SPF) conditions, sampled in the ileum, cecum and colon was characterized. A total of 4692 proteins were detected by LFQ (discovery) proteomics from 59911 unique peptides. Principal component analysis (PCA) showed that the global proteomic landscape varied by GI region and colonization status, with the differences between GF and SPF mice being more distinct in the cecal and ileal mucosa than in the colonic mucosa (FIG. 7B, Table S3).

TABLE S3

| | Pairwise comparisons | | | | |
|---|---|---|---|---|---|
| | using permutation | | | | |
| | MANOVAs on a | | | | |
| | distance matrix (fdr) | Day 0 | Day 3 | Day 5 | Day 16 |
| Figure | Day 3 | 0.0011 | — | — | — |
| 5D | Day 5 | 0.0011 | 0.0011 | — | — |
| | Day 16 | 0.0011 | 0.0011 | 0.0011 | — |
| | Day 31 | 0.0011 | 0.0011 | 0.0011 | 0.105 |
| | Pairwise comparisons | | | | |
| | using permutation | | | | |
| | MANOVAs on a | | | | |
| | distance matrix (fdr) | Day 0 | Day 3 | Day 5 | Day 16 |
| Not | Day 3 | 0.0011 | — | — | — |
| Shown | Day 5 | 0.0011 | 0.0023 | — | — |
| | Day 16 | 0.0011 | 0.0011 | 0.0011 | — |
| | Day 31 | 0.0011 | 0.0011 | 0.0011 | 0.0011 |
| | Pairwise comparisons | | | | |
| | using permutation | | | | |
| | MANOVAs on a | | | | |
| | distance matrix (fdr) | Day 0 | Day 3 | Day 5 | Day 16 |
| Not | Day 3 | 0.001 | — | — | — |
| Shown | Day 5 | 0.001 | 0.001 | — | — |
| | Day 16 | 0.001 | 0.001 | 0.001 | — |
| | Day 31 | 0.001 | 0.001 | 0.001 | 0.001 |
| | Pairwise comparisons | | | | |
| | using permutation | | | | |
| | MANOVAs on a | | | | |
| | distance matrix (fdr) | Day 0 | Day 3 | | |
| Figure | Day 3 | 0.001 | — | | |
| 15A | Day 5 | 0.001 | 0.001 | | |
| | Pairwise comparisons | | | | |
| | using permutation | | | | |
| | MANOVAS on a | | | | |
| | distance matrix (fdr) | CD | | | |
| Figure | HC | 0.001 | | | |
| 16A | | | | | |
| | Pairwise comparisons | | | | |
| | using permutation | | | | |
| | MANOVAs on a | | | | |
| | distance matrix (fdr) | CD | | | |
| Figure | HC | 0.001 | | | |
| 7B | | | | | |

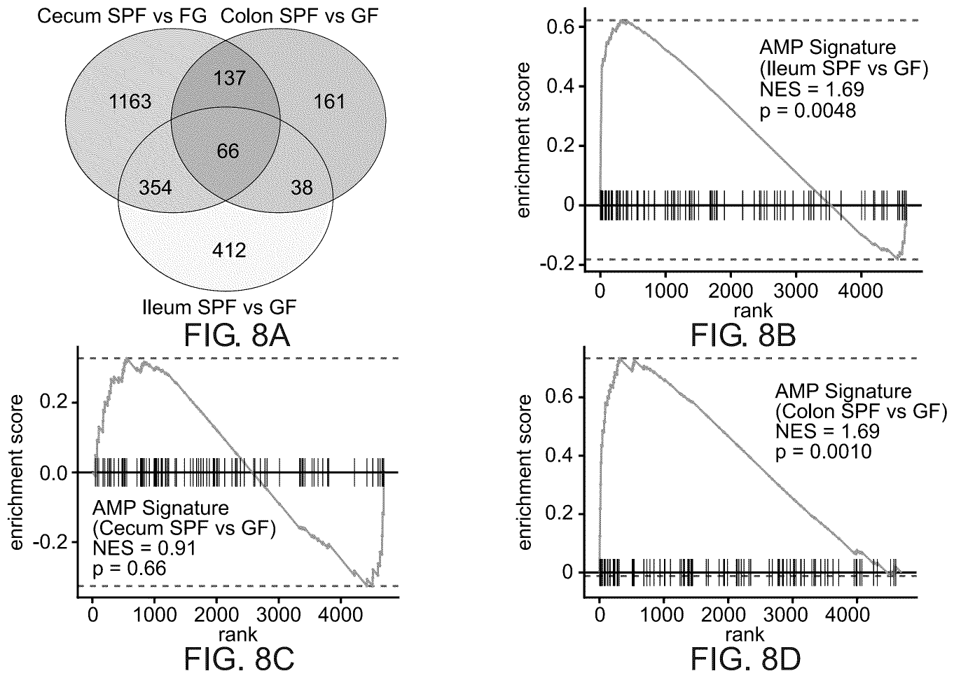

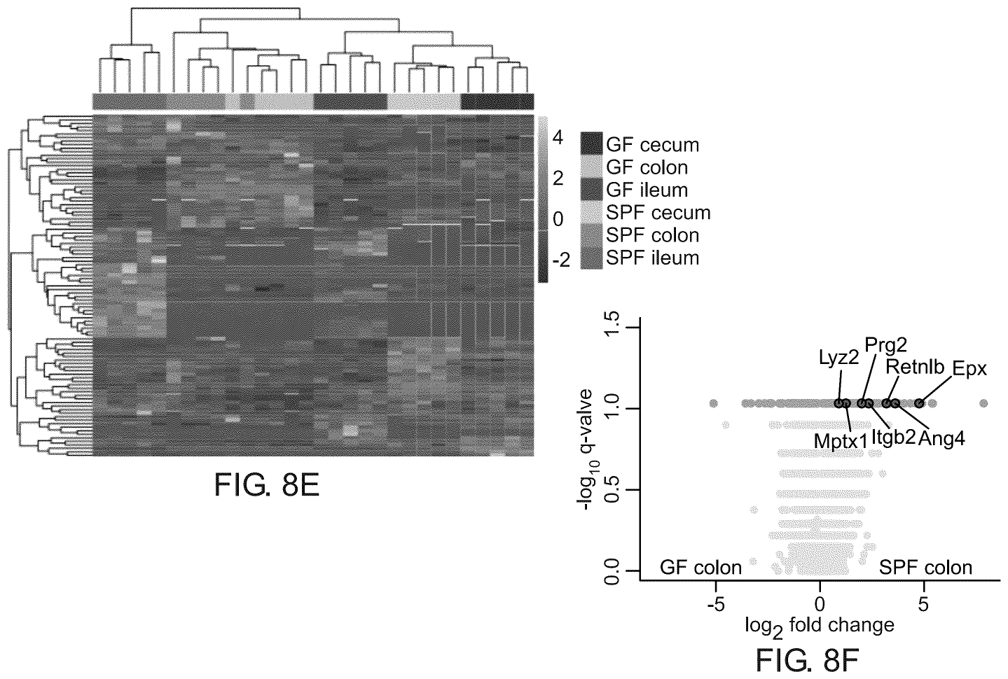

First, the proteomic landscape among different GI regions of SPF naïve mice was compared. 2087 differentially abundant proteins (q<0.10) between ileum and colon, 2156 between ileum and cecum, 1201 between colon and cecum were detected (FIG. 7C-E), suggesting a niche-specific proteomic configuration in the microbiome-colonized naïve setting. Interestingly, AMPs represented a prominent signature among the differentially abundant proteins between ileum and colon or cecum, but not between cecum and colon (FIG. 7F-H, Table S4). Specifically, relative to the cecum or colon, the ileum featured more differentially abundant AMPs derived from alpha-defensins (e.g, Defa20, 21, 22, 23, 24, Defa-rs1), the Reg3 family (Reg3b, Reg3g), granzymes (Gzma, Gzmb), Lysozyme C-1 (Lyz1), and less abundant Reg4 (FIG. 7C-D), while the AMP signature in SPF mice was less distinct between cecum and colon (FIG. 7E). The proteomic signature of each GI region between GF and SPF mice was then compared. Compared to GF mice, 1720, 870, and 402 differentially abundant proteins were identified in the cecum, ileum, and colon of SPF mice, respectively, and only 66 shared, differentially abundant proteins were noted along these three locations (FIG. 8A). Of note, AMPs represented an important changing signature among all the differentially abundant proteins between SPF and GF mice in the ileum and colon (FIG. 8B-D, Table S4). These results suggest that microbiome colonization along the GI tract modulates the proteomic landscape in a niche-specific manner, consistent with a previous study.

TABLE S4
AMP Profiles

| AMP Signature | | | | nMore | | |
|---|---|---|---|---|---|---|
| in comparisons | P val | ES | NES | Extreme | Size | LeadingEdge |
| SPF Ileum vs | 0.0046 | 0.6186 | 1.5728 | 3 | 97 | “Defa20” “Lgals2” “Itln1” |
| Colon | | | | | | “Defa21; Defa22” “Reg3b” “Nos2” |
| | | | | | | “Reg3g” “Defa-rs1” “Gzmb” |
| | | | | | | “Gzma” “Defa23” “Lyz1” |
| | | | | | | “Mmp7” “Apob” “Defa5” “Ido1” |
| | | | | | | “Defa9; Defa11; Defa6” “Asah2” |
| | | | | | | “Defa24” “Apoe” “Duoxa2” |
| | | | | | | “Elmo1” “Lgals9” “B2m” |
| | | | | | | “Apoa1” |
| SPF Ileum vs | 0.0013 | 0.6654 | 1.7922 | 0 | 97 | “Defa20” “Itln1” “Lgals2” |
| Colon | | | | | | “Defa21; Defa22” “Apob” “Nos2” |
| | | | | | | “Reg3b” “Mptx1” “Reg3g” “Defa- |
| | | | | | | rs1” “Gzma” “Lyz1” “Mmp7” |
| | | | | | | “Asah2” “Gzmb” “Defa23” |
| | | | | | | “Defa24” “Defa5” “Duoxa2” |
| | | | | | | “Ang4” “Ido1” |
| | | | | | | “Defa9; Defa11; Defa6” “Ngp” |
| SPF Cecum vs | 0.0703 | −0.4617 | −1.2648 | 21 | 97 | “Mptx1” “Ctse” “Apob” |
| Colon | | | | | | “Spink3” “Hgfac” “Chga” “Itln1” |
| | | | | | | “Ltf” “Gsn” “S100a11” “Ang4” |
| Ileum SPF vs | 0.0029 | 0.6216 | 1.7113 | 0 | 97 | “Nos2” “Reg3b” “Reg3g” “Gzma” |
| GF | | | | | | “Gzmb” “Ngp” “S100a9” “Mptx1” |
| | | | | | | “Chil3” “Duoxa2” “Defa5” |
| | | | | | | “Defa24” “Ltf” “Defa23” “Dmbt1” |
| | | | | | | “Mmp7” “Defa9; Defa11; Defa6” |
| | | | | | | “Defa-rs1” “Defa20” “Lyz1” “Itgb2” |
| | | | | | | “Lyz2” “Lcp1” “Defa21; Defa22” |
| | | | | | | “Hmgb1” “Fam49b” “Hmgb2” |
| | | | | | | “Pglyrp1” “Itgam” |
| Cecum SPF vs | 0.6524 | 0.3272 | 0.9105 | 3961 | 97 | “S100a9” “Chgb” “Gsdmc2” |
| GF | | | | | | “Hrg” “Vtn” “Retnlb” |
| | | | | | | “Pglyrp1” “Chga” “Isg15” |
| | | | | | | “Azgp1” “Gsdmdc1” “Orm1” |
| | | | | | | “Elmo1” “Ang4” “Chil3” “Ctsl” |
| | | | | | | “B2m” “Lyz2” “Hmgb1” |
| Colon SPF vs | 0.0012 | 0.7336 | 1.6888 | 0 | 97 | “Epx” “Ang4” “Retnlb” |
| GF | | | | | | “S100a9” “Itgb2” “Prg2” “Ear2” |
| | | | | | | “Itgam” “Mptx1” Apoh” |
| | | | | | | “Chil3” “Lyz2” “Vtn” “Azgp1” |
| | | | | | | “Ctsl” “Ear1” “Chga” “Ngp” |
| | | | | | | “Chgb” “Pglyrp1” “Clu” |
| | | | | | | “Lgals9” “Sepp1” “Orm1” |
| GF + mSFB vs | 0.0013 | 0.7352 | 1.9244 | 0 | 104 | “Nos2” “Reg3g” “Dmbt1” |
| GF + PBS | | | | | | “Gzmb” “Mptx1” “Duoxa2” |
| | | | | | | “S100a9” “Reg3b” “Gzma” |
| | | | | | | “Chil3” “Mmp7” “Defa-rs1” |
| | | | | | | “Isg15” “Lcn2” “Defa5” “Pltp” |
| | | | | | | “Mpo” “Apob” “Elmo1” |
| | | | | | | “Pon1” |
| GF + mSFB vs | 0.0012 | 0.6774 | 1.7286 | 0 | 104 | “Nos2” “Duoxa2” “Dmbt1” |
| GF + rSFB | | | | | | “Gzmb” “Mptx1” “Lcn2” |
| | | | | | | “S100a9” “Elmo1” “Gzma” |
| | | | | | | “Reg3g” “Chil3” “Defa5” |
| | | | | | | “Mmp7” “Isg15” “Iap” |
| | | | | | | “Ido1” “Defa-rs1” “Ngp” |
| | | | | | | “Mpo” “Fam49b” |
| GF + IL8 vs | 0.0012 | 0.7851 | 1.8212 | 0 | 91 | “Arg1” “Ang4” “Orm2” |
| GF + PBS | | | | | | “S100a9” “Ear2” “Itgb2” “Isg15” |
| | | | | | | “B2m” “Apcs” “Itgam” “Chil3” |
| | | | | | | “Cd5l” “Lcp1” “Spink3” “Saa1” |
| | | | | | | “Lyz2” “Apoa1” “Orm1” “Ear1” |
| | | | | | | “Pltp” “Epx” “Prg2” “Camp” |
| | | | | | | “Mptx1” “Fam49b” “Pon3” “Manf” |
| | | | | | | “Pon2” “Elane” “Ctsl” |
| Citrobacter | 0.0022 | 0.6151 | 1.5479 | 1 | 97 | “Ltf” “Ngp” “S100a9” “Lcn2” |
| day7 vs day0 | | | | | | “Chil3” “S100a8” “Mpo” “Camp” |
| | | | | | | “Cd177” “Itgb2” “Itgam” “Retnlg” |
| | | | | | | “Elane” “Dmbt1” “Elmo1” “Ctsg” |
| | | | | | | “Mmp9” “Epx” “Ido1” “Prg2” |
| | | | | | | “Pon1” “Apoa1” “Slpi” |
| Citrobacter | 0.0022 | 0.6262 | 1.6472 | 1 | 97 | “Ltf” “Ngp” “S100a9” “Lcn2” |
| day14 vs day0 | | | | | | “Cd177” “Chil3” “S100a8” |
| | | | | | | “Mpo” “Camp” “Itgb2” |
| | | | | | | “Itgam” “Retnlg” “Elmo1” “Ido1” |
| | | | | | | “Elane” “Epx” “Prg2” “Mmp9” |
| | | | | | | “Ear2” “Lcp1” “Fam49b” “B2m” |
| | | | | | | “Nos2” “Fn1” “Ctsg” “Pon2” |
| | | | | | | “Apoe” “Psap” “Isg15” |
| | | | | | | “Pglyrp1” “Gzma” “Pltp” |
| | | | | | | “Apoa1” “Arg1” “Gsdmdc1” |
| DSS day 5 vs | 0.0080 | 0.6930 | 1.2517 | 7 | 124 | “Apoa1” “Fn1” “Apoh” |
| day0 | | | | | | “Ngp” “Apoe” “Cd5l” |
| | | | | | | “Chil3” “Ltf” “Mpo” “Pon1” |
| | | | | | | “Clu” “Azgp1” “Apob” “Hrg” |
| | | | | | | “Thbs1” “Sepp1” “Lcn2” “Ace” |
| | | | | | | “Itgb2” “Adipoq” “Ctsb” “Gsn” |
| | | | | | | “Lcp1” “Manf” “Retnlg” |
| | | | | | | “Prtn3” “S100a9” “Apcs” |
| | | | | | | “Mbl1” “Mbl2” “Prg2” |
| | | | | | | “Camp” “Itgam” “Epx” “Vtn” |
| | | | | | | “Hgfac” “Pglyrp2” “Fcn1” |
| | | | | | | “Rnase4” “Ear2” “Serpina1” |
| | | | | | | “Habp2” “Spink3” “Mmp9” |
| | | | | | | “Isg15” “Lbp” “Elane” |
| | | | | | | “Apoa2” “Clec3b” |
| DSS day 16 vs | 0.0010 | 0.7577 | 1.8693 | 0 | 124 | “Apcs” “Mpo” “Ltf” |
| day0 | | | | | | “Orm2” “Ngp” “Itgb2” “Fn1” |
| | | | | | | “Chil3” “Orm1” “Vtn” |
| | | | | | | “Cd5l” “Apoh” “Apoa1” |
| | | | | | | “Ctsg” “Lcn2” “Lyz2” “Itgam” |
| | | | | | | “Prtn3” “Mmp9” “Lcp1” |
| | | | | | | “Retnlg” “Apob” “S100a9” |
| | | | | | | “Azgp1” “Elane” “Pon1” |
| | | | | | | “S100a8” “Apoe” “Camp” |
| | | | | | | “Chi311” “Adipoq” “Mbl1” “Clu” |
| | | | | | | “Hmgb2” “Thbs1” “Serpina1” |
| | | | | | | “S100a11” “Lbp” “Mbl2” |
| | | | | | | “Manf” “Prg2” “Ctsb” |
| | | | | | | “Cd177” “Sepp1” “Fam49b” |
| | | | | | | “Pltp” “Hrg” “Saa4” “Reg3a” |
| | | | | | | “Hgfac” “Reg2” “Saa2” |
| | | | | | | “Lypd8” “Habp2” |
| Pediatric | 0.0019 | 0.6717 | 1.6805 | 0 | 49 | “PRTN3” “AZU1” “S100A12” |
| Crohn's disease | | | | | | “CTSG” “S100A9” “LTF” |
| vs healthy | | | | | | “PRB3” “S100A8” “PGC” |
| controls | | | | | | “APCS” “PGLYRP1” “MPO” |
| | | | | | | “PRSS2” “RNASE2” “ELANE” |

To further explore the enteric AMP landscape, the known and potential AMPs derived from the discovery proteomic assessment were analyzed. Importantly, the AMP signature varied according to GI locations based on the first two principal components (PCs) (FIG. 1A, Table S3). Comparison of the AMP signature between SPF and GF mice revealed that the ileum featured the most distinct microbiome colonization-dependent AMP signature, as compared to the cecum and colon (FIG. 1A). Consistently, hierarchical clustering of samples based on the 97 detected AMPs revealed a location-specific and microbiome-dependent grouping (FIG. 8E). Indeed, a comparison between SPF and GF mice of all three GI regions revealed 36 differentially abundant AMPs (q<0.10) in the ileum, 29 in the cecum, and only 7 in the colon (FIG. 1B). Most of these AMPs were spatially distinct, as 25 out of the 36 AMPs were significantly altered only in the ileum, while 19 out of the 29 differentially abundant AMPs were cecum-specific (FIG. 1B). More specifically, relative to GF mice, unique AMPs with significantly increased abundance in the ileum of SPF mice included AMPs from the Reg3 family (Reg3b, Reg3g), alpha-defensins (e.g., Defa5, 20, 23, 24, Defa-rs1), granzymes, deleted in malignant brain tumors 1 protein (Dmbt1) and matrilysin (Mmp7, FIG. 1C). Unique AMPs significantly increased in the cecum of SPF mice compared to GF mice included chromogranin-A (Chga), chromogranin-B (Chgb), gasdermins (Gsdmdc 1, Gsdmc2), vitronectin (Vtn), histidine-rich glycoprotein (Hrg) and peptidoglycan recognition protein 1 (Pglyrp1) (FIG. 1D). In the colon, eosinophil peroxidase (Epx), angiogenin-4 (Ang4) and resistin-like beta (Retnlb) were the top differentially abundant AMPs in SPF mice in comparison to GF mice (FIG. 8F). Together, these results define a microbiome-dependent and region-specific AMP landscape along the healthy murine gut.

### Example II: AMP Signatures in the Colonized and the Diseased Gut

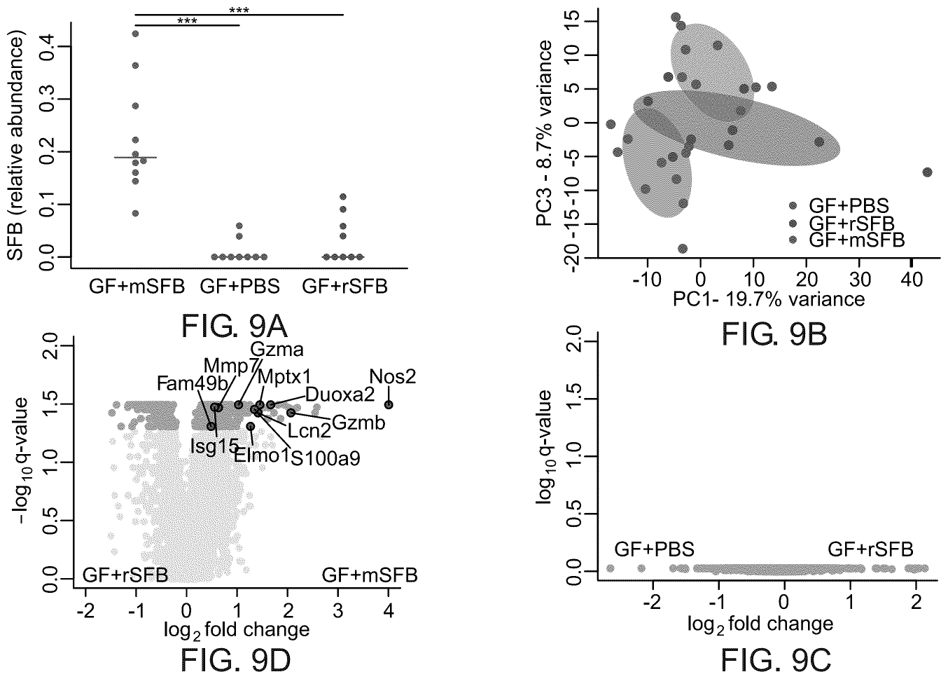

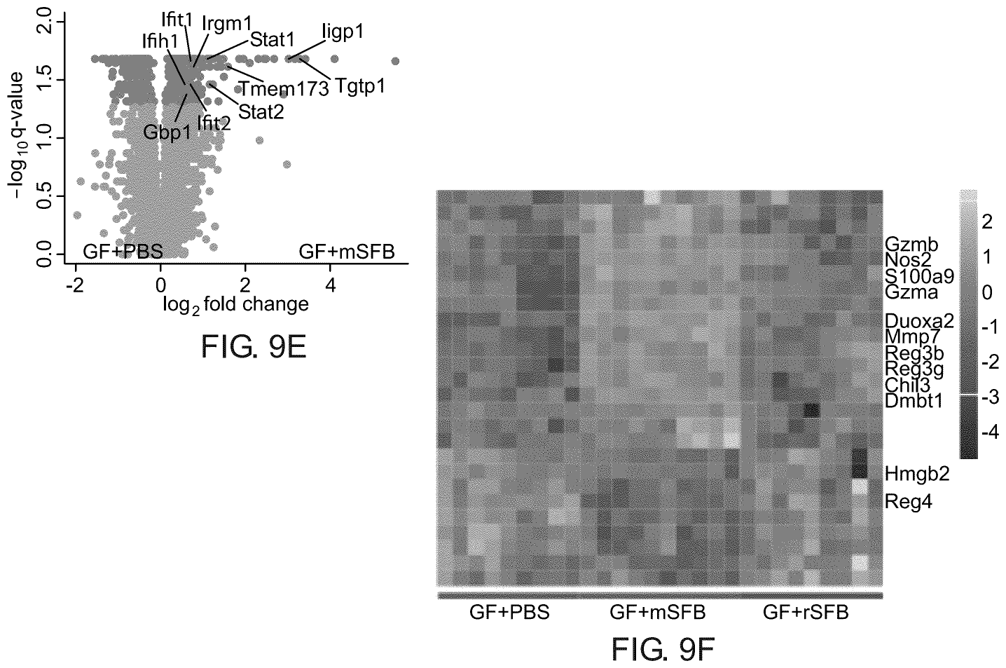

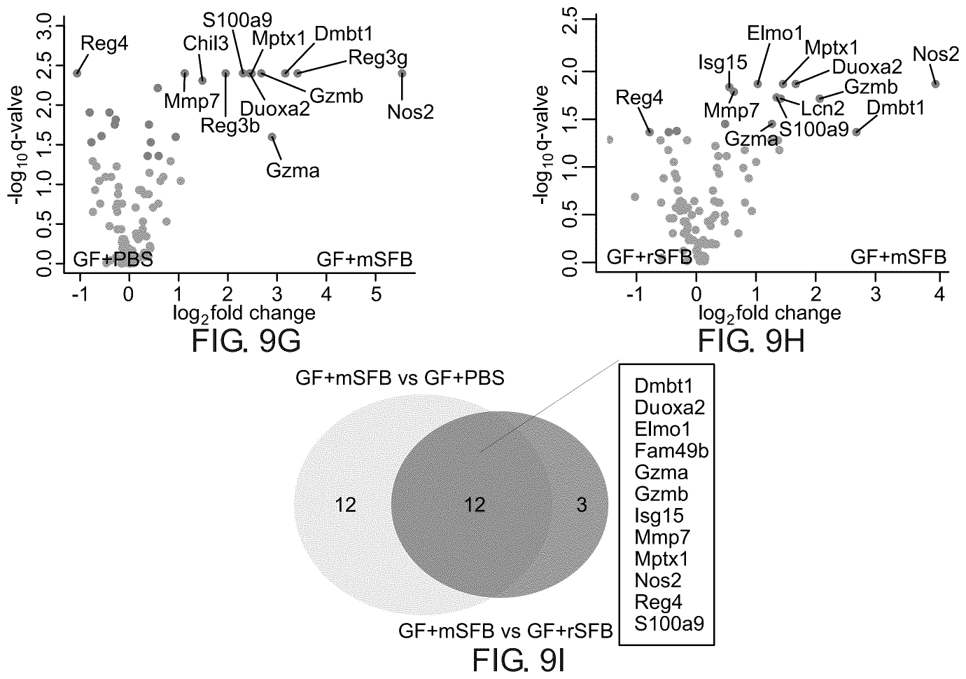

Proteomic AMP signatures in the commensal colonized gut. The induction of intestinal AMPs in the presence of gut microbiome prompted the exploration of relationships between the host AMP signature and specific commensal intestinal colonization patterns. First, the murine AMP responses upon mucosal attachment of a single commensal bacteria, segmented filamentous bacteria (SFB) were identified. Previously, SFB colonization in the murine ileum was shown to induce expression of genes associated with antimicrobial defense. 8-week-old C57BL6 GF mice were mono-colonized for 2 weeks with SFB indigenous to either mice (mSFB) or rats (rSFB), or with sterile vehicle (PBS) as a negative control (FIG. 1E). Of note, only mSFB has been reported to be capable of colonizing the terminal ileum (TI) in mice through adherence to ileal epithelial cells, which was also confirmed by metagenomic sequencing of TI mucosal samples (FIG. 9A). Overall, discovery analysis detected 5377 proteins from 63583 unique peptides by LFQ proteomics analysis of the TI mucosa. Notably, the global proteomic landscape in mSFB-inoculated GF mice clustered differently from the rSFB- or PBS-inoculated GF mice (FIG. 9B, Table S3). No differentially abundant protein could be detected between GF mice inoculated with rSFB and PBS (FIG. 9C), further highlighting the inability of rSFB to induce significant proteomic changes in the absence of significant epithelial cell engagement. Interestingly, AMPs were among the top differentially abundant proteins (q<0.05) upon mSFB colonization compared to PBS-(FIG. 1F) or rSFB-(FIG. 9D) inoculated GF mice (Table S4), indicating a potential role of AMPs in mSFB colonization. Besides, mSFB colonization induced a significant interferon signature in GF mice, as is evident from a significant increase of interferon-inducible proteins (e.g., Tgtp1, Iigp1, Irgm1, Gbp1, Tmem173, Stat1, Stat2, Ifih1, Ifih2, FIG. 9E).

AMP proteomic evaluation derived from the LFQ proteomic analysis of the TI of colonized mice demonstrated that mSFB mono-colonization led to a significant AMP induction as compared to GF mice inoculated with PBS or rSFB, as featured by distinct clustering of mSFB mice by PCA (FIG. 1G, Table S3), further highlighting the necessity of mucosal bacterial attachment in inducing AMP responses. Twenty-six out of the 104 detected known and potential AMPs were significantly altered among the three groups (q<0.05). Among them, the abundance of AMPs such as Reg3b, Reg3g, granzymes (Gzma, Gzmb), alpha-defensin-related sequence 1 (Defa-rs1), Dmbt1, Mmp7, and protein S100-A9 (S100a9) was significantly increased upon mSFB colonization. In contrast, the abundance of Reg4 and high mobility group protein B2 (Hmgb2) was significantly decreased in this colonization setting (FIG. 9F). To characterize the intersecting AMP signatures, the AMP signatures between mSFB- and PBS-inoculated mice were compared, revealing 24 differentially abundant AMPs (q<0.05, FIG. 9G), 12 of which were shared with the comparison between mSFB- and rSFB-inoculated mice (FIG. 9H-I). Utilizing a generalized additive model to unravel associations between the shifting AMP signature and commensal colonization patterns in the TI mucosa, it was found that the mucosa-attached SFB abundance measured by metagenomic sequencing explained 57.8% of the variance in the ileal AMP landscape (p<0.001, FIG. 1H). Furthermore, a Spearman correlation analysis revealed that 32 AMPs were significantly correlated with mucosal SFB abundance (q<0.05). The top 10 correlating AMPs were Dmbt1, Mmp7, Reg3g, chitinase-like protein 3 (Chil3), dual oxidase maturation factor 2 (Duoxa2), Reg3b, S100a9, engulfment and cell motility protein 1 (Elmo1), Reg4 and Hmgb2 (FIG. 1I). Consistently, the TI mucosal abundance of SFB was positively (Dmbt1, Reg3g, Mmp7, Chil3, S100a9, all p<0.001) or negatively (Reg4, p<0.001) correlated with specific individual AMP levels (FIG. 1J). Collectively, these results suggest that commensal mono-colonization and associated epithelial adhesion induces specific AMP responses in the TI mucosa, which in turn are strongly associated with the abundance of the mucosa-adherent commensals.

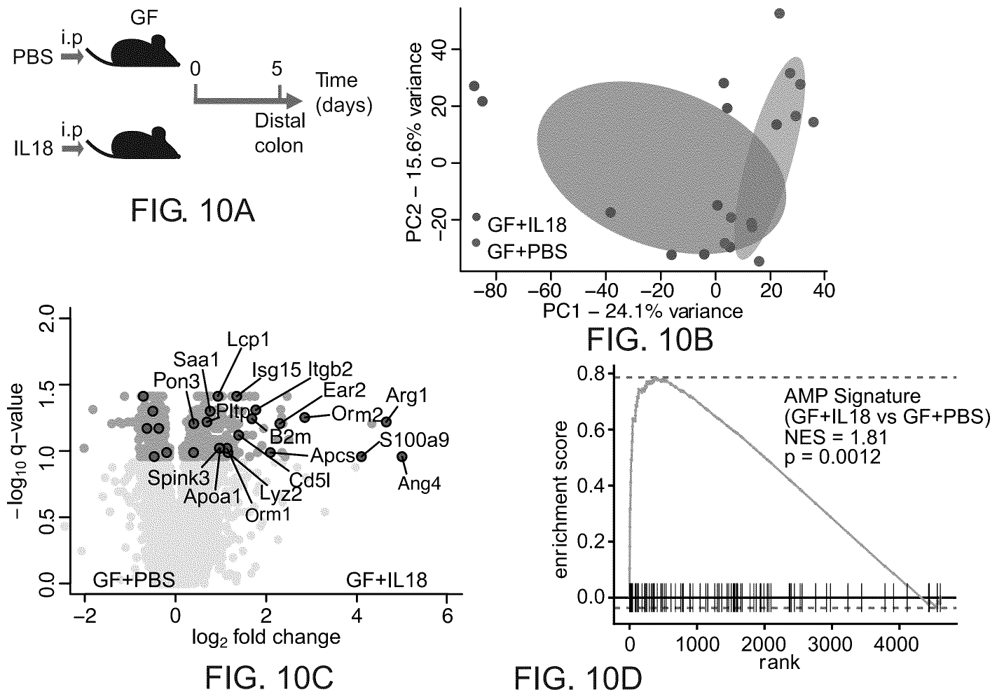

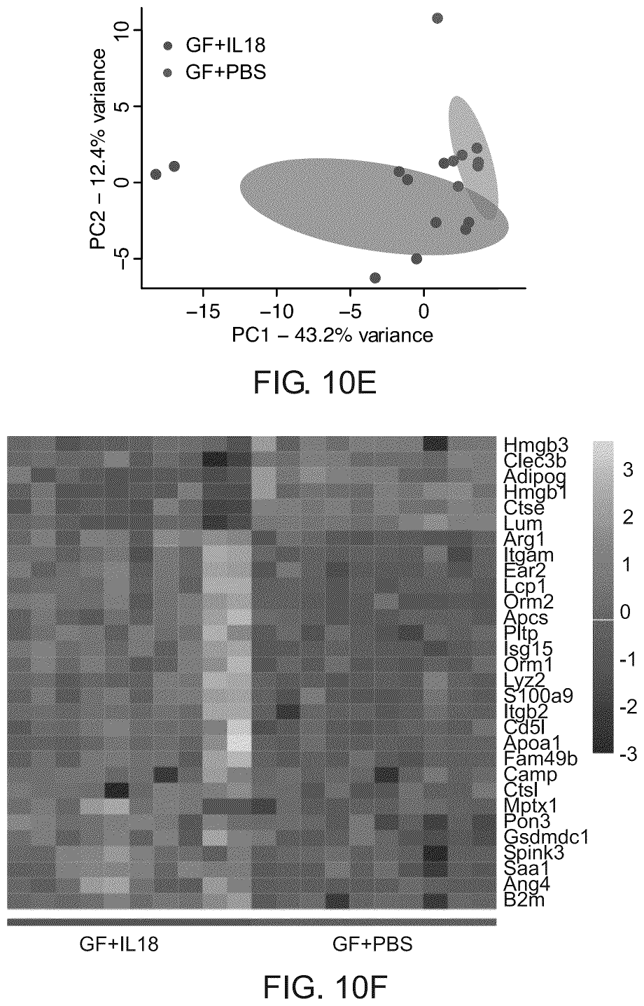

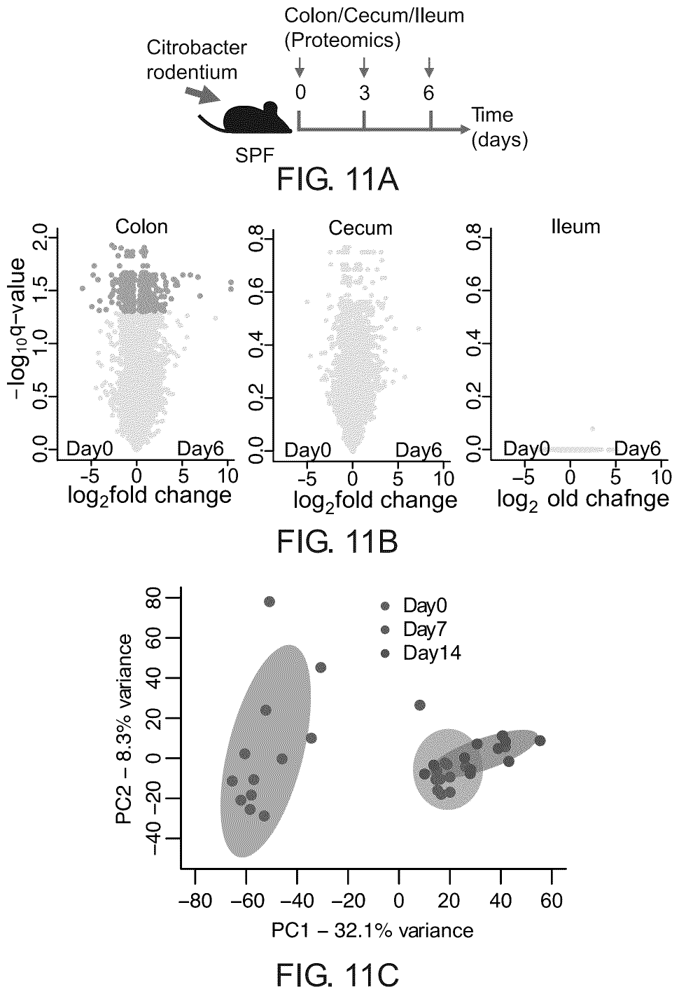

Commensal induction of the mucosal AMP landscape could also be indirectly mediated by the induction of intestinal innate immune signaling pathways. For example, it has been shown that commensal-mediated colonic interleukin-18 (IL18) production was both necessary and sufficient to regulate the transcript levels of some AMPs. Therefore, the proteomic pipeline was utilized to indicate whether IL18 induces AMP proteomic changes in homeostatic conditions. To this aim, recombinant IL18 or vehicle control (PBS) was administered to GF mice by intraperitoneal injection under sterile conditions (FIG. 10A). Of note, the landscape of colonic proteome upon IL18 administration, as measured by LFQ proteomic analysis, significantly differed from that of PBS-injected GF mice (FIG. 10B, Table S3). Among the differentially abundant proteins, AMPs represented an important changing signature (FIG. 10C-D, Table S4). Indeed, the AMP landscape derived from LFQ proteomics featured a clear separation between PBS- and IL18-injected GF mice, as shown by PCA (FIG. 10E, Table S3). In total, 30 out of the 91 known and potential AMPs detected in this experiment showed significantly altered abundance (q<0.10) upon IL18 supplementation, including angiogenin-4 (Ang4), S100a9, serum amyloid A-1 protein (Saa1), serum amyloid P-component (Apcs), alpha-1-acid glycoproteins (Orm1, Orm2) and lysozyme (Lyz2) (FIG. 10F). Together, these results suggest that commensal colonization of the gut mucosa may induce a robust alteration in the global AMP protein landscape, mediated by both microbial adhesion and an associated innate immune activation, as exemplified by IL18.

Pathogenic gut infection induces acute proteomic AMP signature shifts. Distinct changes of individual murine AMPs following enteric pathogenic invasion, such as Salmonella and Clostridium infection have been reported. To uncover the AMP landscape changes occurring during a pathogenic infection, 8-week-old C57BL6 SPF mice were orally infected with Citrobacter rodentium, an attaching and effacing pathogen which simulates human enteropathogenic and enterohaemorrhagic Escherichia coli infection. The discovery proteomics pipeline was applied to colonic, cecal and ileal samples to identify specific GI locations possibly affected by C. rodentium colonization (FIG. 11A). Supporting the generally accepted concept that C. rodentium mainly colonizes the colon, significant changes in the proteomic landscape in the colon were observed both at day 3 and day 6 post-infection, but not in the cecal or ileal regions during the same post-infection time points (FIG. 11B). Then, gut microbiome dynamics (based on sequencing of the 16S rRNA gene) and the colonic AMP responses at the peak of infection (day 7) and recovery (day 14) phases were analyzed in tandem (FIG. 2A). Indeed, C. rodentium outcompeted the other commensals in the colonic mucosa almost entirely at day 7 post-infection, and was eliminated during the restoration of the indigenous microbiome at day 14 (FIG. 2B-C). PCA of discovery LFQ proteomics (altogether 4695 proteins from 49399 unique peptides) illustrated differences of the proteomic landscape before (day 0) and during infection (day 7 and day 14) (FIG. 11C, Table S3). AMPs represented the top differentially abundant proteins (q<0.05) at day 7 compared to day 0, including increased levels of lactotransferrin (Ltf), neutrophilic granule protein (Ngp), S100a9, S100a8, neutrophil gelatinase-associated lipocalin (Lcn2), myeloperoxidase (Mpo), cathelicidin antimicrobial peptide (Camp), neutrophil elastase (Elane) and Dmbt1 (FIG. 2D, Table S4). Surprisingly, the altered AMP profile was persistent even following pathogen elimination at day 14 (FIG. 11D, Table S4), indicating a potentially important role of AMPs in both the peak and recovery phases of infection. Indeed, the colonic AMP configuration derived from LFQ proteomics featured clear separation among different infectious phases, as depicted by PCA (FIG. 2E, Table S3). Furthermore, the abundance of 80 out of the 97 detected known and potential AMPs were significantly altered among the three phases (q<0.05, FIG. 2F). A shared AMP signature, induced upon infection at day 7 and day 14 compared to day 0 (FIG. 11E), was identified, with most AMPs increasing at day 7 and persisting at day 14, while others significantly decreasing upon infection (FIG. 11F-G). Importantly, a generalized additive model indicated that the colonic mucosal load of C. rodentium was able to explain 73.3% of the variance in the colonic AMP signature (p<0.001, FIG. 2G). Spearman's correlation analysis identified 58 AMPs significantly associated with C. rodentium abundance in the colon (q<0.05), with the top 12 shown in FIG. 2H (absolute Rho>0.6).

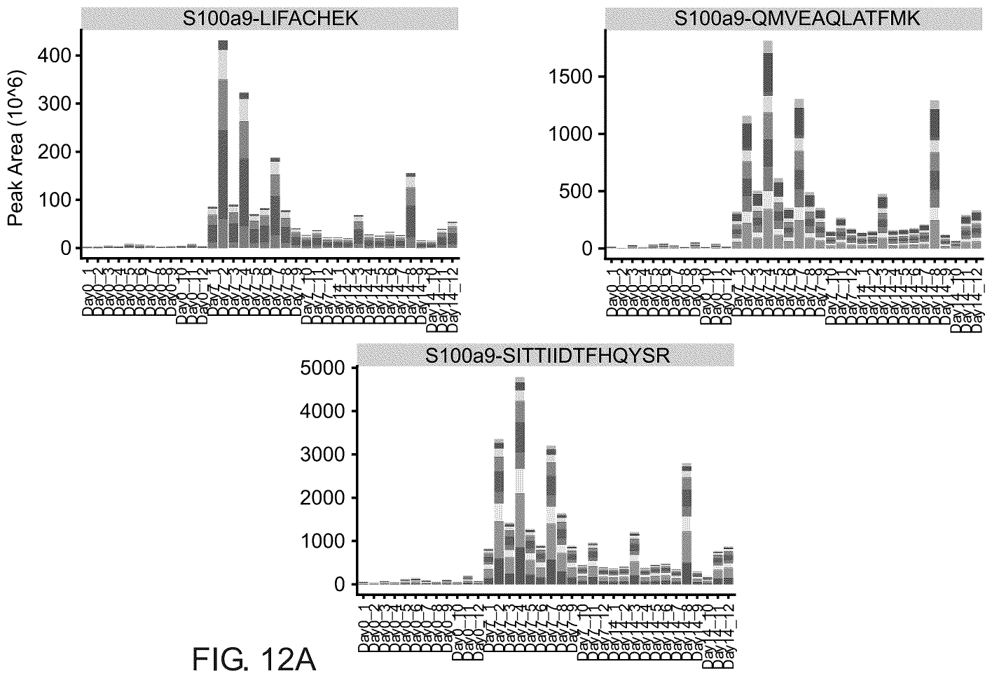

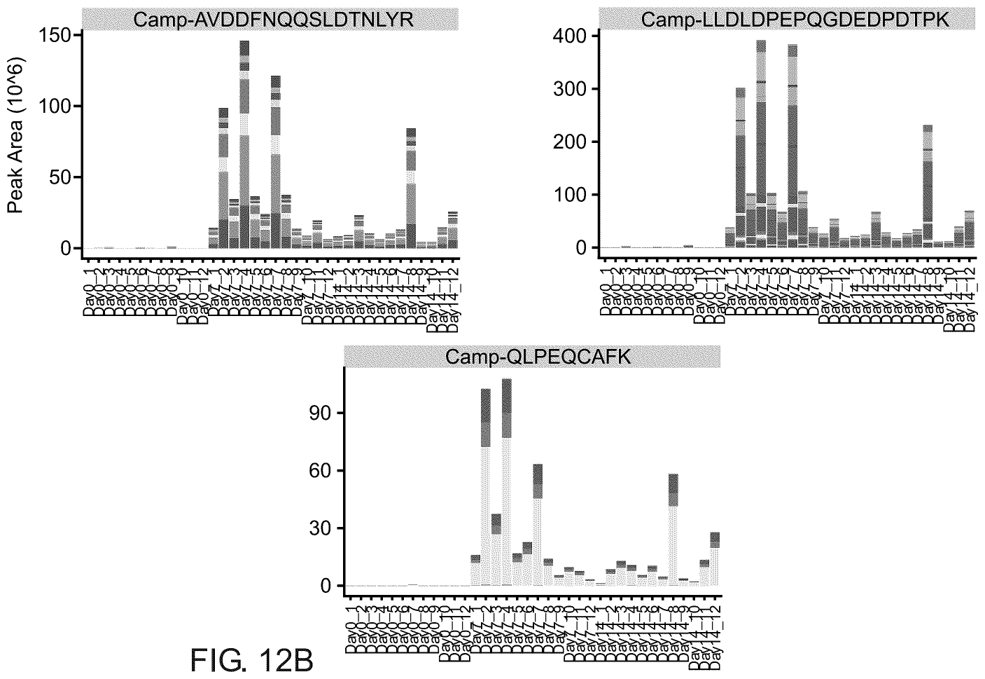

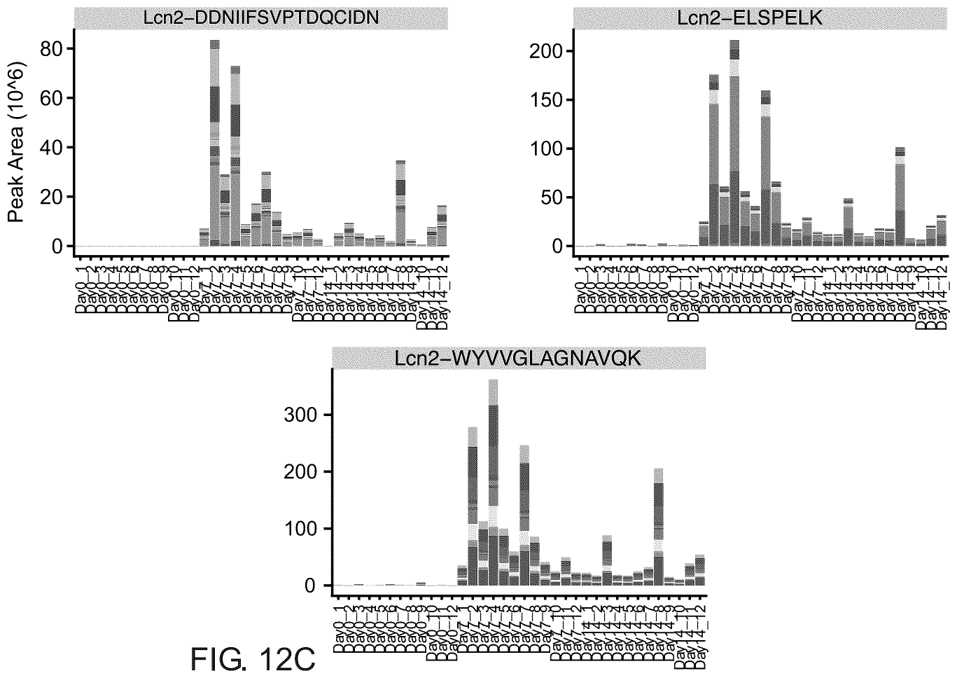

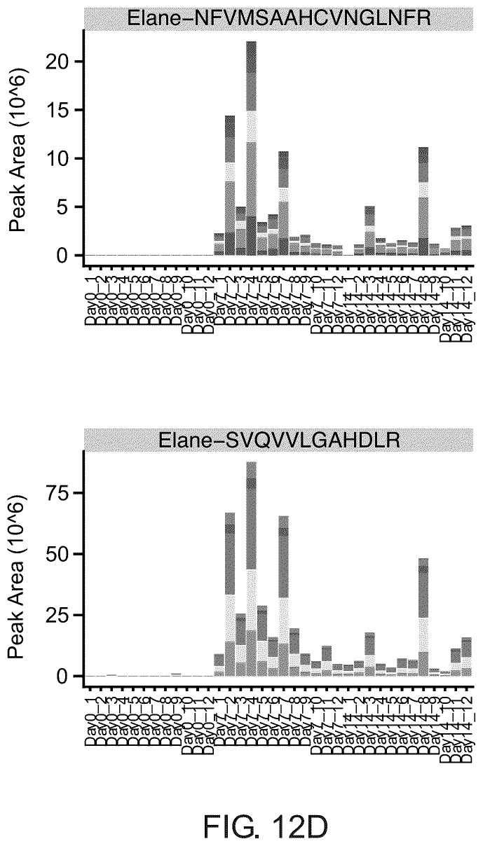

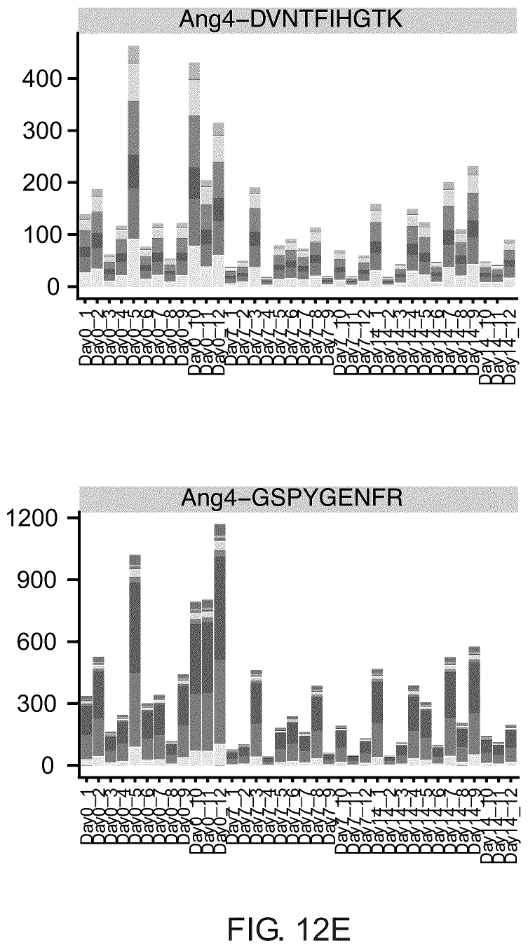

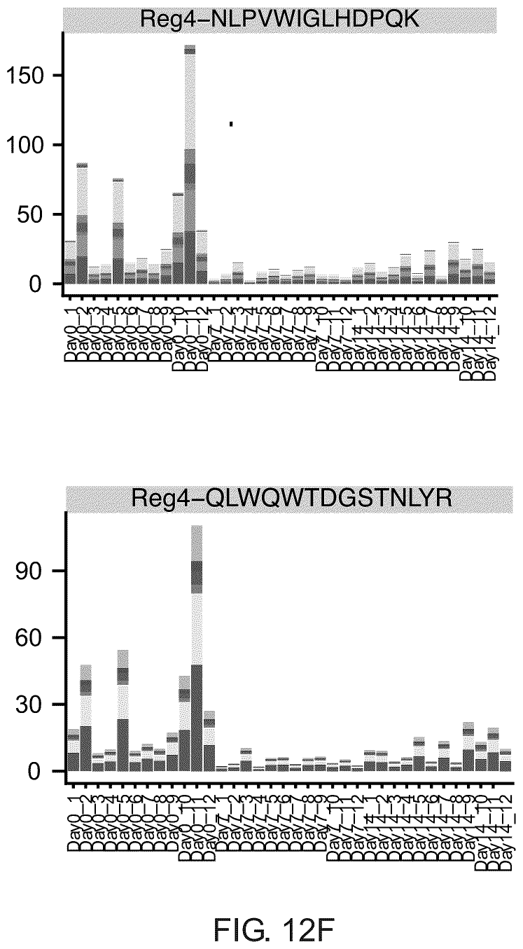

To further test the hypothesis that the AMP signature in the colon can be detected with an alternative non-invasive method, targeted proteomic analysis was undertaken on stool samples collected at the same infection phases as the mucosal samples (FIG. 2A). Tandem sequencing of these stool samples (by 16S rRNA gene analysis) demonstrated that fecal abundance of C. rodentium peaked at day 7, and became undetectable at day 14 (FIG. 11H). Of the 68 AMPs monitored in the PRM assay, tryptic peptides corresponding to 57 stool AMPs were detected and accurately quantified, including 15 AMPs in which synthetic SIL peptides were used as internal standards (FIG. 11I). PCA of the 57 targeted AMPs showed that the overall fecal AMP landscape at day 7 and day 14 was distinct from day 0 (FIG. 2I, Table S3), consistent with the colonic AMP signature noted above. In total, 30 AMPs featured significant alterations in abundance among different phases (q<0.05, FIG. 11J). These included S100a9, S100a11, Saa1, Reg3g which were increased, and Reg4, mucosal pentraxin (Mptx 1) and Ang4 which were decreased upon infection (FIG. 11I-J). Most of the significantly induced AMPs measured in stools were shared between day 7 and day 14 after infection (FIG. 11K-M). In the PRM assay, different tryptic peptides corresponding to the same AMP exhibited consistent changing patterns along the infectious course, further showcasing the robustness and reproducibility of targeted AMP quantification in stool. Representative examples included S100a9, Camp, Lcn2, Elane featuring an increased abundance, and Ang4, Reg4 featuring a decreased abundance during C. rodentium infection (FIG. 12A-F). Importantly, the fecal C. rodentium load was able to explain 31.9% of the variance in the global stool AMP landscape (p<0.001, FIG. 2J) and was strongly associated with multiple distinct AMPs (FIG. 2K). Notably, among the 16 fecal AMPs with the highest absolute correlation coefficients, 11 were also identified in the colon, including Mpo, Elane, S100a9, S100a8, Ltf, Chil3, Lcn2, Ngp, Camp, Dmbt1, Reg4 (FIG. 2K).

Based on these results, we next characterized the relationships between the colonic and fecal AMP features. Indeed, the colonic AMP signatures were significantly correlated with the global stool AMP landscape by Procrustes analysis (p<0.001, Correlation=0.64, FIG. 2L). Parallel comparisons of the differentially abundant AMPs between day 7 and day 0 revealed 17 shared AMPs in both the colon and stool (FIG. 2M), all of which displayed similar changing patterns upon infection at the peak and recovery phases (FIG. 2N). The abundance of individual colonic AMPs was also correlated with their fecal AMP abundance, exemplified by S100a9 and Reg4 (FIG. 11N). Together, these findings indicate that C. rodentium induces distinct AMP responses in both the colonic mucosa and the stool during the peak of infection, which are highly correlated with bacterial load, and persist even after pathogen clearance. Moreover, these results show that fecal proteomics can be utilized as a highly representative non-invasive proxy for proteomic detection of AMP dynamics in the colonic mucosa.

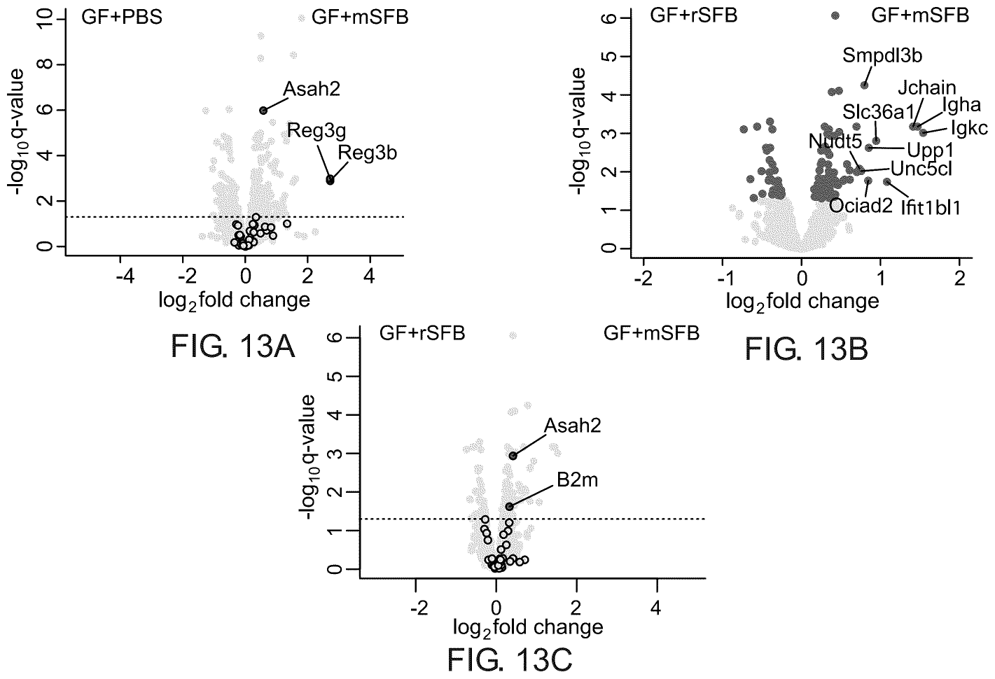

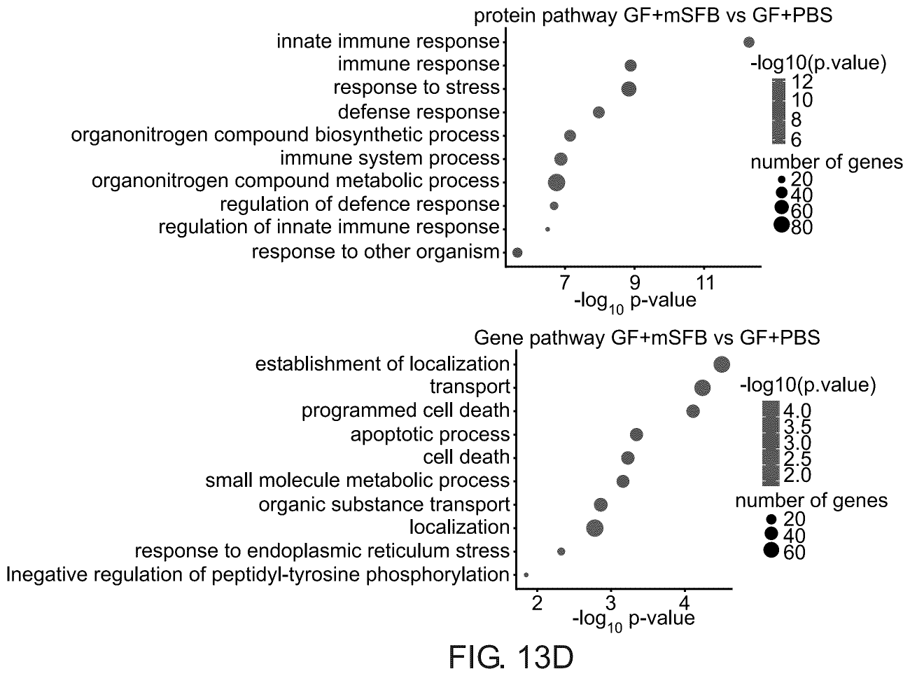

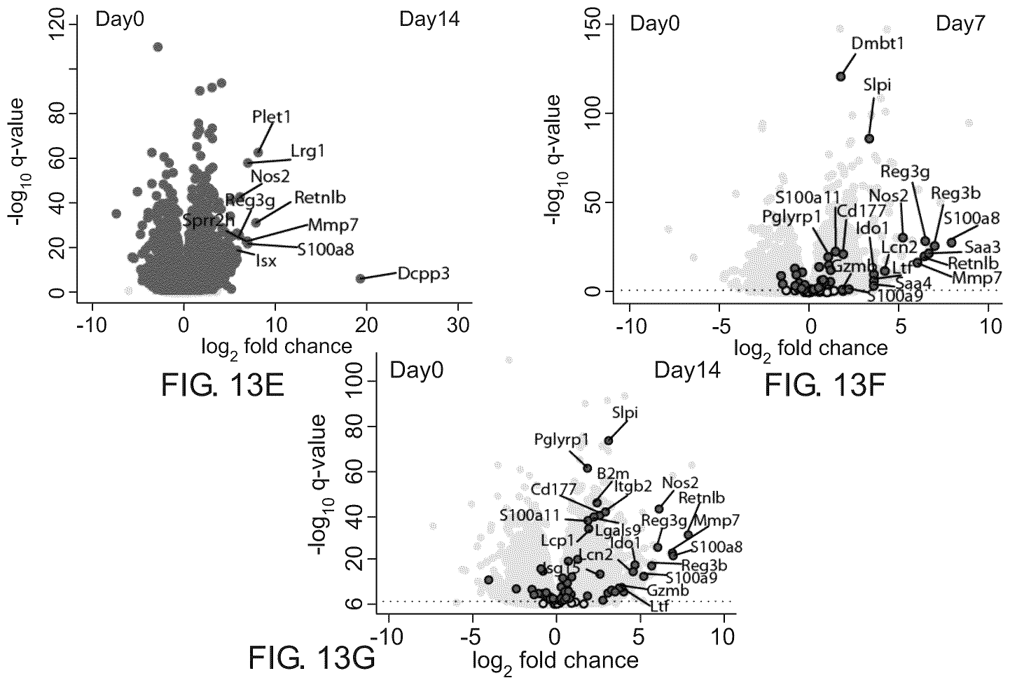

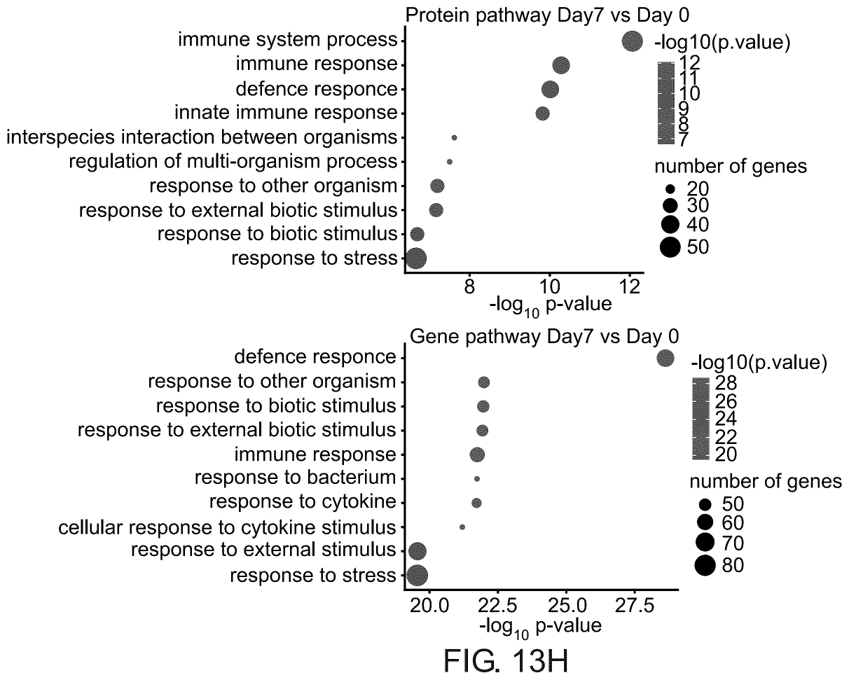

Proteomic AM P analysis is superior to RNA-based AMP characterization. The vast majority of previous AMP-related studies utilized mRNA assessment of individual AMPs extracted from the colonic mucosa(epithelial cell layer, to estimate their protein levels at different conditions. However, the expression and function of most AMPs are tightly regulated by diverse translational, post-translational and secretion-related mechanisms, partly to avoid non-specific damage to host cell membranes. To directly compare the utility of transcriptional AMP assessment to that obtained by the global proteomic pipeline, the host AMP transcriptional responses to intestinal commensal colonization or to pathogenic infection was defined and compared to the respective protein AMP responses. First, RNA-sequencing and analysis of transcripts from the TI mucosa collected 2 weeks after mSFB, rSFB, or vehicle control (PBS) inoculation in GF mice was performed. The transcriptomic landscape in mSFB-inoculated GF mice clustered slightly differently from the rSFB- or PBS-inoculated GF mice (FIG. 9A, Table S3). Both unique and shared TI transcriptional signatures were identified between GF mice colonized with mSFB and those colonized with rSFB or administered vehicle as controls (FIG. 3B). Overall, 338 differentially expressed transcripts (q<0.05) upon mSFB mono-colonization when compared to the PBS control (FIG. 3C) were identified. When focusing on the AMP-encoding transcripts, we found only 3 differentially expressed transcripts in the TI mucosa, namely Reg3b, Reg3g, and Asah2, upon mSFB colonization compared to PBS control (FIG. 13A), whose upregulation was also reported in the same context by a previous study. When comparing mSFB- and rSFB-inoculated GF mice, 131 differentially expressed transcripts were identified (FIG. 13B). Only two differentially expressed AMP-encoding transcripts (Asah2 and B2 m) were detected in mSFB-compared to rSFB-inoculated GF mice (FIG. 13C).

Further, the transcriptional signature with the discovery proteomic landscape measured upon mSFB colonization was compared. Notably, among the 24 differentially abundant AMPs detected by discovery proteomics (q<0.05), only two were also differentially expressed at the transcriptional level (Reg3b and Reg3g, FIG. 3D-E). Nineteen AMP-encoding transcripts displayed an increasing (e.g., Dmbt1, Duoxa2, Gzma, Gzmb, Mmp7, Mptxl, Nos2) or decreasing (e.g., Reg4, Hmgb2, Gm) trend upon mSFB attachment, similar to their corresponding proteomic changes, yet none reached statistical significance. Further, three AMP transcripts (S100a9, Chili, Defa-rs1) could not be detected by RNA-sequencing altogether (FIG. 3E). Consistent with the observation that the AMP response was better represented at the protein level, gene set enrichment analysis (GSEA) of genes derived from the differentially abundant proteins were mostly implicated in enriched pathways related to innate immune response and defense response (FIG. 13D). However, no significant enrichment of these pathways was observed, based on GSEA performed on RNA-sequencing data (FIG. 13D). These results indicate the superiority of proteomics over transcriptomics in characterizing the global AMP landscape in response to gut commensal colonization.

Next, a global transcriptional analysis of the colonic mucosal samples harvested before and during murine C. rodentium pathogenic infection was conducted. Mice infected with C. rodentium featured a colonic transcriptional landscape shift at day 7, which was distinct from that of uninfected mice, with this divergent signature sustained at day 14 (FIG. 3F, Table S3). Overall, 6371 differentially expressed colonic transcripts (q<0.05) were identified at day 7 compared to day 0, and 7888 identified at day 14 compared to day 0, among which 5188 transcripts were shared between the two post-infection time points (FIG. 3G-H, FIG. 13E). These results suggest that the induction of host transcriptional responses upon infection persisted after pathogen clearance, as was noted at the proteomic level (FIG. 11C). Interestingly, the AMP-encoding transcriptional responses represented important changing features among the overall differentially expressed transcripts at day 7 (FIG. 13F) and day 14 (FIG. 13G) post-infection, with the top AMP transcript hits shared by these two phases including S100a8, S100a9, Reg3b, Reg3g, Retnlb, Saa3, Lcn2, Slpi, and Ltf. Importantly, similar significantly enriched pathways were observed upon C. rodentium infection at the protein and transcriptional levels, both related to immune response and defense response (FIG. 13H).

Further, the similarities and differences between the transcriptional signature and the proteomic landscape at day 7 post-infection relative to day 0 were assessed. Both shared and unique differentially abundant AMPs (q<0.05) were identified at the transcriptional and protein level (FIG. 3I). Shared AMPs included 28 known and potential AMPs that were significantly altered by infection (e.g., S100a9, S100a8, Ltf, Dmbt1, Lcn2, Nos2, Slpi). Twenty-three AMPs featured significant alteration only at the transcriptional level, while they were not detected (e.g., Reg3b, Saa3, Saa4, Mmp7) or did not pass significance criteria (e.g., Gzmb, Pglyrp1, Reg3g, Retnlb, S100a11) in the LFQ proteomics analysis (FIG. 3J). Nonetheless, in the highly sensitive and accurate PRM assay on corresponding stool samples, Pglyrp1, S100a11, Saa1, and Reg3g were detected and found to be significantly more abundant in infected mice. The intensity of Reg3b, Retnlb, and Mmp7 remained unchanged based on stool PRM proteomic analysis, indicating unaltered secretion or activity of these AMPs at the post-transcriptional level. Notably, 36 AMPs were significantly changed in infected mice at the protein level, while remaining unchanged at the transcriptional level (e.g., Camp, Chil3, Mmp9, Mpo, Pon2, Prg2, Prtn3, Retnlg) or were undetectable by RNA-sequencing (e.g., Pon1, Ngp, Elane, Epx, Ctsg). Collectively, these results demonstrate that proteomic AMP detection and quantification is distinct and advantageous over transcriptional characterization of host AMP responses to intestinal commensal or pathogen colonization, due to higher sensitivity, superior representation of molecular function and activity, as well as wider applicability in different sample types (including both intestinal mucosa and stool).

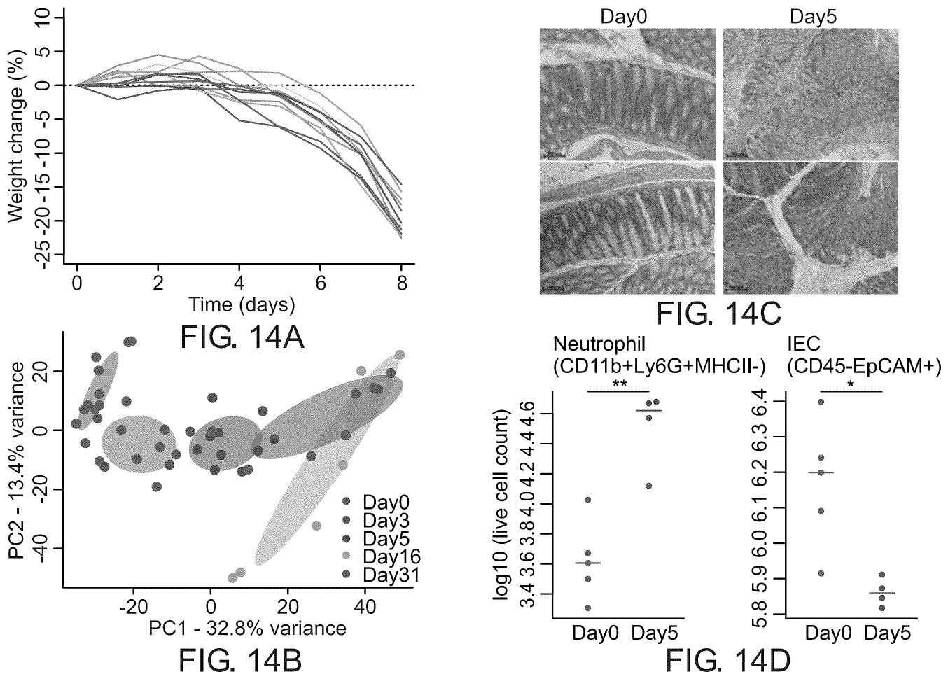

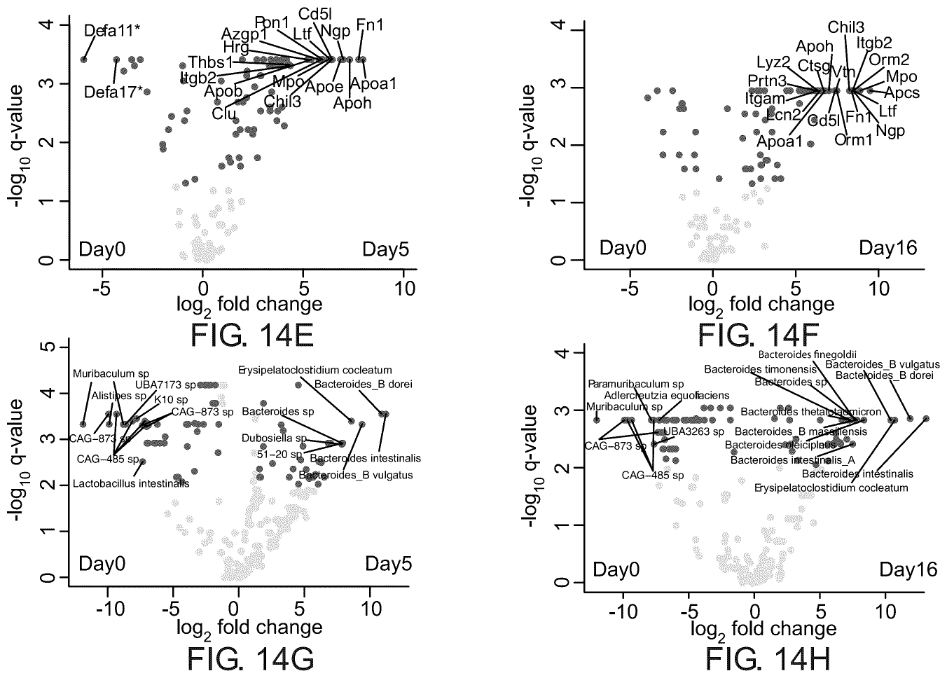

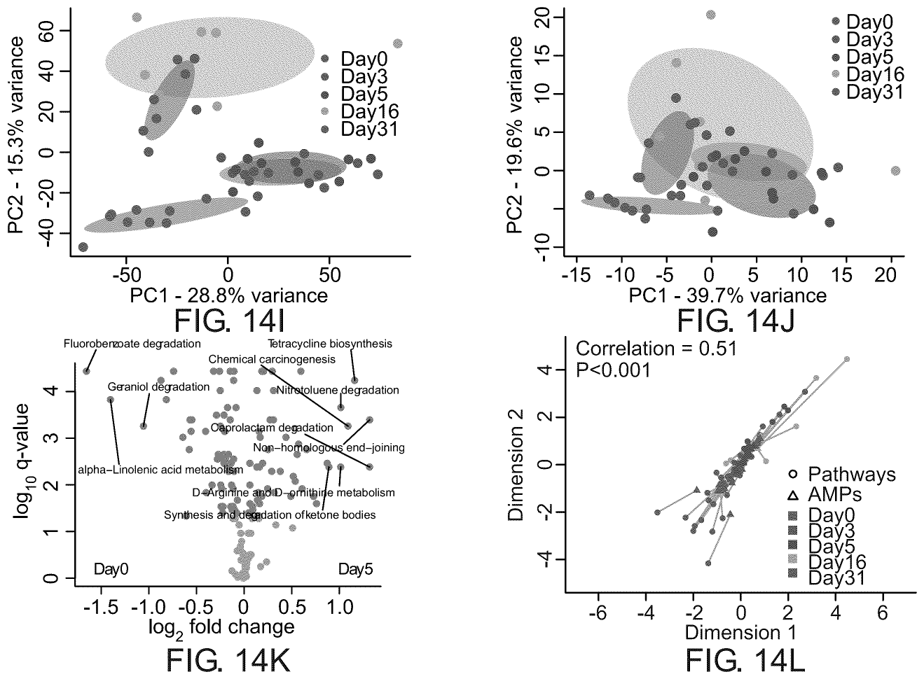

AMP dynamics are indicative of intestinal inflammation stages. To investigate the potential roles of the AMP landscape in contributing to disease-associated dysbiosis and consequent disease phenotypes, acute intestinal inflammation was induced through oral administration of dextran sulfate sodium (DSS) to mice. This murine DSS-induced colitis model is characterized by weight loss, bloody diarrhea, destruction of epithelial cells, and dysregulation of mucosal immune responses, thus resembling some key features of human IBD. To evaluate the AMP dynamics along disease course, stool discovery proteomics of samples collected before DSS exposure (day 0), at the early inflammatory phases (day 3 and 5), late inflammatory and tissue destruction phase (day 16), and at the end of the recovery phase (day 31, FIG. 4A) were performed. All mice featured individual-specific weight loss upon induction of DSS treatment (FIG. 4A, FIG. 14A). Overall, 1619 fecal murine proteins were identified from 13752 unique peptides by LFQ analysis. The proteomic landscape featured a time-dependent trend over the first PC, showing a continuous process from day 0 through early inflammatory phases (day 3 and day 5) to late inflammatory (day 16) and the end of recovery phase (day 31, FIG. 14B, Table S3). Of note, AMPs were among the most significantly enriched proteins (q<0.05) at the early inflammatory phase (day 5, FIG. 4B, Table S4) and even more so at the late inflammatory stage (day 16, FIG. 4C, Table S4), further highlighting the potentially important role of AMP alterations in the context of intestinal inflammation and mucosal healing.

Next the AMP signatures based on known and candidate AMPs detected by the LFQ analysis were examined. Similar to the proteomic landscape, the global AMP landscape featured time-dependent alterations across PC1, coupled with PC2 distinctions between the AMP landscape in early pre-clinical phases (day 3 and day 5), the pre-induction period (day 0) and later phases (day 16 and day 31, FIG. 4D, Table S3). Out of 124 detected AMPs, 99 were significantly altered among the different phases of DSS-induced colitis (q<0.05) with differential dynamics patterns noted (FIG. 4E), suggesting an extensive and complex alteration of the AMP signature throughout colitis and recuperation. Interestingly, LFQ analysis also demonstrated a significant increase in the fecal abundance of the neutrophil marker lymphocyte antigen 6 complex locus G6D (Ly6G) and a decrease in the goblet cell marker mucin-2 (Muc2) at day 5 following DSS consumption (FIG. 4F), which serve as markers of prominent neutrophil lamina propria infiltration and extensive intestinal epithelial cells (IEC) damage, respectively, during early colonic inflammation (day 5, FIG. 14C). This was further confirmed by elevated colonic CD11b+Ly6G+MHCII-neutrophil counts and reduced EpCAM+CD45-IEC counts, as analyzed by flow cytometry (FIG. 14D). In agreement with these cellular patterns, the fecal abundance of polymorphonuclear leukocyte (PMN)-produced AMPs was increased upon induction of acute DSS colitis, including the neutrophil-produced proteins Camp, Lcn2, Ltf, Mpo, Ngp, proteinase 3 (Prtn3), Elane, cathepsin G (Ctsg), Pglyrp1, S100a8 and S100a9, as well as eosinophil-generated AMPs, including eosinophil cationic protein 2 (Ear2), eosinophil peroxidase (Epx), bone marrow proteoglycan (Prg2) (FIG. 4G). In contrast, multiple IEC-produced AMPs were reduced following the induction of acute colitis, including Ang4, intelectin-1 (Itini), galectin 4 (Lgals4), Mptx 1, Retnlb, and zymogen granule membrane protein 16 (Zg16), all staying suppressed throughout disease and recovery phases. Other IEC-produced AMPs, including phospholipase A2 (Pla2g1b) and Reg proteins (Reg3a, Reg3g, Reg4), were significantly reduced at early DSS course, but recovered at later disease phases (FIG. 4H).

The alpha defensins of both IEC and PMN origin, mostly featured decreased abundance in the early disease phases, while recovering in the recovery phase (FIG. 4G-H).

In addition to PMN- and IEC-produced AMPs, other top differentially abundant AMPs upon early (day 5, FIG. 14E) and late (day 16, FIG. 14F) colitis included lipoproteins (Apoa1, Apoe, Apoh, Clu, Apob), extracellular matrix-associated peptides (fibronectin, vitronectin) and Hrg, which have been reported to possess antimicrobial activities. Importantly, DSS-induced weight loss in mice, a proxy for colitis severity, was strongly correlated with early pre-clinical fecal AMP landscape dynamics (day 5 compared to day 0), using a linear regression model (p=0.027, R\hat{ }2=0.37, FIG. 4I), indicating the potential of early-phase fecal AMP signatures to constitute non-invasive parameters associated with evolving clinical features in gut inflammatory disease.

As DSS-induced colitis is also hallmarked and driven by marked microbiome dysbiosis, we next sought to assess the relationships between fecal AMPs and microbiome dynamics upon DSS challenge. Based on metagenomics sequencing, the fecal microbiome composition in DSS treated-mice became distinct from untreated mice at both early DSS phases (day 3 and day 5) and later phases (day 16 and day 31, FIG. 4J, Table S3). The abundance of commensal bacterial genera, namely Muribaculum, Paramuribaculum, Lactobacillus, Prevotella, Akkermansia, CAG-485, and CAG-873 was significantly decreased after induction of DSS colitis, and was only partly restored upon recovery (FIG. 4K, Table S5). Conversely, the abundance of bacterial genera such as Bacteroides_B, Bacteroides and Parabacteroides featured a significant increase upon DSS exposure, which persisted until the weight recovery phase (FIG. 4K, Table S5).

TABLE S5

| Bacterial | day3 vs day 0 | | | day5 vs day0 | | | day16 vs day0 | | |
|---|---|---|---|---|---|---|---|---|---|
| genus | p_value | q_value | FC | p_value | q_value | FC | p_value | q_value | FC |
| g_{——}Muribaculum | 0.0078 | 0.0286 | −3.5393 | 0.0078 | 0.0319 | −2.8851 | 0.0078 | 0.04079 | −4.1438 |
| g_{——}Paramuribaculum | 0.0078 | 0.0286 | −1.4449 | 0.0078 | 0.0319 | −2.1007 | 0.0078 | 0.04079 | −2.5998 |
| g_{——}CAG-485 | 0.0078 | 0.0286 | −1.5208 | 0.0078 | 0.0319 | −0.9153 | 0.1953 | 0.41725 | −1.5571 |
| g_{——}Duncaniella | 0.1953 | 0.2817 | 0.09102 | 0.5468 | 0.6310 | −0.2348 | 0.0078 | 0.04079 | −2.2357 |
| g_{——}UBA3263 | 0.1953 | 0.2817 | 0.18528 | 1 | 1 | −0.2368 | 0.0078 | 0.04079 | −3.6597 |
| g_{——}UBA7173 | 0.0078 | 0.02864 | −3.21581 | 0.0078 | 0.0319 | −1.2214 | 0.0078 | 0.04079 | −1.6820 |
| g_{——}CAG-873 | 0.0078 | 0.02864 | −3.18078 | 0.0078 | 0.0319 | −2.3613 | 0.0078 | 0.04079 | −4.5751 |
| g_{——}Bacteroides | 0.0078 | 0.02864 | 2.83587 | 0.0156 | 0.0502 | 1.9019 | 0.0078 | 0.04079 | 2.23302 |
| g_{——}Prevotella | 0.0078 | 0.02864 | 4.46821 | 0.0078 | 0.0319 | −3.5585 | 0.3828 | 0.66637 | −0.4791 |
| g_{——}Bacteroides_B | 0.0078 | 0.02864 | 2.70213 | 0.0078 | 0.0319 | 3.2606 | 0.0078 | 0.04079 | 4.51933 |
| g_{——}Parabacteroides | 0.0781 | 0.15625 | −0.62077 | 0.7422 | 0.8246 | 0.2181 | 0.0156 | 0.07343 | 1.70414 |
| g_{——}RC9 | 0.0390 | 0.09290 | 1.68345 | 0.9453 | 1 | −0.0033 | 0.0234 | 0.08812 | 2.2219 |
| g_{——}COE1 | 0.0390 | 0.09290 | 0.94696 | 0.1093 | 0.1789 | 1.01767 | 1 | 1 | 0.0680 |
| g_{——}Akkermansia | 0.0078 | 0.02864 | −3.02105 | 0.6406 | 0.7298 | 0.36214 | 0.3125 | 0.61197 | 0.5942 |
| g_{——}Lactobacillus | 0.03461 | 0.09290 | −4.19423 | 0.0519 | 0.1144 | −1.5386 | 0.1953 | 0.41725 | 2.5555 |

| Bacterial | day31 vs day0 | | | day16 vs day5 | | | day31 vs day5 | | |
|---|---|---|---|---|---|---|---|---|---|
| genus | p_value | q_value | FC | p_value | q_value | FC | p_value | q_value | FC |
| g_{——}Muribaculum | 0.0078 | 0.0347 | −1.2549 | 0.25 | 0.4151 | −1.2512 | 0.1093 | 0.1835 | 1.6314 |
| g_{——}Paramuribaculum | 0.0078 | 0.0347 | −1.2578 | 0.5468 | 0.7059 | −0.5031 | 0.0156 | 0.0611 | 0.8348 |
| g_{——}CAG-485 | 0.9453 | 1 | 0.08707 | 0.5468 | 0.7059 | −0.6283 | 0.0078 | 0.0407 | 1.0044 |
| g_{——}Duncaniella | 0.0078 | 0.0347 | −1.13960 | 0.0078 | 0.0660 | −2.0030 | 0.0156 | 0.0611 | −0.9060 |
| g_{——}UBA3263 | 0.0234 | 0.0719 | 1.48228 | 0.0078 | 0.0660 | −3.4277 | 0.0156 | 0.0611 | −1.2509 |
| g_{——}UBA7173 | 0.1484 | 0.2935 | −0.5343 | 0.7421 | 0.8964 | −0.4796 | 0.0234 | 0.0688 | 0.6869 |
| g_{——}CAG-873 | 0.0078 | 0.0347 | −1.2827 | 0.4609 | 0.6212 | −2.2327 | 0.1093 | 0.1835 | 1.0771 |
| g_{——}Bacteroides | 0.0078 | 0.0347 | 1.6522 | 0.6406 | 0.7839 | 0.3339 | 0.5468 | 0.5977 | −0.2506 |
| g_{——}Prevotella | 0.3125 | 0.4795 | −0.3328 | 0.0156 | 0.1037 | 3.0853 | 0.0078 | 0.0407 | 3.2419 |
| g_{——}Bacteroides_B | 0.0078 | 0.0347 | 4.1730 | 0.25 | 0.4151 | 1.2549 | 0.0078 | 0.0407 | 0.9089 |
| g_{——}Parabacteroides | 0.0078 | 0.0347 | 2.1078 | 0.0234 | 0.1089 | 1.4845 | 0.0390 | 0.0895 | 1.8864 |
| g_{——}RC9 | 0.1093 | 0.22638 | 1.1201 | 0.3125 | 0.5010 | 2.1947 | 0.6406 | 0.6843 | 1.1216 |
| g_{——}COE1 | 0.0234 | 0.07192 | −0.8161 | 0.25 | 0.4151 | −0.9441 | 0.0078 | 0.0407 | −1.8262 |
| g_{——}Akkermansia | 0.0156 | 0.06046 | 1.9974 | 0.4609 | 0.6212 | 0.2269 | 0.0781 | 0.1562 | 1.6283 |
| g_{——}Lactobacillus | 0.0390 | 0.10864 | 2.5429 | 0.0078 | 0.0660 | 4.1153 | 0.0078 | 0.0407 | 4.1081 |

The most expanded species included Bacteroides intestinalis, Bacteroides_B dorei, Bacteroides_B vulgatus, and Erysipelatoclostridium cocleatum at both day 5 (FIG. 14G) and day 16 (FIG. 14H), whereas the most suppressed taxa were Muribaculum, CAG-873, and CAG-485 species. Fecal microbiome functions, represented by the KEGG orthologs (KO) genes (FIG. 14I, Table S3) and KEGG pathways (FIG. 14J, Table S3), displayed pattern alterations consistent with the dysbiotic microbiome composition, including pathways related to tetracycline biosynthesis, nitrotoluene degradation and caprolactam degradation at the early inflammatory phase at day 5 (FIG. 14K). Importantly, throughout disease course, the landscape of fecal AMPs was significantly correlated with fecal microbiome composition (FIG. 4L, correlation=0.69, p<0.001) and function (FIG. 14L, correlation=0.51, p<0.001) by Procrustes analysis. This demonstrates a high concordance between fecal AMP features and colitis-associated microbiome alterations in this highly dynamic inflammatory setting.

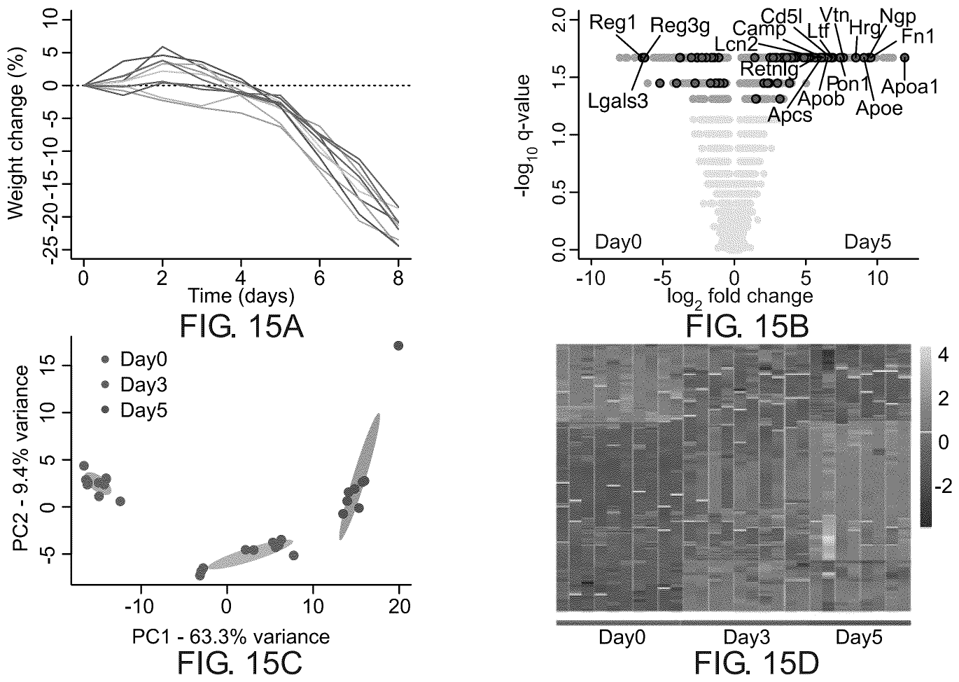

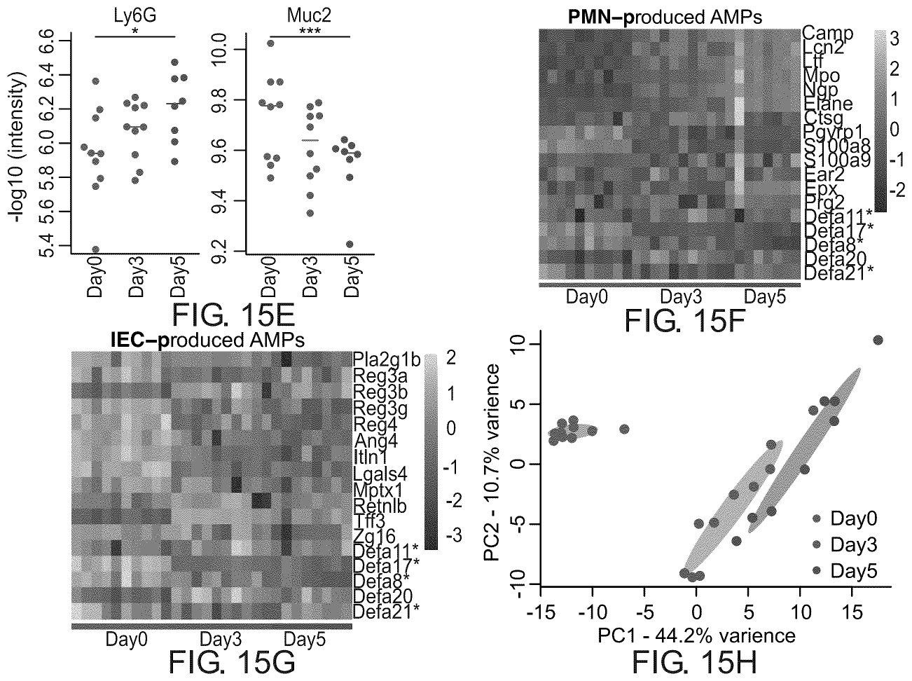

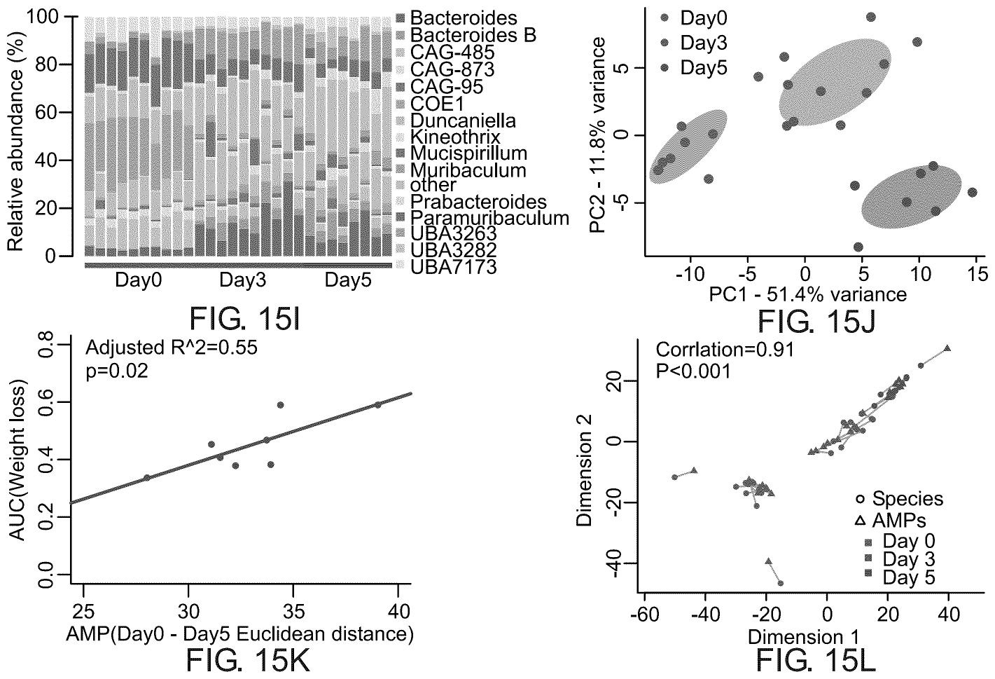

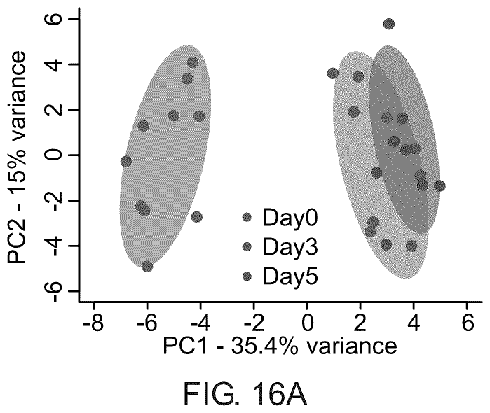

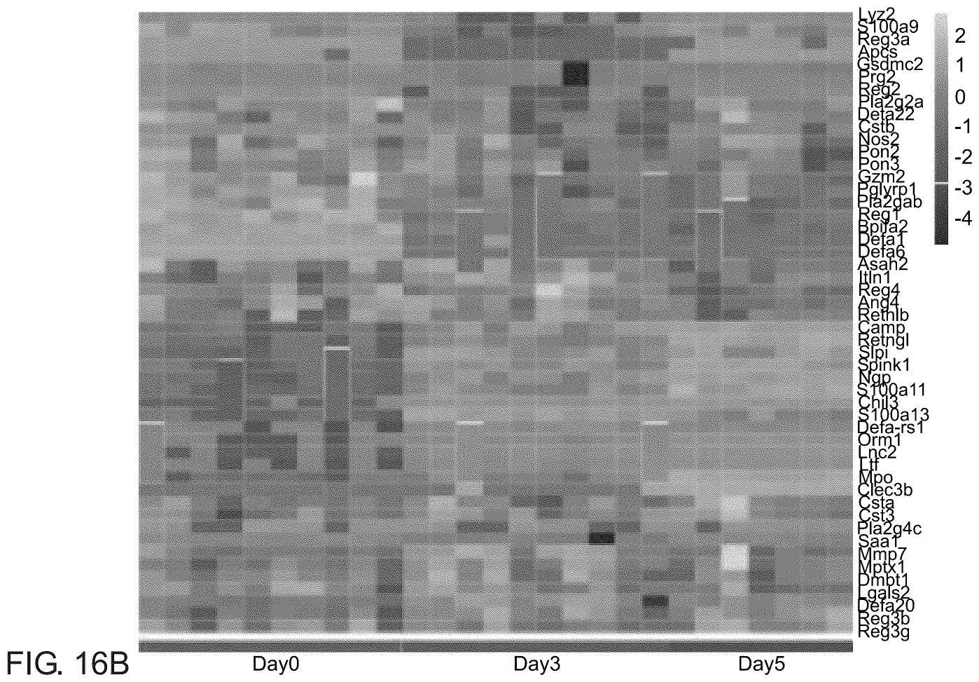

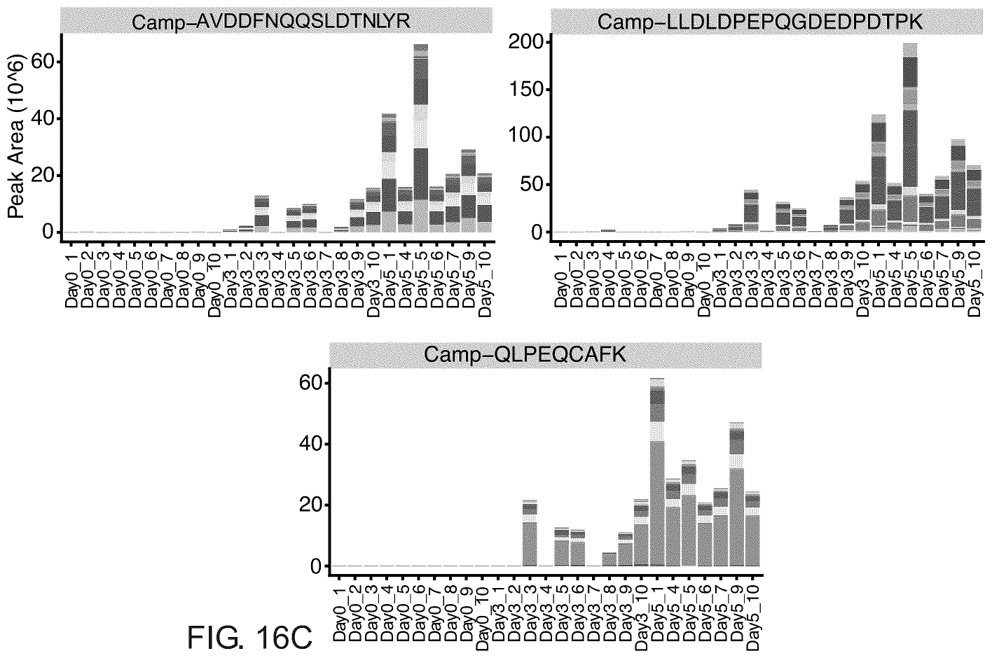

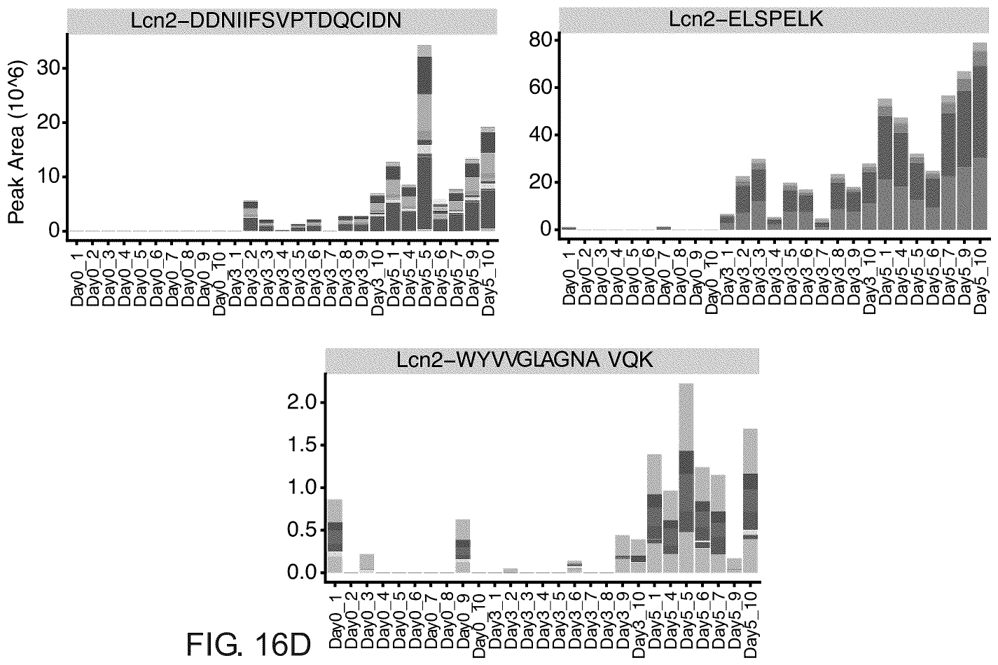

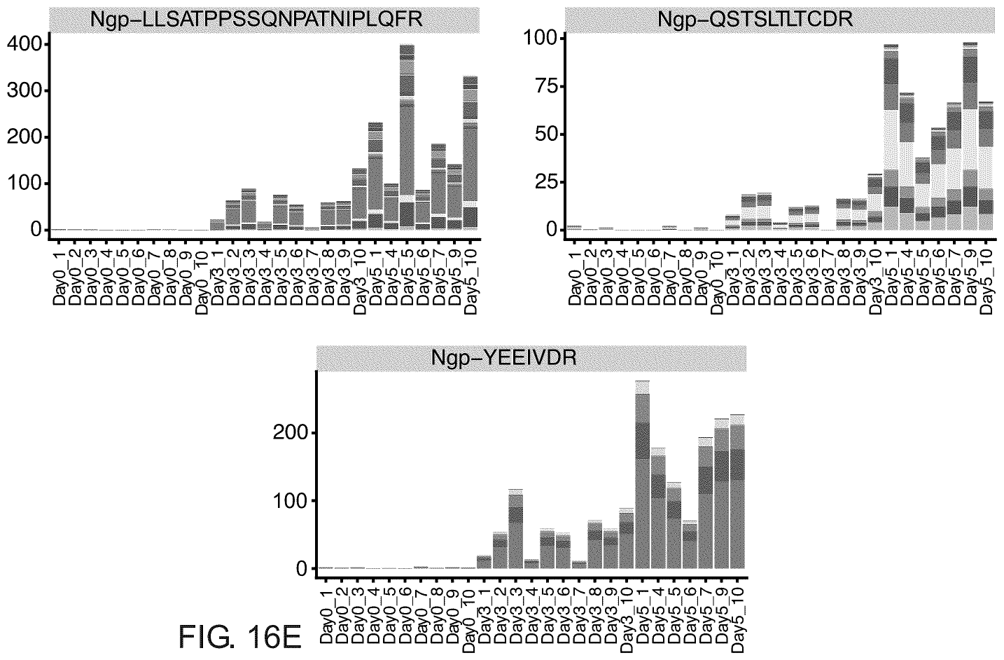

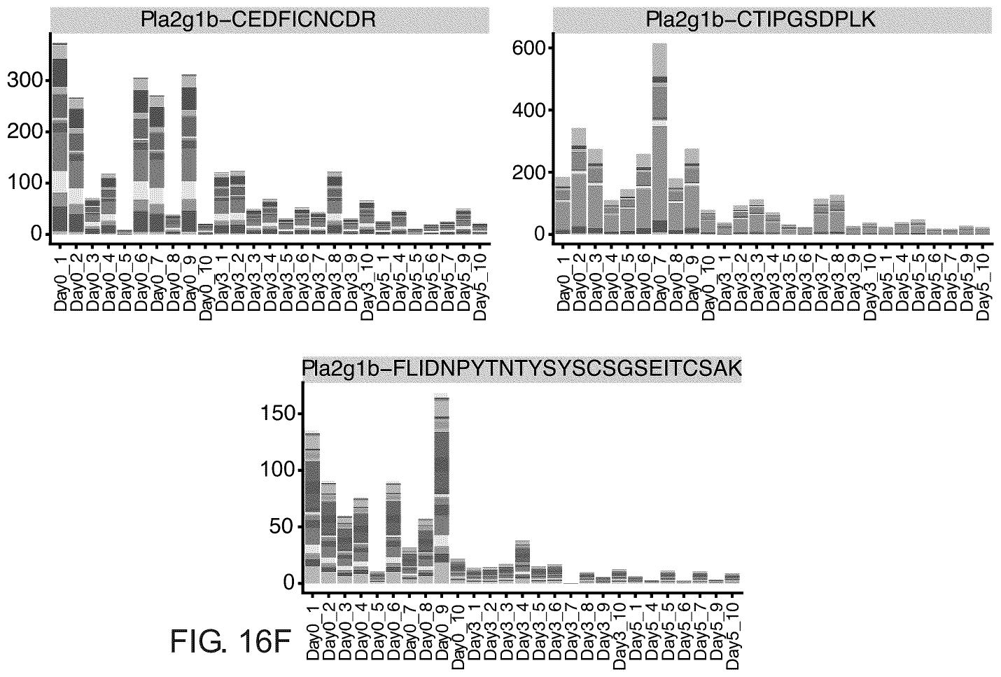

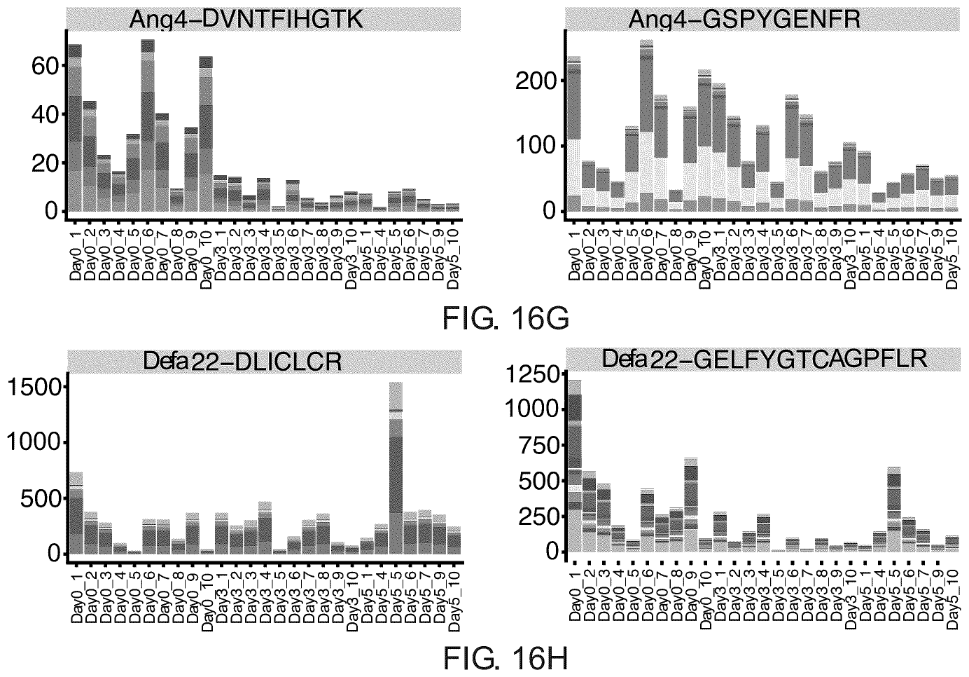

To verify the abovementioned results in the early inflammatory phases, an independent DSS colitis repeat experiment (FIG. 15A) was performed. Notably, the dynamics of the fecal AMP landscape (FIG. 15B-G) and microbiome configuration (FIG. 15H-J) were highly reproducible in this cohort. Importantly, the fecal AMP signature was highly correlated with both the individual-specific weight loss by linear regression analysis (R\hat{ }2=0.55, p=0.02, FIG. 15K) and the colitis-associated microbiome composition by Procrustes analysis (Correlation=0.91, p<0.001, FIG. 15L). Moreover, stool targeted proteomics focusing on 50 AMPs validated their changing patterns observed in the LFQ analysis (FIG. 16A-B, Table S3). A robust consistency in the changing patterns of different tryptic peptides corresponding to the same AMP was also observed, as exemplified by Camp, Lcn2, Ngp with increased abundance, and Pla2g1b, Ang4, Defa22 with decreased abundance along the early DSS course (FIG. 16C-H). Collectively, these data suggest that the dynamics of fecal AMP landscape upon induction of colonic inflammation are highly correlative of intestinal inflammatory clinical phases, and strongly associated with features of disease severity and dysbiosis.

Proteomic AMP signature predicts colonic inflammatory disease features. The significant correlations noted above between the proteomic AMP landscape and multiple microbiome and auto-inflammatory disease features prompted the investigation of the potential of fecal AMP profile to constitute a predictor of disease phenotypes, as a proof of concept of potential future use in disease diagnosis, prognostic assessment, and risk stratification. To this aim, both support vector machine (SVM) classification and gradient boosting regression machine learning algorithms were enlisted, as these may be capable of generating reliable predictions modelling complex non-linear features.

First, the proteomic fecal AMP predictive potential in assessing the stage of inflammation was evaluated in the murine DSS-induced model of intestinal inflammation, utilizing a multiclass SVM classifier with a linear kernel to distinguish between different phases of colonic inflammation (day 0, day 3, day 5, day 16 and day 31). Subsequently, SVM models were trained, applying a leave group out cross-validation scheme, leaving samples from one cage out in every training round and using it as the testing set, in order to handle the dependency between the samples in the same cage. Using this approach, an impressive performance of fecal AMP features in differentiating between baseline (day 0, accuracy 1.0), early pre-symptomatic phases (day 3 and day 5, accuracy 0.75 and 1.0, respectively), late inflammation during weight loss (day 16, accuracy 0.88) and the end of weight recovery phase (day 31, accuracy 0.75, FIG. 5A) was observed. Since alterations of fecal microbial composition uniquely accompanied the DSS colitis model (FIG. 4J-K) (64), the ability of fecal microbiome compositional features to predict disease phases was evaluated using the same approach. The results revealed that fecal microbiome features also performed well in classifying different phases of DSS-induced disease course (accuracy of 1.0, 0.88, 0.5, 0.88 and 0.88 for day 0, day 3, day 5, day 16 and day 31, respectively, FIG. 5B).

Next, the more difficult task of predicting an individual disease severity feature (weight loss) was assessed based on the fecal AMP signature, or alternatively by fecal microbiome features, and compared the predictive power of both. Indeed, disease severity of individual mice, as proxied by weight loss, could be readily predicted by the fecal AMP signature using the area under the curve (AUC) of weight loss over time as the target variable. To this aim, a gradient boosting regression algorithm applying the same leave one cage out cross-validation scheme was employed. A simple naïve baseline model was devised, estimating the AUC of weight loss from the mean of the training samples, and had a mean squared error (MSE) of 0.013. Notably, using the fold-changes of early-phase fecal AMP features (day 5 relative to day 0) to predict the individual-specific weight loss induced by DSS, a Pearson correlation coefficient of 0.64 between the predicted and observed weight loss with an MSE of 0.006 (FIG. 5C) was achieved. In contrast, the early-phase fecal microbiome alterations (day 5 compared to day 0) predicted the weight loss phenotype with a markedly inferior performance using the same algorithm (Pearson correlation coefficient=0.17, MSE=0.012, FIG. 5D). To account for the high dimensionality of the microbiome composition, a model with an additional pre-processing step of PCA was constructed, followed by a gradient boosting regression algorithm. Through this approach, the microbiome features performed significantly better but still inferior to the AMP features in predicting the weight loss (Pearson correlation coefficient=0.45, MSE=0.010, FIG. 5E), indicating that the fecal host AMP landscape constitutes a better predictor of intestinal inflammatory disease severity than the fecal microbiome configuration.

Given that the alterations in the AMP landscape may contribute to colitis-associated dysbiosis, the impact of changes in the abundance of individual AMPs on the abundance of specific bacterial species and functional pathways during acute colonic inflammation was investigated. Sparse partial least squares (sPLS) regression modeling was applied for the integration and selection of variables from different biological domains, attempting to predict the microbiome features with respect to fecal AMP signatures. Multiple strong pairwise associations (absolute threshold >0.7) were revealed between individual AMPs and both bacterial species (FIG. 5F) and pathways (FIG. 5G). Specifically, the decreased abundance of Reg1, BPI fold-containing family A member 2 (Bpifa2), and IEC-produced AMPs (Ang4, alpha-defensins), together with increased abundance of PMN-produced AMPs (Lcn2, Pglyrp2, Ltf, Ngp, Mpo, Camp) and other AMPs (Apcs, Pon1, Mb1, Ctsb, Vtn), was predictive of the loss of commensal bacterial species and the blooming of colitis-associated species (FIG. 5F). Changes of these AMPs were also highly predictive of a decreased abundance of fecal bacterial functional pathways related to the metabolism of cofactors and vitamins (ubiquinone and other terpenoid-quinone biosynthesis, lipoic acid metabolism) as well as increased abundance of multiple pathways related to carbohydrate metabolism, amino acid metabolism and lipid metabolism (FIG. 5G). Together, these results highlight that host fecal proteomic AMP signature is highly predictive of murine colitis-associated dysbiosis, as well as the resultant disease phases and severity.

The fecal proteomic AMP signature in human IBD. Finally, the AMP measurement pipeline was applied to human fecal samples. Human stool sample processing was similar to the abovementioned murine fecal sample processing, while the data were searched against the human protein database. Using this pipeline, the clinical utility of fecal AMP signature in differentiating Crohn's disease (CD) from healthy status was determined. To this end, 26 pediatric Crohn's disease (CD) patients and 28 age- and gender-matched healthy controls (Table S6) were recruited, and stool tandem discovery proteomics and shotgun metagenomics sequencing (FIG. 6A) was performed. In total, 403 proteins were detected by discovery proteomics from 3240 unique peptides. The non-invasive fecal global proteomic landscape in CD patients clustered differently from that of healthy controls (FIG. 17A, Table S3). Notably, AMPs comprised a significant signature among all the differentially abundant proteins (q<0.10, FIG. 6B), as confirmed by protein set enrichment analysis (FIG. 17B, Table S4). Indeed, the stool AMP landscape derived from the discovery proteomics featured clear separation between CD patients and healthy controls (FIG. 6C, Table S3). Importantly, 16 out of the 49 identified AMPs were significantly increased in the stool samples of CD patients, including the PMN-produced Prtn3, Azul, Ltf, Ctsg, Pglyrp1, Mpo, Elane, S100a8, S100a9, S100a12 (FIG. 6D). In contrast, the IEC-originated AMPs Lgals4 and Pla2g 1b were significantly decreased in CD patients (FIG. 6D). This phenomenon recapitulates the observations from the murine DSS model (FIG. 4G-H), and indicates a significantly altered AMP landscape in the stool of CD patients compared to healthy controls which is more diverse that the clinically measured calprotectin.

Shotgun metagenomics sequencing of stool samples collected from the same participants revealed similar patterns of variation in the microbial taxonomic profiles that largely separated CD patients from healthy controls (FIG. 6E, Table S3). In line with previous findings (Franzosa et al., 2019), the fecal microbial configuration in CD patients featured significantly decreased alpha-diversity compared to healthy controls, as quantified by Shannon diversity (FIG. 17C). Consistently, of the bacterial species that were differentially abundant in CD samples (q<0.10), species including Dialister. sp, Alistipes obesi, Senegalimassilia anaerobia, Sutterella parvirubra, Massilimallia timonesis and Bifidobacterium spp were depleted in CD, while species Anearostipes caccae, Eggthella lenta and Erysipelatoclostridium ramosum were enriched in CD (FIG. 6F).

To identify the association between fecal AMP and microbiome, Procrustes analysis was performed by integrating the proteomic and metagenomic datasets. Notably, the fecal AMP landscape was significantly correlated with microbial community composition (FIG. 6G, correlation=0.63, p<0.001), highlighting a strong coupling of intestinal AMP signature and the microbial taxonomy profile. By applying sPLS regression modeling, multiple potential mechanistic associations between CD-linked individual AMPs and microbes were identified (absolute threshold >0.35, FIG. 6H). Interestingly, the increased abundance of AMPs mainly produced by PMN (e.g., Prtn3, Azul, Ltf, Ctsg, S100a8, S100a9, S100a12), along with the decreased abundance of AMPs including Lgals4, Gp2, Asah2, was associated with the depletion of bacterial species (Prevotella spps, Bifidobacterium adolescentis, Senegalimassilia anaerobia) and enrichment of colitis-associated bacteria in CD patients (e.g., Eggerthella lenta, Escherichia spps, Citrobacter braakii and Clostridioides difficile) (FIG. 6H).

To evaluate if alterations in the fecal AMP or microbial composition could be utilized to differentiate CD versus healthy status, sigmoid kernel SVM classifiers were trained on the AMP and bacterial species profiles, both separately and combined, using a stratified K-fold cross-validation (K=5) to avoid gender bias. SVM classifiers trained on fecal AMP features (mean AUC 0.86) versus microbial species (mean AUC 0.86) performed similarly well (FIG. 6I). Furthermore, incorporation of AMPs and bacterial species resulted in slightly better classification accuracy (mean AUC 0.89) relative to AMP features alone (FIG. 6I), likely stemming from the strong association between the intestinal AMP and microbiome signatures.

To further identify potential AMP signatures that are associated with disease severity, the CD patients were stratified into active and inactive disease according to the pediatric Crohn's disease activity index (PCDAI) scores collected upon recruitment (FIG. 6A, Table S6).

TABLE S6

| | Healthy Controls | Crohn's Disease | |
|---|---|---|---|
| | (n = 28) | (n = 26) | P value |
| Mean age (SEM) | 13.53 (0.56) | 13.96 (0.64) | 0.62 |
| Male:Female ratio | 13:15 | 18:8 | 0.11 |

| | Inactive Disease | Active Disease | |
|---|---|---|---|
| | (PCDAI = 0, n = 15) | (PCDAI > 0, n = 11) | |
| Mean age (SEM) | 13.69 (0.88) | 14.31 (0.97) | 0.65 |
| Male:Female ratio | 11:4 | 7:4 | 0.68 |

Based on PCA, a clearer separation of the AMP profile was observed between active and inactive disease status than the microbiome features, although neither was significant, likely due to low sample size (FIG. 17D-E, Table S3). Notably, AMP remains a significant signature among differentially abundant proteins (p<0.10) between patients with active and inactive disease status (FIG. 17F, Table S4). Besides the clinically used AMP calprotectin (S100a9 and S100a8), multiple other AMPs including Apcs, Mpo, Prtn3, Ltf, Pglyrp1 and Elane also featured significantly higher abundance in CD patients with active disease manifestations compared to inactive status (FIG. 17G). Taken together, these results provide insight into the microbiome-host AMP interactome in human IBD, and highlight the clinically important potential of the non-invasive stool AMP signature of the invention in accurately differentiating, clinically stratifying and assessing treatment responsiveness in human IBD.

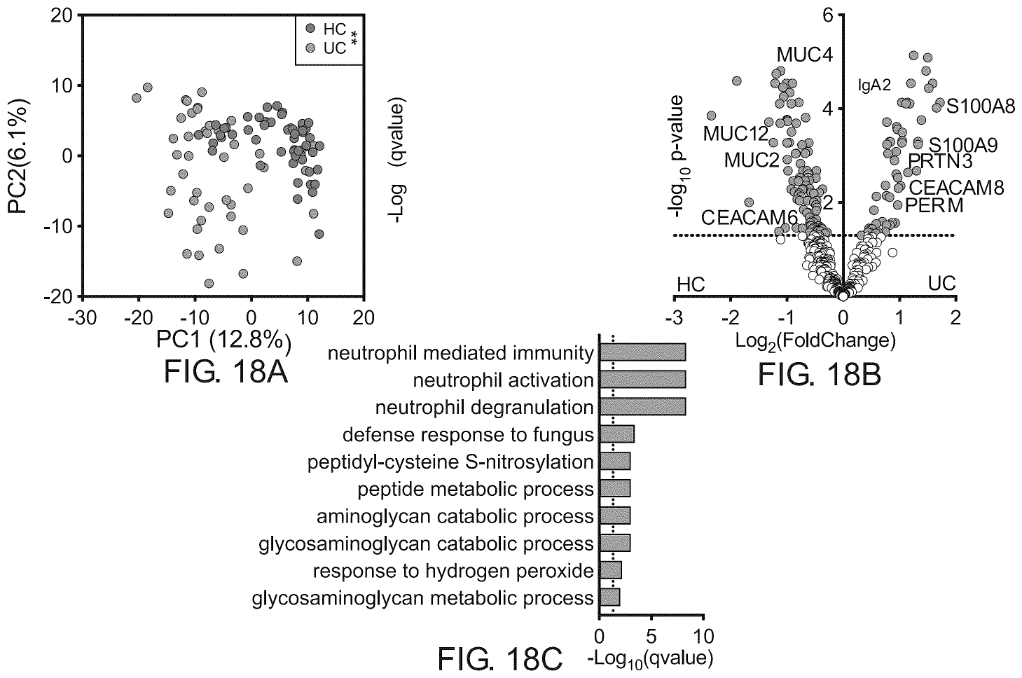

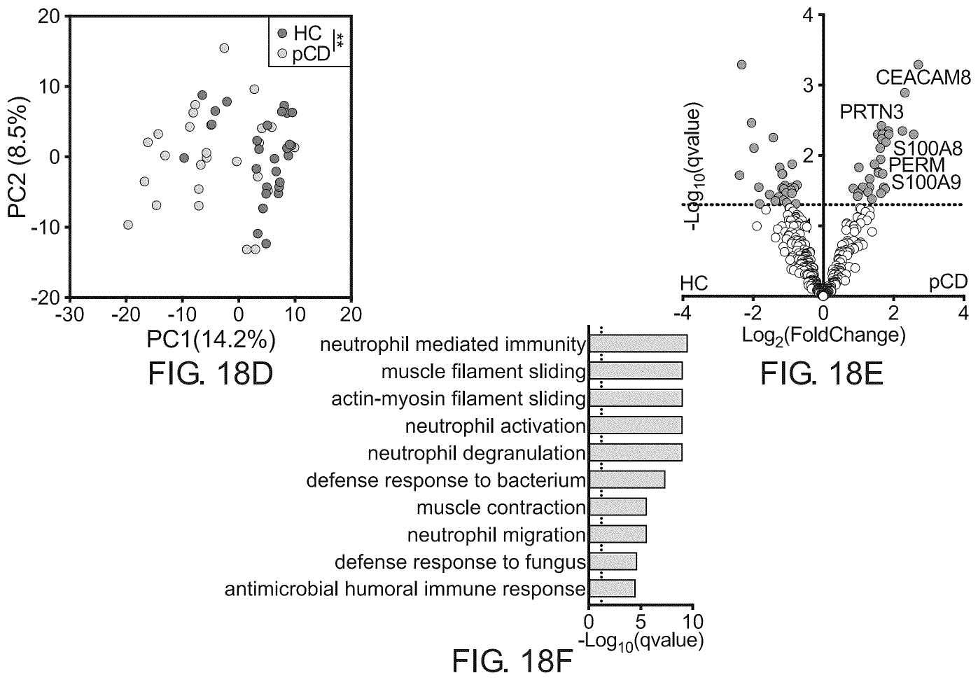

Further investigation into fecal AMP signatures in IBD revealed significant modifications of AMPs in fecal samples from large cohorts of Ulcerative Colitis and Crohn's Disease patients, compared to those from healthy control patients. FIGS. 18A-18C are graphic representations of significant alterations in the proteomes of the Ulcerative Colitis subjects, compared to the healthy controls. FIG. 18A shows that the two groups are significantly distinct in the distribution of the altered proteins/peptides detected. FIG. 18B represents those proteins/peptides identified having significant (cut-off is above the qvalue indicated by the dotted line) fold change (positive or negative) relative to the healthy control samples. Clearly significant changes can be identified in AMP proteins/peptides (green dots) such as MUC2, MUC4, MUC12, S100A8, S100A9, CEACAM8, PERM, PRTN3 and IgA2, among others (not named). In FIG. 18C, proteins/peptides having significantly altered (high q value, see FIG. 18B) representation in fecal samples from UC patients are grouped according to their biological/physiological function. It will be appreciated that the most significantly altered proteins/peptides in UC fall into four immune-related categories (neutrophil mediated immunity, neutrophil activation, neutrophil degranulation and defense response to fungus), indicating antimicrobial peptide/protein character.

FIGS. 18D-18F are graphic representations of significant alterations in the proteomes of the Crohn's Disease (CD) subjects, compared to the healthy controls (HC). FIG. 18D shows that the two groups are significantly distinct in the distribution of the altered proteins/peptides detected. FIG. 18E represents those proteins/peptides identified having significant (cut-off is above the q value indicated by the dotted line) fold change (positive or negative) relative to the healthy control samples. Clearly significant changes can be identified in AMP proteins/peptides (green dots) such as S100A8, S100A9, CEACAM8, PERM and PRTN3, among others (not named). In FIG. 18F, proteins/peptides having significantly altered (high qvalue, see FIG. 18E) representation in fecal samples from CD patients are grouped according to their biological/physiological function. It will be appreciated that nearly all of the significantly altered proteins/peptides in CD fall into six immune-related categories (neutrophil mediated immunity, neutrophil activation, neutrophil degranulation and defense response to bacterium, neutrophil migration, defense response to bacterium and antimicrobial humoral immune response), indicating antimicrobial peptide/protein character.

Proteomic AMP signature can be predictive in Primary Sclerosing Cholangitis. Primary Sclerosing Cholangitis is a disease of the bile ducts. In primary sclerosing cholangitis, inflammation causes scars within the bile ducts, gradually causing serious liver damage. A majority of people with primary sclerosing cholangitis also have inflammatory bowel disease.

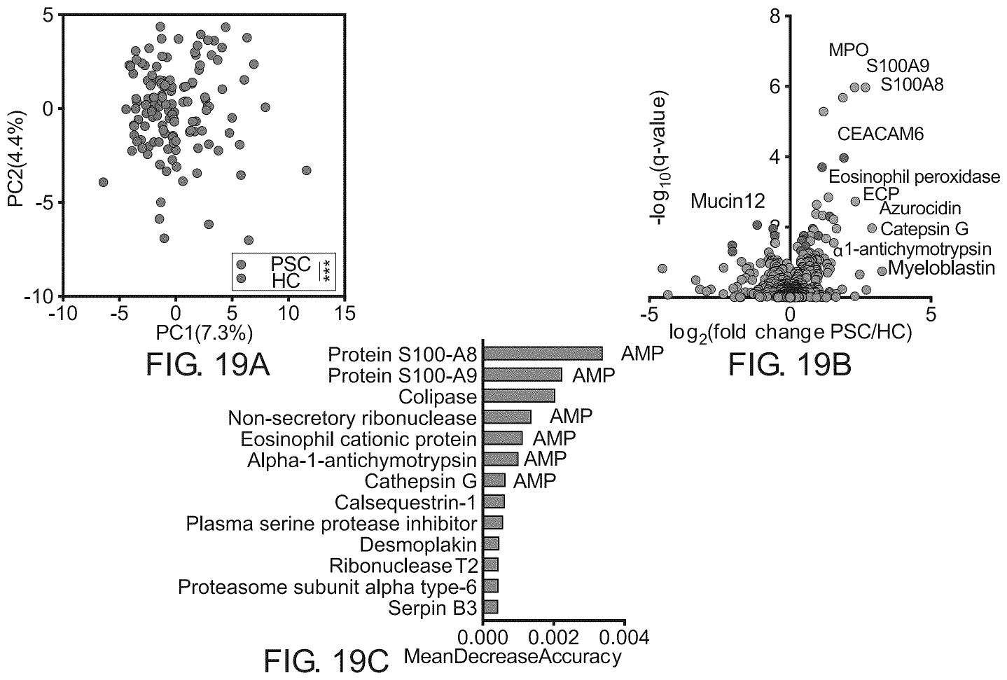

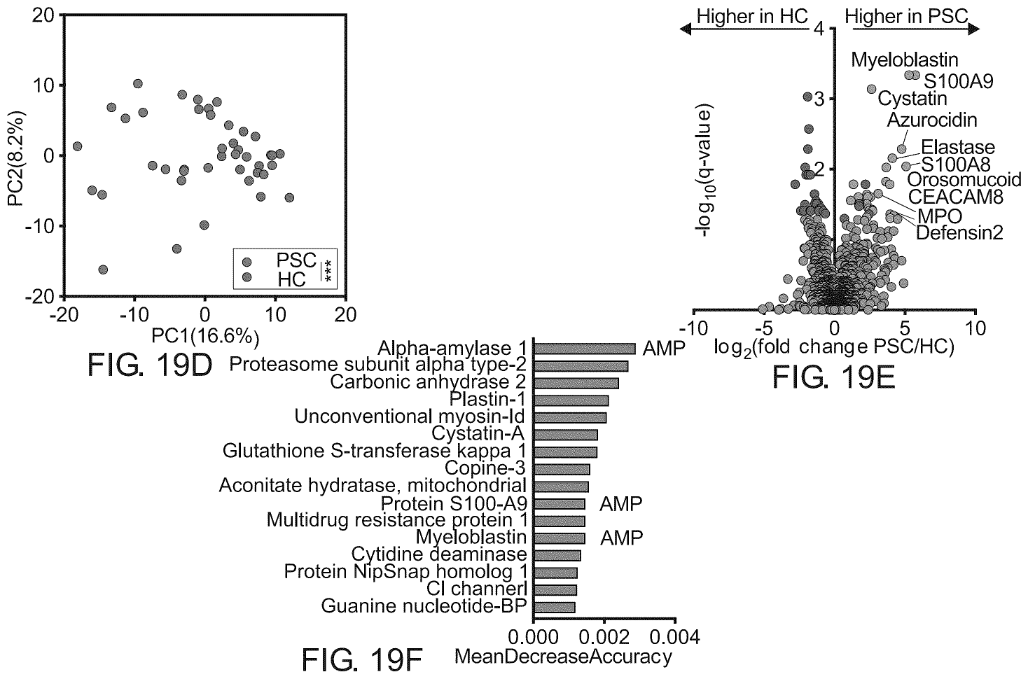

Investigation into fecal AMP signatures in Primary Sclerosing Cholangitis (PSC) revealed significant modifications of AMPs in fecal samples from large cohorts of PSC patients, compared to those from healthy control patients. FIGS. 19A-19C are a graphic representations of significant alterations in the proteomes of the PSC subjects, compared to the healthy controls. FIG. 19A is a plot of the principal component analysis of a large cohort (n=65) of German patients and matched HC, showing that stool proteomic signature of the two groups is significantly distinct in the distribution of the altered proteins/peptides detected. FIG. 19B represents those proteins/peptides identified having significant (cut-off is above the qvalue indicated by the dotted line) fold change (positive or negative) relative to the healthy control samples. Clearly significant changes can be identified in AMP proteins/peptides (green dots) such as MUC12, S100A8, S100A9, CEACAM8, Eosinophil peroxidase (ECP), Azurocidin (AZU), Catepsin G (CTSG), Myeloperoxidase (MPO, PERM), alpha-1-antichymotrypsin and Myeloblastin (PRTN3), among others (not named). FIG. 19C ranks proteins/peptides identified as most significant discriminating features for distinguishing between fecal samples from PSC patients and HC, based on a machine learning algorithm. AMPs (S100-A8, S100-A9, Non-secretory RNase (RNAS4), Eosinophil cationic protein (ECP2), Alpha-1-antichymotrypsin and Cathepsin G(CTSG)) are among the most important disease-discriminatory features.

FIGS. 19D-19F represent a similar analysis of PSC patients from Israel (n=17, 25 matched HC). Note that the fecal AMP profile of the Israeli PSC patients (FIG. 19E) is similar to that of the German cohort (18B)-Myeloblastin (PRTN3), S100-A9, S100-A8, Cystatin (CSTA, CYTA), Azurocidin (AZU), Elastase (ELANE), Orosomucoid, CEACAM8, MPO (PERM) and Defensin (Defa). Likewise, AMPs (Alpha-amylase 1, S100 A9, Myeloblastin(PRTN3)) were identified as important disease-discriminatory features of Israeli PSC patients by the machine learning algorithm (FIG. 19F).

Table 7 below shows the results of the IBD and PSC AMP profiles from the clinical data.

TABLE S7
HUMAN AMP Profiles- Clinical Data

| AMP Signature | Significantly | |
|---|---|---|
| in comparisons | decreased AMPs | Significantly increased AMPs |
| UC vs Healthy | “MUC2”, | “S100A8”, “CEACAM8”, “Perm”, |
| | “MUC4”, | “Prtn3”, “IgA” |
| CD vs Healthy | “MUC12”, | “S100A8”, “S100A9”, |
| | “CEACAM6” | “CEACAM8”, “Perm”, “Prtn3” |
| PSC vs Healthy | “MUC12” | S100A8, S100A9, CEACAM8, |
| | | “Ecp2”, “Azu”, “Ctsg”, “Perm”, |
| | | alpha-1 antichymotrypsin, “Prtn3”, |
| | | “Exp”, “RnaS4”, “Elane”, “CstA”, |
| | | Orosomucoid, alpha amylase 1 |

AMPs identified in the clinical results detailed in Table S7, their gene name and their Uniprot Accession number include:

TABLE S8

| | | UniProt |
|---|---|---|
| Protein name | Gene name | Access No. |
| Alpha Amylase | AmylA | PODUB6 |
| Alpha-1-antichymotrypsin | AACT, | P01011 |
| | SERPINA3 | |
| Azurocidin | Azu | P20160 |
| Alpha-1 Acid Glycoprotein | Orm1, | P02763 |
| (Orosomucoid) | A1AG1 | |
| Eosinophil Cationic Protein | Ear2 | P97425 |
| Neutrophil Elastase | Elane | Q3UP87 |
| Non-Secretory RNase | Rnas4 | Q9JJHI |
| Eosinophil Peroxidase | Epx | P49290 |
| Cystatin-A; Cystatin-A, N- | Csta | P56567 |
| terminally processed | | |
| Cathepsin G | Ctsg | P28293 |
| Mucin2 | Muc2 | Q02817 |
| Mucin 4 | Muc4 | Q99102 |
| Mucin 12 | Muc12 | Q9UKN1 |
| Myeloblastin | Prtn3 | Q61096 |
| Myeloperoxidase | Perm | P11247 |
| Protein S100-A8 | S100a8 | P27005 |
| Protein S100-A9 | S100a9 | P31725 |

### DISCUSSION

The murine intestinal AMP proteomic landscape in steady-state and intestinal perturbation was characterize in protein-level detail, demonstrating the superiority of protein-level over transcriptomic AMP assessment, and highlighting the efficacy of establishing a lower GI landscape by stool AMP profiling. Utilizing correlational and predictive machine learning-based analysis, it was shown that that the host AMP landscape could accurately predict microbiome dynamics and disease severity features in murine intestinal disorders. Finally, a similar proteomic AMP-based pipeline was developed for humans, demonstrating that non-invasive fecal AMP features can enable accurate differentiation and classification between human IBD and healthy status.

These data have several potentially important implications. First, they highlight that, in contrast to previous paradigms, context-specific AMP dynamics are marked by global protein-level signature shifts, rather than single AMP alterations, or those proxied by their mRNA levels. As such, the proteomic pipeline of the invention not only accurately validates previously reported individual mRNA AMP changes, it importantly demonstrates that these occur within a much larger framework of highly regulated AMP signature dynamics. Such population-level AMP elucidation also enabled the identification of several new AMP candidates, based on structure and protein level dynamics similarities to known AMPs. For example, Retnlg (structurally similar to other resistin-like AMPs Retnlb and Retnla, with increased abundance in C. rodentium infection and DSS colitis), Mptx 1 (belonging to the pentraxins family, with increased abundance in SFB colonization and decreased abundance in C. rodentium infection and DSS-colitis), and Chil3 (a bacteria-binding lectin with increased abundance in SFB colonization, C. rodentium infection and DSS colitis) were identified by the pipeline of the invention as new AMP candidates.

Second, such context-specific global AMP dynamics enable an expanded insight into host regulation of the interactions with its microbiome in homeostasis and disease. For example, during commensal mono- or poly-inoculation, IECs dominate in their AMP secretion responsiveness, possibly in selecting bacterial colonizers in specific intestinal niches, and in shaping host immune responses to diverse commensal organisms. As such, commensal mSFB adhesion and colonization in the murine ileum can leads to secretion of an array of IEC-produced AMPs, which, in turn, are strongly associated with the mucosal mSFB abundance.

Intriguingly, infection by enteric bacterial pathogens elicits a significant suppression of IEC-produced AMPs (e.g., Reg4, Ang4), possibly representing an evolved pathogen-mediated exploitation of the invaded host, in facilitating its colonization while outcompeting commensals. The host's response in this ‘arms race’ includes a massive induction of AMPs originating from recruited immune cells, which are highly positively associated with mucosal-attached or fecal C. rodentium abundance, therefore likely aimed at limiting pathogen expansion. In contrast, a chemical inflammatory insult with a primary perturbation at the IEC level leads to a massive and improper approximation of the intestinal microbiome and the mucosal immune system. This results in a massive alteration in the secreted AMP landscape, which closely reflects (and predicts) this compositional and functional dysbiotic state. Consequently, this inflammatory AMP signature is characterized by a significant decrease in IEC-produced AMPs, coupled with a profound increase of PMN-secreted AMPs, ECM-associated AMPs and lipoproteins collectively driving a de-novo bloom of colitis-associated bacteria, inflammation and tissue damage. This inflammatory phase is followed by, and inter-connected with wound healing and tissue repair aimed at curbing the inflammation and correcting tissue damage. Intriguingly, in the recovery phase, reconstitution of the IEC layer and part of the IEC-associated AMPs, coupled with the persistence of infiltrating immune cells and their respective AMPs, leads to a peculiar state marked by a mixed immune-IEC AMP repertoire. The methods of the present invention can contribute to disentangling the complex relationships between the roles of specific AMP subsets in contributing to host-microbiome crosstalk and associated clinical symptoms in auto-inflammatory diseases such as IBD, and recovery from disease exacerbation in these disorders.

Finally, the results highlight the significance of the potential utilization of host fecal AMP signatures in predicting microbiome dynamics and disease outcomes. Indeed, and in contrast to the labile AMP mRNA, a high consistency between the fecal and mucosal protein AMP landscapes was established, highlighting their potential to non-invasively proxy mucosal AMP features. In steady-state, patient-specific AMP repertoires may complement the fecal microbiome in generating individualized signatures. During disease, such AMP signatures and their dynamics may precede and predict disease exacerbation, even before the development of clinical symptoms, while acting as accurate predictors of individualized disease severity, propensity for complications, and treatment responsiveness. Indeed, fecal calprotectin (S100a9/S100a8 complex) has been shown to correlate with IBD disease severity. Even more ambitiously, mechanistic understanding of global AMP functions in the gastrointestinal niche may enable deployment of them as microbiome modulators, as part of AMP-targeted treatment.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of determining presence of Inflammatory Bowel Disease (IBD) in the gut mucosa of a subject, the method comprising measuring levels of azurocidin (AZU) in a stool sample from said subject, wherein increased levels of AZU in said stool sample compared to normal levels is indicative of IBD.

2. The method of claim 1, wherein said increased levels of AZU in said stool sample is levels greater than that in stool sample of a subject free of IBD.

3. The method of claim 1, wherein said determining said levels of AZU is effected on a protein extract of said stool sample.

4. The method of claim 1, wherein said stool sample is depleted of membrane proteins and membrane-bound organelle proteins.

5. The method of claim 1, wherein said subject is suffering from IBD symptoms selected from the group consisting of weight loss, bloody diarrhea, destruction of epithelial cells, and dysregulation of mucosal immune responses.

6. The method of claim 1, wherein said AZU is identified by liquid chromatography, mass spectrometry or a combination of liquid chromatography and mass spectrometry.

7. The method of claim 1, wherein said AZU is identified by mass spectrometry-based label free quantification (LFQ).

8. The method of claim 1, wherein said AZU is identified by AMP binding moieties.

9. The method of claim 8, wherein said AMP binding moieties are immobilized on an array.

10. A kit for determining the presence of IBD in the gut, comprising at least one AZU-binding moiety.

11. The kit of claim 10, wherein said at least one binding moiety is selected from the group consisting of antibodies, aptamers and ligands.

12. A method of treating IBD in a subject suspected of having IBD, comprising:
(i) measuring the level of AZU in a stool sample of said subject;
(ii) determining the presence of IBD in the gut of the subject based on the level of AZU in said stool sample being greater than the level of AZU in a stool sample of a normal healthy subject; and
(iii) treating said IBD in said subject confirmed as having IBD.

13. A method of monitoring response of a subject to treatment for IBD, comprising:
(i) determining the presence of IBD in the gut of the subject by measuring the level of AZU in a stool sample of said subject,
(ii) initiating treatment for IBD,
(iii) measuring the level of AZU in a second stool sample of said subject at a predetermined interval from step (ii),
(iv) comparing the levels of AZU from step (i) and step (iii), and
(v) determining the response of the subject according to the difference in AZU levels in the first and second stool samples.

14. The method of claim 13, further comprising adjusting said treatment of said subject for IBD according to the response monitored in step (v).

15. The method of claim 13, wherein the presence of IBD in the gut of said subject is determined based on the level of AZU in said stool sample being greater than the level of AZU in a stool sample of a normal healthy subject.

16. The method of claim 13, further comprising additional measurements of AZU in at least one additional stool sample at at least one predetermined interval from said second stool sample;
comparing the levels of AZU with those from previous levels; and
determining the response of the subject according to the difference in AZU levels in the previous and additional stool samples.


SEQUENCE LISTING (47 sequences)

SEQ ID NO: 1 - DNA, 60 bp, synthetic construct
  misc_feature 1..60: note = Single strand DNA oligonucleotide
  source 1..60: mol_type = other DNA
aatgatacggcgaccaccgagatctacactatggtaattgtgtgccagcmgccgcggtaa

SEQ ID NO: 2 - DNA, 68 bp, synthetic construct
  misc_feature 1..68: note = Single strand DNA oligonucleotide
  misc_feature 25..36: note = n is a, c, g, or t
  source 1..68: mol_type = other DNA
caagcagaagacggcatacgagatnnnnnnnnnnnnagtcagtcagccggactachvgggtwtctaat

SEQ ID NO: 3 - protein, 410 aa, Mus musculus
MKTPGVLLLILGLLASSSFAIIRIPLRKFTSIRRTMTEVGGSVEDLILKGPITKYSMQSSPKTTEPVSELLKNYLDAQYYGDIGIGTPPQCFTVVFDTGSSNLWVPSIHCKILDIACWVHHKYNSDKSSTYVKNGTSFDIHYGSGSLSGYLSQDTVSVPCKSDQSKARGIKVEKQIFGEATKQPGIVFVAAKFDGILGMGYPHISVNNVLPVFDNLMQQKLVDKNIFSFYLNRDPEGQPGGELMLGGTDSKYYHGELSYLNVTRKAYWQVHMDQLEVGNELTLCKGGCEAIVDTGTSLLVGPVEEVKELQKAIGAVPLIQGEYMIPCEKVSSLPTVYLKLGGKNYELHPDKYILKVSQGGKTICLSGFMGMDIPPPSGPLWILGDVFIGSYYTVFDRDNNRVGFANAVVL

SEQ ID NO: 4 - protein, 334 aa, Mus musculus
MNLLLLLAVLCLGTALATPKFDQTFSAEWHQWKSTHRRLYGTNEEEWRRAIWEKNMRMIQLHNGEYSNGQHGFSMEMNAFGDMTNEEFRQVVNGYRHQKHKKGRLFQEPLMLKIPKSVDWREKGCVTPVKNQGQCGSCWAFSASGCLEGQMFLKTGKLISLSEQNLVDCSHAQGNQGCNGGLMDFAFQYIKENGGLDSEESYPYEAKDGSCKYRAEFAVANDTGFVDIPQQEKALMKAVATVGPISVAMDASHPSLQFYSSGIYYEPNCSSKNLDHGVLLVGYGYEGTDSNKNKYWLVKNSWGSEWGMEGYIKIAKDRDNHCGLATAASYPVVN

SEQ ID NO: 5 - protein, 1153 aa, Mus musculus
MTLKALLVTALALCHGFNLDTEHPMTFQENAKGFGQNVVQLGGTSVVVAAPQEAKAVNQTGALYQCDYSTSRCHPIPLQVPPEAVNMSLGLSLAVSTVPQQLLACGPTVHQNCKENTYVNGLCYLFGSNLLRPPQQFPEALRECPQQESDIVFLIDGSGSINNIDFQKMKEFVSTVMEQFKKSKTLFSLMQYSDEFRIHFTFNDFKRNPSPRSHVSPIKQLNGRTKTASGIRKVVRELFHKTNGARENAAKILVVITDGEKFGDPLDYKDVIPEADRAGVIRYVIGVGNAFNKPQSRRELDTIASKPAGEHVFQVDNFEALNTIQNQLQEKIFAIEGTQTGSTSSFEHEMSQEGFSASITSNGPLLGSVGSFDWAGGAFLYTSKDKVTFINTTRVDSDMNDAYLGYASAVILRNRVQSLVLGAPRYQHIGLVVMFRENFGTWEPHTSIKGSQIGSYFGASLCSVDMDADGNTNLILIGAPHYYEKTRGGQVSVCPLPRGRARWQCEALLHGDQGHPWGRFGAALTVLGDVNGDKLTDVAIGAPGEQENQGAVYIFYGASIASLSASHSHRIIGAHFSPGLQYFGQSLSGGKDLTMDGLMDLAVGAQGHLLLLRAQPVLRLEATMEFSPKKVARSVFACQEQVLKNKDAGEVRVCLRVRKNTKDRLREGDIQSTVTYDLALDPVRSRIRAFFDETKNNTRRRTQVFGLMQKCETLKLILPDCVDDSVSPIILRLNYTLVGEPLRSFGNLRPVLAMDAQRFFTAMFPFEKNCGNDSICQDDLSITMSAMGLDTLVVGGPQDFNMSVTLRNDGEDSYGTQVTVYYPSGLSYRKDSASQNPLTKKPWFVKPAESSSSSEGHGALKSTTWNINHPIFPANSEVTFNVTFDVDSHASFGNKLLLKAIVASENNMSRTHKTKFQLELPVKYAIYMIVTSDESSIRYLNFTASEMTSKVIQHQYQFNNLGQRSLPVSVVFWIPVQINNVTVWDHPQVIFSQNLSSACHTEQKSPPHSNFRDQLERTPVLNCSVAVCKRIQCDLPSFNTQEIFNVTLKGNLSFDWYIKTSHGHLLLVSSTEILFNDSAFALLPGQESYVRSKTETKVEPYEVHNPVPLIVGSSIGGLVLLALITAGLYKLGFFKRQYKDMMNEAAPQDAPPQ

SEQ ID NO: 6 - protein, 448 aa, Mus musculus
MKILLLCVALLLIWDNGMVLGEQEVSDNELQELSTQGSRYINKEIQNAVQGVKHIKTLIEKTNAERKSLLNSLEEAKKKKEDALEDTRDSEMKLKAFPEVCNETMMALWEECKPCLKHTCMKFYARVCRSGSGLVGQQLEEFLNQSSPFYFWMNGDRIDSLLESDRQQSQVLDAMQDSFARASGIIDTLFQDRFFARELHDPHYFSPIGFPHKRPHFLYPKSRLVRSLMSPSHYGPPSFHNMFQPFFEMIHQAQQAMDVQLHSPAFQFPDVDFLREGEDDRTVCKEIRRNSTGCLKMKGQCEKCQEILSVDCSTNNPAQANLRQELNDSLQVAERLTEQYKELLQSFQSKMLNTSSLLEQLNDQFNWVSQLANLTQGEDKYYLRVSTVTTHSSDSEVPSRVTEVVVKLFDSDPITVVLPEEVSKDNPKFMDTVAEKALQEYRRKSRAE

SEQ ID NO: 7 - protein, 165 aa, Mus musculus
MARNAYFILLSCLIVLSPSQGQEAEEDLPSARISCPEGSNAYSSYCYYFTEDRLTWADADLFCQNMNSGYLVSVLSQAEGNFVASLIKESGTTDANVWTGLHDPKRNRRWHWSSGSLFLYKSWATGSPNSSNRGYCVSLTSNTGYKKWKDDNCDAQYSFVCKFKG

SEQ ID NO: 8 - protein, 173 aa, Mus musculus
MAQNNVYLILFLCLMFLSYSQGQVAEEDFPLAEKDLPSAKINCPEGANAYGSYCYYLIEDRLTWGEADLFCQNMNAGHLVSILSQAESNFVASLVKESGTTASNVWTGLHDPKSNRRWHWSSGSLFLFKSWATGAPSTANRGYCVSLTSNTAYKKWKDENCEAQYSFVCKFRA

SEQ ID NO: 9 - protein, 202 aa, Mus musculus
MGFWGTYLLFCLFSFLSQLTAESPTPKAKKAANAKKDLVSSKMFEELKNRMDVLAQEVALLKEKQALQTVCLKGTKVNLKCLLAFTQPKTFHEASEDCISQGGTLGTPQSELENEALFEYARHSVGNDANIWLGLNDMAAEGAWVDMTGGLLAYKNWETEITTQPDGGKAENCAALSGAANGKWFDKRCRDQLPYICQFAIV

SEQ ID NO: 10 - protein, 98 aa, Mus musculus
MMCGAPSATMPATAETQEVADQVKSQLESKENQKFDVFKAISFKRQIVAGTNLFIKVDVGGDKCVHLRVFQPLPHENKPLTLSSYQTNKERHDELSYF

SEQ ID NO: 11 - protein, 771 aa, Mus musculus
MLGLRPSLLLALAGLFFLGSAVSQECTKYKVSSCRDCIQSGPGCSWCQKLNFTGPGEPDSLRCDTRAQLLLKGCPADDIMDPRSIANPEFDQRGQRKQLSPQKVTLYLRPGQAAAFNVTFRRAKGYPIDLYYLMDLSYSMLDDLNNVKKLGGDLLQALNEITESGRIGFGSFVDKTVLPFVNTHPEKLRNPCPNKEKACQPPFAFRHVLKLTDNSNQFQTEVGKQLISGNLDAPEGGLDAIMQVAACPEEIGWRNVTRLLVFATDDGFHFAGDGKLGAILTPNDGRCHLEDNMYKRSNEFDYPSVGQLAHKLSESNIQPIFAVTKKMVKTYEKLTEIIPKSAVGELSDDSSNVVQLIKNAYYKLSSRVFLDHSTLPDTLKVTYDSFCSNGASSIGKSRGDCDGVQINNPVTFQVKVMASECIQEQSFVIRALGFTDTVTVQVRPQCECQCRDQSREQSLCGGKGVMECGICRCESGYIGKNCECQTQGRSSQELERNCRKDNSSIVCSGLGDCICGQCVCHTSDVPNKEIFGQYCECDNVNCERYNSQVCGGSDRGSCNCGKCSCKPGYEGSACQCQRSTTGCLNARLVECSGRGHCQCNRCICDEGYQPPMCEDCPSCGSHCRDNHTSCAECLKFDKGPFEKNCSVQCAGMTLQTIPLKKKPCKERDSEGCWITYTLQQKDGRNIYNIHVEDSLECVKGPNVAAIVGGTVVGVVLIGVLLLVIWKALTHLTDLREYRRFEKEKLKSQWNNDNPLFKSATTTVMNPKFAES

SEQ ID NO: 12 - protein, 97 aa, Mus musculus
MIPGGLTEARPATAEVQEIADRVKAQLEEETNEKYEIFKAVEYKTQVVAGVNYFIKMDVGGGCFTHIKVFKDLSGKNNLELTGYQTNKTEDDELTYF

SEQ ID NO: 13 - protein, 179 aa, Mus musculus
MWATRGLAVALALSVLPDSRALRPGDCEVCISYLGRFYQDLKDRDVTFSPATIEEELIKFCREARGKENRLCYYIGATDDAATKIINEVSKPLAHHIPVEKICEKLKKKDSQICELKYDNQIDLSTVDLKKLRVKELKKILDDWGEMCKGCAEKSDYIRKINELMPKYAPKAASARTDL

SEQ ID NO: 14 - protein, 412 aa, Mus musculus
MDRHLCTCRETQLRSGLLLPLFLLMMLADLTLPAQRHPPVVLVPGDLGNQLEAKLDKPKVVHYLCSKKTDSYFTLWLNLELLLPVIIDCWIDNIRLVYNRTSRATQFPDGVDVRVPGFGETFSMEFLDPSKRNVGSYFYTMVESLVGWGYTRGEDVRGAPYDWRRAPNENGPYFLALREMIEEMYQMYGGPVVLVAHSMGNVYMLYFLQRQPQVWKDKYIHAFVSLGAPWGGVAKTLRVLASGDNNRIPVIGPLKIREQQRSAVSTSWLLPYNHTWSHEKVFVYTPTTNYTLRDYHRFFRDIGFEDGWFMRQDTEGLVEAMTPPGVELHCLYGTGVPTPNSFYYESFPDRDPKICFGDGDGTVNLESVLQCQAWQSRQEHRVSLQELPGSEHIEMLANATTLAYLKRVLLEP

SEQ ID NO: 15 - protein, 398 aa, Mus musculus
MAKLILVTGLAILLNVQLGSSYQLMCYYTSWAKDRPIEGSFKPGNIDPCLCTHLIYAFAGMQNNEITYTHEQDLRDYEALNGLKDKNTELKTLLAIGGWKFGPAPFSAMVSTPQNRQIFIQSVIRFLRQYNFDGLNLDWQYPGSRGSPPKDKHLFSVLVKEMRKAFEEESVEKDIPRLLLTSTGAGIIDVIKSGYKIPELSQSLDYIQVMTYDLHDPKDGYTGENSPLYKSPYDIGKSADLNVDSIISYWKDHGAASEKLIVGFPAYGHTFILSDPSKTGIGAPTISTGPPGKYTDESGLLAYYEVCTFLNEGATEVWDAPQEVPYAYQGNEWVGYDNVRSFKLKAQWLKDNNLGGAVVWPLDMDDFSGSFCHQRHFPLTSTLKGDLNIHSASCKGPY

SEQ ID NO: 16 - protein, 558 aa, Mus musculus
MFVRMLVFRVLLLIALVGKSVIGLSLMSFIAPPDPDWTPDDYYYSYEQSSPDEDPSVTQTTPENPDWYYEDDDPCQSNPCEHGGDCIIRGDTFSCSCPAPFSGSRCQTAQNKCKDNPCVHGDCLITQKHPYYRCACKYPYTGPDCSKVLPACRPNPCQNGGVCSRHRRRSRFTCACPDQYKGKFCEIGPDDCYVGDGYSYRGKVSKTVNQNPCLYWNSHLLLQETYNMFMEDAETHGIAEHNFCRNPDGDHKPWCFVKVNSEKVKWEYCDVTVCPVPDTPNPVESLLEPVMELPGFESCGKTEVAEHAVKRIYGGFKSTAGKHPWQVSLQTSLPLTTSMPQGHFCGGALIHPCWVLTAAHCTDINTKHLKVVLGDQDLKKTESHEQTFRVEKILKYSQYNERDEIPHNDIALLKLKPVGGHCALESRYVKTVCLPSDPFPSGTECHISGWGVTETGEGSRQLLDAKVKLIANPLCNSRQLYDHTIDDSMICAGNLQKPGSDTCQGDSGGPLTCEKDGTYYVYGIVSWGQECGKKPGVYTQVTKFLNWIKTTMHREAGL

SEQ ID NO: 17 - protein, 1312 aa, Mus musculus
MGAASGQRGRWPLSPPLLMLSLLVLLLQPSPAPALDPGLQPGNFSPDEAGAQLFAESYNSSAEVVMFQSTVASWAHDTNITEENARRQEEAALVSQEFAEVWGKKAKELYESIWQNFTDSKLRRIIGSIRTLGPANLPLAQRQQYNSLLSNMSRIYSTGKVCFPNKTATCWSLDPELTNILASSRSYAKLLFAWEGWHDAVGIPLKPLYQDFTAISNEAYRQDDFSDTGAFWRSWYESPSFEESLEHIYHQLEPLYLNLHAYVRRALHRRYGDKYVNLRGPIPAHLLGDMWAQSWENIYDMVVPFPDKPNLDVTSTMVQKGWNATHMFRVSEEFFTSLGLSPMPPEFWAESMLEKPTDGREVVCHASAWDFYNRKDFRIKQCTRVTMEQLATVHHEMGHVQYYLQYKDLHVSLRRGANPGFHEAIGDVLALSVSTPAHLHKIGLLDHVTNDIESDINYLLKMALEKIAFLPFGYLVDQWRWGVFSGRTPPSRYNFDWWYLRTKYQGICPPVARNETHFDAGAKFHIPNVTPYIRYFVSFVLQFQFHQALCKEAGHQGPLHQCDIYQSAQAGAKLKQVLQAGCSRPWQEVLKDLVGSDALDAKALLEYFQPVSQWLEEQNQRNGEVLGWPENQWRPPLPDNYPEGIDLETDEAKADRFVEEYDRTAQVLLNEYAEANWQYNTNITIEGSKILLEKSTEVSNHTLKYGTRAKTFDVSNFQNSSIKRIIKKLQNLDRAVLPPKELEEYNQILLDMETTYSLSNICYTNGTCMPLEPDLTNMMATSRKYEELLWAWKSWRDKVGRAILPFFPKYVEFSNKIAKLNGYTDAGDSWRSLYESDNLEQDLEKLYQELQPLYLNLHAYVRRSLHRHYGSEYINLDGPIPAHLLGNMWAQTWSNIYDLVAPFPSAPNIDATEAMIKQGWTPRRIFKEADNFFTSLGLLPVPPEFWNKSMLEKPTDGREVVCHPSAWDFYNGKDFRIKQCTSVNMEDLVIAHHEMGHIQYFMQYKDLPVTFREGANPGFHEAIGDIMALSVSTPKHLYSLNLLSTEGSGYEYDINFLMKMALDKIAFIPFSYLIDQWRWRVFDGSITKENYNQEWWSLRLKYQGLCPPVPRSQGDFDPGSKFHVPANVPYVRYFVSFIIQFQFHEALCRAAGHTGPLHKCDIYQSKEAGKLLADAMKLGYSKPWPEAMKLITGQPNMSASAMMNYFKPLTEWLVTENRRHGETLGWPEYNWAPNTARAEGSTAESNRVNFLGLYLEPQQARVGQWVLLFLGVALLVATVGLAHRLYNIRNHHSLRRPHRGPQFGSEVELRHS

SEQ ID NO: 18 - protein, 756 aa, Mus musculus
MAKRTFSTLEAFLIFLLVIMTVITVALLTLLFVTSGTIENHKDSGNHWFSTTLGSTTTQPPPITQTPNFPSFRNFSGYYIGVGRADCTGQVSDINLMGYGKNGQNARGLLTRLFSRAFILADPDGSNRMAFVSVELCMISQRLRLEVLKRLESKYGSLYRRDNVILSAIHTHSGPAGFFQYTLYILASEGFSNRTFQYIVSGIMKSIDIAHTNLKPGKIFINKGNVANVQINRSPSSYLLNPQSERARYSSNTDKEMLVLKLVDLNGEDLGLISWFAIHPVSMNNSNHFVNSDNMGYAAYLFEQEKNKGYLPGQGPFVAGFASSNLGDVSPNILGPHCVNTGESCDNDKSTCPNGGPSMCMASGPGQDMFESTHIIGRIIYQKAKELYASASQEVTGPVLAAHQWVNMTDVSVQLNATHTVKTCKPALGYSFAAGTIDGVSGLNITQGTTEGDPFWDTLRDQLLGKPSEEIVECQKPKPILLHSGELTIPHPWQPDIVDVQIVTVGSLAIAAIPGELTTMSGRRFREAIKKEFALYGMKDMTVVIAGLSNVYTHYITTYEEYQAQRYEAASTIYGPHTLSAYIQLFRDLAKAIATDTVANMSSGPEPPFFKNLIASLIPNIADRAPIGKHFGDVLQPAKPEYRVGEVVEVIFVGANPKNSAENQTHQTFLTVEKYEDSVADWQIMYNDASWETRFYWHKGILGLSNATIYWHIPDTAYPGIYRIRYFGHNRKQELLKPAVILAFEGISSPFEVVTT

SEQ ID NO: 19 - protein, 597 aa, Mus musculus
MELSSGVCPATRLQEAEKAAVHKRSPKVLEALRKLNIQADQAPVIAVLGSGGGLRAHIACLGVLSELKELGLLDAVTYLAGVSGSTWALSSLYTKNGNMEGIEEELKHRYEKNEWDFHESLEKAIQASKRENYSLTDFWAYLIVSRQIRELQDSNLSSLKKQVEEGVLPYPIFAAIDEDLLADWRERKTQNSWFEFTPHHAGYPALGAYVPITEFGSRFENGKLVKSEPERDLTFLRGLWGSAFADIKEIKNYILNYFRNPFGKLKFIEGPVTYSEAPRMNVDAMLLDLVMAYFTDMNDPSIKDKLCALQQALGTETDEFGIEMAEIIQNWNETSAEKKEQFLDHLLDRFKKTQEDTTTYSLMNWNTGLVWDRCVFVNETRKCVSKWQWGTVYNFLYKHGKIADETMCSRELLHLVDAGFAINTPYPLVLPPVRETHLILSFDFSAGDPLETIRATADYCQRHEIPFPEVSEDQLKEWAKAPASCYVLRGETGPVVMHFTLFNKDNCGDDIETWRKKYGTVKLSDSYTPDLVRDLLRVSKENVKKNKINILSEMRKVAGNPGNIPRVNKEACLGDRVKDPQGSQTVEFKKSHNISKD

SEQ ID NO: 20 - protein, 117 aa, Mus musculus
MLTFNKMKTTTCSLLICISLLQLMVPVNTEGTLESIVEKKVKELLANRDDCPSTVTKTFSCTSITASGRLASCPSGMTVTGCACGYGCGSWDIRDGNTCHCQCSTMDWATARCCQLA

SEQ ID NO: 21 - protein, 817 aa, Mus musculus
MNSIPVLTLLGVTALLPCVPALTCQKSSAQAVRNVAELPLRWWGAGEKTCEVSEGCQDLIMLLYNGPKVNLVIIKGCTEVEDQEPKVIWLRTGPGLSVVSYTRVCRHGDLCNDVNSTKILEELPTPTVPGSLRCPLCLSNDSCENAPEQVCPVGSTHCYDGVLRLRGDGIRTNLKVQGCMAQPDCNLLNGTQAIGTLYMSENCDLIGPQALDCNSGSLETVRNVSDLHLSWTTGWQTCEAGEGCYETVMLIQNGHEFHMVLTKGCTRDMNKKARLTRHRTGPGISIVSYVHVCRDRDFCNDLSTTDPLWTPPPDTELGTLRCRHCLSTGSCVSASELVCPAGSTHCYSGVLSLRGGGVISDLKVQGCISQSQPGCNLLNGTQTIGPVDVREDCGLQLDALKCQHGTLKTIQDISKLPLQWTAGQKICNVGEGCQDTLMLIENGEQVNLVLTKGCTTAKDQEAKVTEHRTGPGLSVTSYTRVCRKKDFCNDLSTTAPLWAPPPVTAPGTTRCPLCFSEQACENAPEQVCPAGSTHCYSGVLSLRGGGIISDLKVQGCMSQPGCNLLNGTQTIGPVDVSERCSPPSETTELSCYRGVMFELGNGFAEEPVKWTAPGSQVCAPDEICQETLLLIDVGQKSAFLGSKGCSSPGAQDNIGVSIFSRLPGMLVASYTKFCSSHLCNGADSSSVLLSILPRPDVPPPGDVQCPMCVELFGSCKSTDSVTCPRGATHCYKGDIALQGGGLTTRVSIQGCMAPPIKPLLGDSKTIGIFSAEESSNYRHEDDVTSAPSLAWTLRLSAWMLGLSALLSSLYAGICPLC

SEQ ID NO: 22 - protein, 219 aa, Mus musculus
MEKLIVGILFLSVLSGSVAQTDMKGKAFIFPQESSTAYVSLIPKVRKSLQNFTLCMKAFTDLTRPYSIFSYNTRTKDNEILLFVENIGEYMFYVGNLVAIFKAPTNLPDPVRICVNWESVSGIAEFWLNGKPLGRKGLNKGYTVGGDAMIIIGQEQDSFGGNFDAKQSFVGEIWDVSLWDHVVPLEKVSDSCNNGNLINWQALNYEDNGYVVIKPKLWP

SEQ ID NO: 23 - protein, 397 aa, Mus musculus
MKPLLVLLLLLLLDLAQAQGALHRVPLRRHQSLRKKLRAQGQLSEFWRSHNLDMTRLSESCNVYSSVNEPLINYLDMEYFGTISIGTPPQNFTVIFDTGSSNLWVPSVYCTSPACKAHPVFHPSQSDTYTEVGNHFSIQYGTGSLTGIIGADQVSVEGLTVDGQQFGESVKEPGQTFVNAEFDGILGLGYPSLAAGGVTPVFDNMMAQNLVALPMFSVYLSSDPQGGSGSELTFGGYDPSHFSGSLNWIPVTKQAYWQIALDGIQVGDTVMFCSEGCQAIVDTGTSLITGPPDKIKQLQEAIGATPIDGEYAVDCATLDTMPNVTFLINEVSYTLNPTDYILPDLVEGMQFCGSGFQGLDIPPPAGPLWILGDVFIRQFYSVFDRGNNQVGLAPAVP

SEQ ID NO: 24 - protein, 730 aa, Mus musculus
MSPWQPLLLALLAFGCSSAAPYQRQPTFVVFPKDLKTSNLTDTQLAEAYLYRYGYTRAAQMMGEKQSLRPALLMLQKQLSLPQTGELDSQTLKAIRTPRCGVPDVGRFQTFKGLKWDHHNITYWIQNYSEDLPRDMIDDAFARAFAVWGEVAPLTFTRVYGPEADIVIQFGVAEHGDGYPFDGKDGLLAHAFPPGAGVQGDAHFDDDELWSLGKGVVIPTYYGNSNGAPCHFPFTFEGRSYSACTTDGRNDGTPWCSTTADYDKDGKFGFCPSERLYTEHGNGEGKPCVFPFIFEGRSYSACTTKGRSDGYRWCATTANYDQDKLYGFCPTRVDATVVGGNSAGELCVFPFVFLGKQYSSCTSDGRRDGRLWCATTSNFDTDKKWGFCPDQGYSLFLVAAHEFGHALGLDHSSVPEALMYPLYSYLEGFPLNKDDIDGIQYLYGRGSKPDPRPPATTTTEPQPTAPPTMCPTIPPTAYPTVGPTVGPTGAPSPGPTSSPSPGPTGAPSPGPTAPPTAGSSEASTESLSPADNPCNVDVFDAIAEIQGALHFFKDGWYWKFLNHRGSPLQGPFLTARTWPALPATLDSAFEDPQTKRVFFFSGRQMWVYTGKTVLGPRSLDKLGLGPEVTHVSGLLPRRLGKALLFSKGRVWRFDLKSQKVDPQSVIRVDKEFSGVPWNSHDIFQYQDKAYFCHGKFFWRVSFQNEVNKVDHEVNQVDDVGYVTYDLLQCP

SEQ ID NO: 25 - protein, 374 aa, Mus musculus
MGLLVFVRNLLLALCLFLVLGFLYYSAWKLHLLQWEDSNSLLLSLDSAGQTLGTEYDRLGFLLKLDSKLPAELATKYANFSEGACKPGYASAMMTAIFPRFSKPAPMFLDDSFRKWARIREFVPPFGIKGQDNLIKAILSVTKEYRLTPALDSLHCRRCIIVGNGGVLANKSLGSRIDDYDIVIRLNSAPVKGFERDVGSKTTLRITYPEGAMQRPEQYERDSLFVLAGFKWQDFKWLKYIVYKERVSASDGFWKSVATRVPKEPPEIRILNPYFIQEAAFTLIGLPFNNGLMGRGNIPTLGSVAVTMALHGCDEVAVAGFGYDMNTPNAPLHYYETVRMAAIKESWTHNIQREKEFLRKLVKARVITDLSSGI

SEQ ID NO: 26 - protein, 307 aa, Mus musculus
MVPVLLSLPLLLGPAVFQETGSYYLTFLYTGLSRPSKGFPRFQATAFLNDQAFFHYNSNSGKAEPVGPWSQVEGMEDWEKESQLQRAREEIFLVTLKDIMDYYKDTTGSHTFQGMFGCEITNNRSSGAVWRYAYDGEDFIEFNKEIPAWIPLDPAAANTKLKWEAEKVYVQRAKAYLEEECPEMLKRYLNYSRSHLDRIDPPTVTITSRVIPGGNRIFKCLAYGFYPQRISLHWNKANKKLAFEPERGVFPNGNGTYLSWAEVEVSPQDIDPFFCLIDHRGFSQSLSVQWDRTRKVKDENNVVAQPQ

SEQ ID NO: 27 - protein, 247 aa, Mus musculus
MLLLQALLFLLILPSHAEDDVTTTEELAPALVPPPKGTCAGWMAGIPGHPGHNGTPGRDGRDGTPGEKGEKGDAGLLGPKGETGDVGMTGAEGPRGFPGTPGRKGEPGEAAYVYRSAFSVGLETRVTVPNVPIRFTKIFYNQQNHYDGSTGKFYCNIPGLYYFSYHITVYMKDVKVSLFKKDKAVLFTYDQYQEKNVDQASGSVLLHLEVGDQVWLQVYGDGDHNGLYADNVNDSTFTGFLLYHDTN

SEQ ID NO: 28 - protein, 223 aa, Homo sapiens
MNKPLLWISVLTSLLEAFAHTDLSGKVFVFPRESVTDHVNLITPLEKPLQNFTLCFRAYSDLSRAYSLFSYNTQGRDNELLVYKERVGEYSLYIGRHKVTSKVIEKFPAPVHICVSWESSSGIAEFWINGTPLVKKGLRQGYFVEAQPKIVLGQEQDSYGGKFDRSQSFVGEIGDLYMWDSVLPPENILSAYQGTPLPANILDWQALNYEIRGYVIIKPLVWV

SEQ ID NO: 29 - protein, 251 aa, Homo sapiens
MTRLTVLALLAGLLASSRAGSSPLLDIVGGRKARPRQFPFLASIQNQGRHFCGGALIHARFVMTAASCFQSQNPGVSTVVLGAYDLRRRERQSRQTFSISSMSENGYDPQQNLNDLMLLQLDREANLTSSVTILPLPLQNATVEAGTRCQVAGWGSQRSGGRLSRFPRFVNVTVTPEDQCRPNNVCTGVLTRRGGICNGDGGTPLVCEGLAHGVASFSLGPCGRGPDFFTRVALFRDWIDGVLNNPGPGPA

SEQ ID NO: 30 - protein, 1065 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 31 - protein, 255 aa, Homo sapiens
MQPLLLLLAFLLPTGAEAGEIIGGRESRPHSRPYMAYLQIQSPAGQSRCGGFLVREDFVLTAAHCWGSNINVTLGAHNIQRRENTQQHITARRAIRHPQYNQRTIQNDIMLLQLSRRVRRNRNVNPVALPRAQEGLRPGTLCTVAGWGRVSMRRGTDTLREVQLRVQRDRQCLRIFGSYDPRRQICVGDRRERKAAFKGDSGGPLLCNNVAHGIVSYGKSSGVPPEVFTRVSSFLPWIRTTMRSFKLLDQMETPL

SEQ ID NO: 32 - protein, 267 aa, Homo sapiens
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH

SEQ ID NO: 33 - protein, 710 aa, Homo sapiens
MKLVFLVLLFLGALGLCLAGRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 34 - protein, 745 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 35 - protein, 201 aa, Homo sapiens
MALSWVLTVLSLLPLLEAQIPLCANLVPVPITNATLDQITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGES

SEQ ID NO: 36 - protein, 196 aa, Homo sapiens
MSRRSMLLAWALPSLLRLGAAQETEDPACCSPIVPRNEWKALASECAQHLSLPLRYVVVSHTAGSSCNTPASCQQQARNVQHYHMKTLGWCDVGYNFLIGEDGLVYEGRGWNFTGAHSGHLWNPMSIGISFMGNYMDRVPTPQAIRAAQGLLACGVAQGALRSNYVLKGHRDVQRTLSPGNQLYHLIQNWPHYRSP

SEQ ID NO: 37 - protein, 148 aa, Homo sapiens
MKLLVLAVLLTVAAADSGISPRAVWQFRKMIKCVIPGSDPFLEYNNYGCYCGLGGSGTPVDELDKCCQTHDNCYDQAKKLDSCKFLLDNPYTHTYSYSCSGSAITCSSKNKECEAFICNCDRNAAICFSKAPYNKAHKNLDTKKYCQS

SEQ ID NO: 38 - protein, 247 aa, Homo sapiens
MNLLLILTFVAAAVAAPFDDDDKIVGGYICEENSVPYQVSLNSGYHFCGGSLISEQWVVSAGHCYKSRIQVRLGEHNIEVLEGNEQFINAAKIIRHPKYNSRTLDNDILLIKLSSPAVINSRVSAISLPTAPPAAGTESLISGWGNTLSSGADYPDELQCLDAPVLSQAECEASYPGKITNNMFCVGFLEGGKDSCQGDSGGPVVSNGELQGIVSWGYGCAQKNRPGVYTKVYNYVDWIKDTIAANS

SEQ ID NO: 39 - protein, 256 aa, Homo sapiens
MAHRPPSPALASVLLALLLSGAARAAEIVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRRVEAKGRP

SEQ ID NO: 40 - protein, 161 aa, Homo sapiens
MVPKLFTSQICLLLLLGLLAVEGSLHVKPPQFTWAQWFETQHINMTSQQCTNAMQVINNYQRRCKNQNTFLLTTFANVVNVCGNPNMTCPSNKTRKNCHHSGSQVPLIHCNLTTPSPQNISNCRYAQTPANMFYIVACDNRDQRRDPPQYPVVPVHLDRII

SEQ ID NO: 41 - protein, 92 aa, Homo sapiens
MTKLEEHLEGIVNIFHQYSVRKGHFDTLSKGELKQLLTKELANTIKNIKDKAVIDEIFQGLDANQDEQVDFQEFISLVAIALKAAHYHTHKE

SEQ ID NO: 42 - protein, 93 aa, Homo sapiens
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 43 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 44 - protein, 222 aa, Homo sapiens
MLSRAVCGTSRQLAPVLGYLGSRQKHSLPDLPYDYGALEPHINAQIMQLHHSKHHAAYVNNLNVTEEKYQEALAKGDVTAQIALQPALKFNGGGHINHSIFWTNLSPNGGGEPKGELLEAIKRDFGSFDKFKEKLTAASVGVQGSGWGWLGFNKERGHLQIAACPNQDPLQGTTGLIPLLGIDVWEHAYYLQYKNVRPDYLKAIWNVINWENVTERYMACKK

SEQ ID NO: 45 - protein, 309 aa, Homo sapiens
MLLILLSVALLALSSAQSLNEDVSQEESPSVISGKPEGRRPQGGNQPQRTPPPPGKPEGRPPQGGNQSQGPPPRPGKPEGPPPQGGNQSQGPPPRPGKPEGQPPQGGNQSQGPPPRPGKPEGPPPQGGNQSQGPPPRPGKPEGPPPQGGNQSQGPPPHPGKPEGPPPQGGNQSQGPPPRPGKPEGPPPQGGNQSQGPPPRPGKPEGPPPQGGNQSQGPPPRPGKPEGSPSQGGNKPQGPPPHPGKPQGPPPQEGNKPQRPPPPGRPQGPPPPGGNPQQPLPPPAGKPQGPPPPPQGGRPHRPPQGQPPQ

SEQ ID NO: 46 - protein, 388 aa, Homo sapiens
MKWMVVVLVCLQLLEAAVVKVPLKKFKSIRETMKEKGLLGEFLRTHKYDPAWKYRFGDLSVTYEPMAYMDAAYFGEISIGTPPQNFLVLFDTGSSNLWVPSVYCQSQACTSHSRFNPSESSTYSTNGQTFSLQYGSGSLTGFFGYDTLTVQSIQVPNQEFGLSENEPGTNFVYAQFDGIMGLAYPALSVDEATTAMQGMVQEGALTSPVFSVYLSNQQGSSGGAVVFGGVDSSLYTGQIYWAPVTQELYWQIGIEEFLIGGQASGWCSEGCQAIVDTGTSLLTVPQQYMSALLQATGAQEDEYGQFLVNCNSIQNLPSLTFIINGVEFPLPPSSYILSNNGYCTVGVEPTYLSSQNGQPLWILGDVFLRSYYSVYDLGNNRVGFATAA

SEQ ID NO: 47 - protein, 323 aa, Homo sapiens
MAYVPAPGYQPTYNPTLPYYQPIPGGLNVGMSVYIQGVASEHMKRFFVNFVVGQDPGSDVAFHFNPRFDGWDKVVFNTLQGGKWGSEERKRSMPFKKGAAFELVFIVLAEHYKVVVNGNPFYEYGHRLPLQMVTHLQVDGDLQLQSINFIGGQPLRPQGPPMMPPYPGPGHCHQQLNSLPTMEGPPTFNPPVPYFGRLQGGLTARRTIIIKGYVPPTGKSFAINFKVGSSGDIALHINPRMGNGTVVRNSLLNGSWGSEEKKITHNPFGPGQFFDLSIRCGLDRFKVYANGQHLFDFAHRLSAFQRVDTLEIQGDVTLSYVQI